(19) United States
(12) Patent Application Publication
(10) Pub. No.: US 2026/0200994 A1
(43) Pub. Date: Jul. 16, 2026

(54) EXPRESSION OF ANTIGEN-ADJUVANT FUSION SUBUNIT PROTEINS IN DIATOMS, AND METHODS OF GENERATION AND USE THEREOF

(71) Applicant: PhycoVax LLC, Carlsbad, CA (US)

(72) Inventors: Roshan SHRESTHA, Poway, CA (US); Thomas GRIMM, Carlsbad, CA (US)

(21) Appl. No.: 19/127,739

(22) PCT Filed: Oct. 20, 2023
(86) PCT No.: PCT/US2023/077475

§ 371 Date: May 7, 2025

Related U.S. Application Data
(60) Provisional application No. 63/424,426, filed on Nov. 10, 2022.
(60) Provisional application No. 63/437,921, filed on Jan. 9, 2023.

(51) Int. Cl. C07K 14/255; A61K 9/00; A61K 36/02; A61K 38/00; C12N 15/79

(52) U.S. Cl. CPC C07K 14/255; A61K 9/0053; A61K 36/02; A61K 38/00; C12N 15/79; C07K 2319/00; C12N 2800/10

(57) ABSTRACT

This application relates to compositions and methods used to treat animals with at least one microbial protein, such as a flagellin protein. In embodiments, application relates to compositions and methods to treat animals with a fusion protein comprising at least one adjuvant protein, such as a flagellin protein. In embodiments, the composition further includes at least one adjuvantic protein fused with at least one antigen protein, and the fusion protein is expressed in diatoms.

### CROSS-REFERENCE TO RELATED APPLICATIONS

This application claims the benefit of U.S. Provisional Patent Application No. 63/424,426, filed Nov. 10, 2022 and entitled “Treatment of Shrimp with Proteins from Microalgae,” and U.S. Provisional Patent Application No. 63/437,921 filed Jan. 9, 2023 and entitled “Expression of Antigen-Adjuvant Fusion Subunit Proteins in Diatoms, and Methods of Generation and Use Thereof,” the disclosures of each of which are hereby incorporated by reference in their entirety.

### FIELD

This application relates to the expression of molecular adjuvant-antigen fusion subunit proteins in diatoms.

### SEQUENCE LISTING

The instant application contains a Sequence Listing which has been submitted electronically in xml format and is hereby incorporated by reference in its entirety. Said xml copy was created on Oct. 12, 2023, is named 89566.00116.xml, and is 85.8 kilobytes in size.

### BACKGROUND

The growing global popularity and demand for Whiteleg shrimp (Litopenaeus vannamei) necessitates that the shrimp aquaculture industry ensures that there is a reliable production of shrimp, in both quantity and quality. The cultivation process is contingent upon the survival of the crustaceans, especially in their early development stages. However, during the development stages, crustaceans have very high mortality rates due to Vibrio and other pathogens. Thus, treatments are needed to increase the survival rates of the Whiteleg shrimp.

Oral vaccine delivery is simple, cost-effective, induces minimal stress and side effects, and is suitable for mass immunization of animals, including shrimp, fish, poultry birds, swine and cattle of all sizes. Oral vaccines aim to stimulate strong mucosal immune responses at the mucosal sites and protect the animal from infection when corresponding pathogens are encountered. However, a lack of mucosal adjuvants hinders the development of effective mucosal vaccines.

### SUMMARY

In an aspect, a composition is provided comprising a sequence having at least 80% sequence identity with any of SEQ ID NOs: 3, 12, 13, 14, 15, 16, 17, 18, and 19.

In embodiments, the sequence has at least 85%, at least 90%, or at least 95% sequence identity with any of SEQ ID NOs: 3, 12, 13, 14, 15, 16, 17, 18, and 19. In embodiments, the sequence comprises any of SEQ ID NOs: 3, 12, 13, 14, 15, 16, 17, 18, and 19. In embodiments, the sequence is part of a fusion protein.

In an aspect, a composition is provided, comprising a sequence having at least 80% sequence identity with SEQ ID NO: 4.

In embodiments, the sequence has at least 85%, at least 90%, or at least 95% identity with SEQ ID NO: 4. In embodiments, the sequence comprises SEQ ID NO: 4. In embodiments, the sequence is part of a fusion protein. In embodiments, the fusion protein comprises an adjuvantic protein.

In an aspect, a method of treating an animal is provided, comprising administering a therapeutically effective amount of at least one protein derived from a microbe to the animal.

In embodiments, the at least one protein derived from a microbe comprises flagellin. In embodiments, the flagellin comprises a sequence having at least 80% sequence identity with SEQ ID NO: 4. In embodiments, the flagellin comprises a sequence having at least 85%, at least 90%, at least 95% with SEQ ID NO: 4. In embodiments, the flagellin comprises SEQ ID NO: 4. In embodiments, the flagellin comprises a codon-optimized flagellin. In embodiments, the codon-optimized flagellin comprises at least 80% sequence identity with any one or more of SEQ ID NO: 3, 12, 13, 14, 15, 16, 17, 18, and 19. In embodiments, the codon-optimized flagellin comprises at least 85%, at least 90%, at least 95% sequence identity with any of one or more of SEQ ID NO: 3, 12, 13, 14, 15, 16, 17, 18, and 19. In embodiments, the codon-optimized flagellin comprises any one or more of SEQ ID NO: 3, 12, 13, 14, 15, 16, 17, 18, and 19.

In embodiments, the flagellin is incorporated into at least one diatom. In embodiments, the at least one diatom comprises any one or more of Thalassiosira pseudonana, Thalassiosira weissflogii, Cyclotella cryptica, or Chaetoceros muelleri.

In embodiments, the animal comprises shrimp, fish, poultry, swine, cattle, sheep, or goats. In embodiments, the animal comprises shrimp. In embodiments, the animal comprises fish. In embodiments, the fish comprises Tilapia fish.

In embodiments, administering the therapeutically effective amount of the at least one protein comprises orally administering the at least one protein. In embodiments, orally administering the effective amount of the at least one protein comprises using a tablet or capsule to orally administer the protein. In embodiments, administering the effective amount of the at least one protein comprises injecting the at least one protein into the animal. In embodiments, injecting the at least one protein comprises intravenously injecting the at least one protein, intradermally injecting the at least one protein, subcutaneously injecting the at least one protein, or intramuscularly injecting the at least one protein.

In embodiments, the at least one protein comprises a fusion protein. In embodiments, the fusion protein comprises any one or more of (i) an antigen, (ii) a hormone, (iii) a toxin, and (iv) an antibody. In embodiments, the treatment of the animal is a prophylactic treatment. In embodiments, the treatment of the animal is a therapeutic treatment.

In an aspect, a method of treating an animal is provided, comprising: adding at least one protein from a microbe to a diatom cell; and administering the diatom cell to the animal.

In embodiments, the method further comprises incorporating the diatom cell into animal feed, and administering the diatom cell and the animal feed to the animal. In embodiments, the animal comprises shrimp. In embodiments, the animal comprises fish. In embodiments, the fish comprises Tilapia fish.

In embodiments, the at least one protein comprises flagellin. In embodiments, the flagellin comprises a sequence having at least 80% sequence identity with SEQ ID NO: 4. In embodiments, the sequence has at least 85%, at least 90%, at least 95% identity with SEQ ID NO: 4. In embodiments, the flagellin comprises a codon-optimized flagellin, and wherein the codon-optimized flagellin comprises a sequence that has least 80% sequence identity with any one or more of SEQ ID NOs: 3, 12, 13, 14, 15, 16, 17, 18, and 19. In embodiments, the sequence has at least 85%, at least 90%, at least 95% identity with any one or more of SEQ ID NOs: 3, 12, 13, 14, 15, 16, 17, 18, and 19.

In embodiments, the diatom comprises Thalassiosira pseudonana, Thalassiosira weissflogii, Cyclotella cryptica, or Chaetoceros muelleri.

In embodiments, adding the at least one protein from a microbe to the diatom results in expression of the at least one protein in any one or more of the cytoplasm, the plasma membrane, the chloroplast, the endoplasmic reticulum, or the chloroplast endoplasmic reticulum, of the diatom.

In embodiments, the diatoms are incorporated into the animal feed through use of any one or more of infusion, spray coating, and top coating. In embodiments, the diatoms are incorporated into the animal feed with the use of a binding agent. In embodiments, the diatoms are incorporated into the animal feed without the use of a binding agent.

In an aspect, a method of feeding an animal is provided, comprising administering a diatom to the animal, wherein the diatom expresses at least one flagellin protein.

In embodiments, the at least one flagellin protein comprises at least 80% sequence identity with SEQ ID NO: 4. In embodiments, the at least one flagellin protein comprises at least 85%, at least 90%, or at least 95% sequence identity with SEQ ID NO: 4. In embodiments, the at least one flagellin protein comprises at least one codon optimized flagellin protein. In embodiments, the at least one codon optimized flagellin protein comprises a sequence that has at least 80% sequence identity with any one or more of SEQ ID NOs: 3, 12, 13, 14, 15, 16, 17, 18, and 19. In embodiments, the at least one codon optimized flagellin protein comprises a sequence that has at least 85%, at least 90%, or at least 95% sequence identity with any one or more of SEQ ID NOs: 3, 12, 13, 14, 15, 16, 17, 18, and 19.

In embodiments, the diatom comprises Thalassiosira pseudonana, Thalassiosira weissflogii, Cyclotella cryptica, or Chaetoceros muelleri.

In embodiments, the flagellin derives from a microbe. In embodiments, the microbe comprises Salmonella enterica, Bacillus subtilis, Vibrio anguillarum, Pseudomonas aeruginosa, Salmonella typhimurium, Serratia marcescens, Edwardsiella tarda, Proteus mirabilis, Escherichia coli, Shigella flexneri, Listeria monocytogenes, Bartonella bacilliformis, Rhizobium meliloti, Campylobacter jejuni, Campylobacter coli, Helicobacter pylori, Helicobacter felis, Helicobacter hepaticus, Legionella pneumophila, Salmonella marcescens, or Wolinella succinogenes.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF THE DRAWINGS

FIGS. 1A and 1B schematically depict vector maps showing embodiments of fusion proteins as described herein.

FIGS. 2A-2C provide results showing the expression of flagellin-Vibrio parahaemolyticus (Vp) Outer membrane protein (OMP) fusion protein in centric diatoms. FIG. 2A shows expression of flagellin-Vp OMP fusion protein in Thalassiosira pseudonana. FIG. 2B shows expression of flagellin-Vp OMP fusion protein in Thalassiosira weissflogii (Conticribra weissflogii). FIG. 2C shows expression of flagellin-VP OMP fusion protein in the centric diatom Cyclotella cryptica. In each of FIGS. 2A-2C the bright white shows the C-terminus GFP tag.

FIG. 3 provides results showing a time-course expression analysis of eGFP-tagged Vp OMP fusion protein with and without flagellin in Thalassiosira pseudonana.

FIGS. 4A-4B provide results of challenge trial experiments with Vibrio parahaemolyticus. FIG. 4A provides a Kaplan-Meier representation of cumulative survival of shrimp Penaeus vannamei fed with control (wild type Thalassiosira pseudonana) and test diets (flagellin-Vp OMP fusion protein expressed in Thalassiosira pseudonana) that were challenged on the 7th day with Vibrio parahaemolyticus by immersion. Mortality was recorded for the next 7 days. FIG. 4B provides a Kaplan-Meier representation of cumulative survival of shrimp Penaeus vannamei fed with control (wild-type Cylotella cryptica) and test diets (flagellin-Vp OMP fusion protein expressed in Cylotella cryptica) that were challenged on the 7th day with Vibrio parahaemolyticus by immersion. Mortality was recorded for the next 7 days.

FIGS. 5A-5B provide results of superoxide dismutase (SOD) activity assays in hemolymph of shrimp Penaeus vannamei fed diets with control and test diets for 60 days. In FIG. 5A, the control diet was commercial shrimp feed, the test diet 0 was wild-type Thalassiosira pseudonana and the test diet 1 was flagellin-Vp OMP fusion protein expressed in Thalassiosira pseudonana. In FIG. 5B, the control diet was wild-type Cyclotella cryptica, and the test diet was flagellin-Vp OMP fusion protein expressed in Cyclotella cryptica.

FIGS. 6A-6B depict data showing the average survival rate of shrimp after receiving various treatments (i.e., T1, T2, and T3) relative to the survival rate of the shrimp treated with controls.

FIG. 7 depicts a pathology analysis of the hepatopancreas of the shrimp after receiving various treatments (i.e., T1, T2, and T3) relative to the shrimp treated with controls.

FIGS. 8A-8D depict data showing various grades of clinical signs, such as lesions, in the hepatopancreas of the shrimp after receiving various treatments (i.e., T1, T2, and T3).

FIG. 9 depicts data showing the presence of lipid cells in the tubules of the hepatopancreas of the shrimp after receiving various treatments (i.e., T1, T2, and T3).

FIG. 10 depicts data showing the food conversion factor of the shrimp after receiving various treatments (i.e., T1, T2, and T3) versus the control-treated shrimp.

FIG. 11 depicts data showing vibrio disease prevalence analysis of shrimp after various treatments (i.e., T1, T2, and T3) relative to control-treated shrimp.

FIG. 12 depicts a table showing the parameters for each cage enclosure for the shrimp under the various treatment schemes.

FIG. 13 depicts data showing the results of immunization regimens for Tilapia fish.

### DETAILED DESCRIPTION

Embodiments provided herein relate to compositions that contain one or more proteins derived from bacteria, such as a flagellin protein. Embodiments provided herein also relate to methods in which one or more proteins from microalgae (e.g., diatoms) are used to treat animals such as shrimp and fish. Embodiments provided herein also relate to methods in which one or more proteins from microalgae are used to feed animals such as shrimp and fish. The one or more proteins from microalgae (e.g., diatoms) may be derived from a microbe and may be exogenously expressed in the microalgae.

In some embodiments, one or more proteins from microalgae function as any one or more of vaccines, immunomodulators, immunostimulants, growth enhancers, and functional feeds. The present protein(s) from microalgae (e.g., diatoms) thus may be considered to “treat” an animal when administered (e.g., fed) to the animal in a “therapeutically effective amount,” that is, an amount which is effective to act as a vaccine, immunomodulator, immunostimulant, growth enhancer, and/or functional feed.

In some embodiments, one or more proteins comprise one or more fusion proteins. In some embodiments, the fusion protein(s) function as any one or more of vaccines, immunomodulators, immunostimulants, growth enhancers, and functional feeds. In some embodiments, any one or more of the subunits within the fusion protein(s) function as any one or more of vaccines, immunomodulators, immunostimulants, and growth enhancers.

In some embodiments, the fusion protein comprises one or more adjuvants. In some embodiments, the adjuvant comprises any one or more of bacterial flagellins, lipoproteins, heat shock proteins, and fibronectins. In some embodiments, the adjuvant comprises one or more domains of fibronectin. In some embodiments, the adjuvant comprises extra domain A of fibronectin.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

As used in the description and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

As used herein, “expression”, “expressed”, or “encodes” refers to the process by which polynucleotides are transcribed into mRNA and/or the process by which the transcribed mRNA is subsequently being translated into peptides, polypeptides, or proteins. Expression may include splicing of the mRNA in a diatom cell or other forms of post-transcriptional modification or post-translational modification.

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

As used herein, the term “comprising” is intended to mean that the compositions and methods include the recited elements, but not excluding others.

The terms “administration of” or “administering” an active agent should be understood to mean providing an active agent to the animal in need of treatment in a form that can be introduced into that animal's body in a therapeutically effective amount.

An animal “in need of treatment” can be any animal (e.g., shrimp or fish) that is in need of an improvement to its immune system, so as to increase the animal's chance of survival.

As used herein, the term “diatom” refers to algae or microalgae belonging to the Phylum Bacillariophyta that contain cell walls (frustules) made up of two overlapping half-shells, the epitheca and hypotheca. Frustules are composed of nanopatterned biogenic or opaline silica. Nonlimiting examples of diatoms include classes Bacillariophyceae (Raphid, pennate diatoms) and Coscinodiscophyceae (centric diatoms). Nonlimiting examples of genera of centric diatoms include Thalassiosira, Cyclotella, Conticribra, and Chaetoceros. Nonlimiting examples of centric diatoms of the genus Thalassiosira include Thalassiosira pseudonana, Thalassiosira weissflogii, Nonlimiting examples of centric diatoms of the genus Conticribra include Conticribra weissflogii. Nonlimiting examples of centric diatoms of the genus Cyclotella include Cyclotella cryptica. Nonlimiting examples of centric diatoms of the genus Chaetoceros include Chaetoceros muelleri. Nonlimiting examples of pennate diatoms of the genus Cylindrotheca include Cylindrotheca fusiformis. Nonlimiting examples of pennate diatoms of the genus Phaeodactylum include Phaeodactylum tricornutum.

As used herein, the phrase “fusion protein” refers to a protein that contains amino acid sequences that are derived from more than one source, such as, for example, more than one gene.

As used herein, reference to a protein being “from” an organism, such as microalgae, is intended to encompass either a protein that is endogenously expressed in the organism or a protein that is exogenously expressed in the organism. A protein that is exogenously expressed in the organism can derive from the same organism in which the protein is expressed or it can derive from a different organism in which the protein is expressed.

As used herein, the phrase “adjuvantic protein” refers to any protein that is used to enhance the immune response of an antigen. In some embodiments, the fusion protein comprises flagellin.

As used herein, the term “microbe” refers to any microorganism, for example, a bacterium such as Salmonella enterica, Bacillus subtilis, Vibrio anguillarum, Pseudomonas aeruginosa, Salmonella typhimurium, Serratia marcescens, Edwardsiella tarda, Proteus mirabilis, Escherichia coli, Shigella flexneri, Listeria monocytogenes, Bartonella bacilliformis, Rhizobium meliloti, Campylobacter jejuni, Campylobacter coli, Helicobacter pylori, Helicobacter felis, Helicobacter hepaticus, Legionella pneumophila, Salmonella marcescens, or Wolinella succinogenes.

As used herein, the phrase “sequence identity” refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the sequence identity for the test sequence(s) relative to the reference sequence, based on the designated program paramenters.

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

The nucleic acid and protein sequences of the present disclosure can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules provided in the disclosure. BLAST protein searches can be performed with the XBLAST program, score=50, word length=3 to obtain amino acid sequences homologous to the protein molecules of the disclosure. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used. See http://www.ncbi.nlm.nih.gov.

As used herein, the term “variant” may also be referred to herein as analog or variation. A variant refers to any substitution, deletion, or addition to a nucleotide sequence or an amino acid sequence.

As used herein, the phrase “therapeutically effective amount” refers to a sufficient quantity of the active agents of the present disclosure, in a suitable composition, and in a suitable dosage form to treat or prevent the symptoms, or onset of complications from a given ailment, disease or condition. An example of ailments, disease, or conditions includes an insufficient immune response by an organism to external/environmental stimuli that can result in death of the organism.

As used herein, the phrase “pharmaceutically acceptable” refers to those compounds, materials, compositions, and/or dosage forms which are, within the scope of sound medical judgment, suitable for use in contact with the tissues, organs, and/or bodily fluids of animals without excessive toxicity, irritation, allergic response, or other problems or complications commensurate with a reasonable benefit/risk ratio.

As used herein, the phrase “shrimp animal feed” refers to food that shrimp eat in their natural habitat. This food can include other small crustaceans as well as benthic organisms, amongst others. The food can be ground or mixed together. The food can be processed and include additives.

As used herein, the phrase “shrimp/fish animal feed” refers to food that fish eat in their natural habitat. The food can include any one or more of algae, algae-derived products, fishmeal, vegetable proteins, and wheat. The food can be ground or mixed together to form pellets of various sizes. The food can be processed and include additives other than the food from the natural habitat.

As used herein, the phrase “flagellin” or “flagellin protein” refers to a structural protein of the flagellum, a surface filament that is involved in bacterial motility. Flagellins have immunostimulatory properties as protein-based vaccine adjuvants. Other instances of such adjuvants that can be used in combination with flagellin include lipoproteins, heat shock proteins like DnaJ (HSP 40), and the extra domain A of fibronectin. Flagellins activate a variety of immune and non-immune cell types, including T cells, B cells, dendritic cells, natural killer (NK) cells, and non-lymphoid cells, primarily through the activation of Toll-like receptor 5 (TLR5). The following references described the structure and function of flagellin and are incorporated by reference herein in their entirities: (i) Wangkahart E, Secombes C J, Wang T: Studies on the use of flagellin as an immunostimulant and vaccine adjuvant in fish aquaculture. Frontiers in Immunology 2018, 9:3054; (ii) Kim M I, Lee C, Park J, Jeon B-Y, Hong M: Crystal structure of Bacillus cereus flagellin and structure-guided fusion-protein designs. Scientific Reports 2018, 8:5814; (iii) Hajam I A, Dar P A, Shahnawaz I, Jaume J C, Lee J H: Bacterial flagellin—a potent immunomodulatory agent. Experimental & Molecular Medicine 2017, 49:e373; (iv) Girard A, Saron W, Bergeron-Sandoval L-P, Sarhan F, Archambault D: Flagellin produced in plants is a potent adjuvant for oral immunization. Vaccine 2011, 29:6695-6703; (v) Pulendran B, Arunachalam P S, O'Hagan D T: Emerging concepts in the science of vaccine adjuvants. Nature Reviews Drug Discovery 2021, 20:454-475; (vi) Doan T-D, Wang H-Y, Ke G-M, Cheng L-T: N-terminus of Flagellin Fused to an Antigen Improves Vaccine Efficacy against Pasteurella multocida Infection in Chickens. Vaccines 2020, 8:283; (vii) Huleatt J. et al., 2007. Vaccination with recombinant fusion proteins incorporating Toll-like receptor ligands induces rapid cellular and humoral immunity. Vaccine 25(4): 763-75; (viii) Mbow M L. et al., 2010. New adjuvants for human vaccines. Curr Opin Immunol. 22 (3):411-6; (ix) Skountzou I. et al., 2010. Salmonella flagellins are potent adjuvants for intranasally administered whole inactivated influenza vaccine. Vaccine 28(4):4103-12; and (x) Miao E A. & Warren S E., 2010. Innate immune detection of bacterial virulence factors via the NLRC4 inflammasome. J Clin Immunol. 30 (4): 502-6.

As used herein, when referring to a protein being “derived from” a vector, it is meant that the protein is obtained by expressing the vector in a cell.

### Compositions

In an aspect, a composition is provided comprising a flagellin protein. In some embodiments, the flagellin protein comprises a synthetic flagellin protein or a naturally occurring flagellin protein. In some embodiments, the flagellin protein comprises a codon-optimized flagellin protein. In some embodiments, the flagellin protein comprises any codon-optimized flagellin protein described herein.

In an aspect, the composition can be part of a fusion protein. In some embodiments, the fusion protein comprises an adjuvantic protein comprising flagellin fused with a heterologous protein.

In some embodiments, the heterologous protein comprises any one or more of antigens, hormones, toxins, antibodies, immunostimulants, growth stimulants, and nanobodies. In some embodiments, the antigen comprises an endogenous antigen, relative to the species from which the flagellin derived. In some embodiments, the antigen comprises an exogenous antigen, relative to the species from which the flagellin derived. In some examples, the antigen comprises an autoantigen, relative to the species from which the flagellin derived. In some examples, the antigen is a tumor antigen. In some embodiments, the toxin comprises a hemotoxin.

In some embodiments, the toxin comprises a hepatotoxin. In some embodiments, the toxin comprises a neurotoxin. In some embodiments, the toxin comprises a phototoxin. In some embodiments, the antibody comprises any of the known isotypes of antibodies (i.e., IgM, IgA, IgD, IgG, and IgE).

In some embodiments, the hormone can be any peptide-derived hormone, any lipid-derived hormone, or any amino acid-derived hormone.

In some embodiments, any of the heterologous proteins described herein derive from any one or more of bacteria, viruses, and parasites.

In some embodiments, any composition described herein can be added to a diatom. In some embodiments, the composition can be added to any one or more of four centric diatoms. In some embodiments, the genus of centric diatoms comprises Thalassiosira. In some embodiments, the centric diatom comprises Thalassiosira pseudonana. In some embodiments, the centric diatom comprises Thalassiosira weissflogii, In some embodiments, the genus of centric diatoms comprises Cyclotella. In some embodiments, the centric diatom comprises Cyclotella cryptica. In some embodiments, the genus of centric diatoms comprises Chaetoceros. In some embodiments, the centric diatom comprises Chaetoceros muelleri.

In some embodiments, the composition can be added to any pennate diatom, such as Cylindrotheca fusiformis and Phaeodactylum reticulum. In some embodiments, the composition can be added to any species of diatom. In some embodiments, the composition can be added to any species of unicellular microalgae.

In some embodiments, the composition is administered to shrimp, for example, in the form of shrimp feed. In some embodiments, the composition is administered to another animal, such as any one or more of fish, poultry, swine, cattle, sheep, and goats, for example, in the form of animal feed. Illustratively, the composition may be administered to fish, such as Tilapia fish, in the form of fish feed.

In some embodiments, the composition is used in a method to treat shrimp. In some embodiments, the composition is used to treat other animals such as any one or more of fish, poultry, swine, cattle, sheep, and goats. In some embodiments, the composition is used to treat Tilapia fish.

In some embodiments, any of the compositions, fusion proteins, and adjuvantic proteins described herein is combined with any of the animal feeds described herein.

In an aspect, a composition is provided comprising a sequence having at least 80% sequence identity with any of SEQ ID NOs: 3, 12, 13, 14, 15, 16, 17, 18, and 19.

In some embodiments, the sequence has at least 85%, at least 90%, or at least 95%, sequence identity with SEQ ID NO: 3. In embodiments, the sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 3. In some embodiments, the sequence comprises SEQ ID NO: 3.

In some embodiments, the sequence has at least 85%, at least 90%, or at least 95%, sequence identity with SEQ ID NO: 12. In some embodiments, the sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 12. In some embodiments, the sequence comprises SEQ ID NO: 12.

In some embodiments, the sequence has at least 85%, at least 90%, or at least 95%, sequence identity with SEQ ID NO: 13. In embodiments, the sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 13. In some embodiments, the sequence comprises SEQ ID NO: 13.

In some embodiments, the sequence has at least 85%, at least 90%, or at least 95%, sequence identity with SEQ ID NO: 14. In embodiments, the sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 14. In some embodiments, the sequence comprises SEQ ID NO: 14.

In some embodiments, the sequence has at least 85%, at least 90%, or at least 95%, sequence identity with SEQ ID NO: 15. In embodiments, the sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 15. In some embodiments, the sequence comprises SEQ ID NO: 15.

In some embodiments, the sequence has at least 85%, at least 90%, or at least 95%, sequence identity with SEQ ID NO: 16. In embodiments, the sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 16. In some embodiments, the sequence comprises SEQ ID NO: 16.

In some embodiments, the sequence has at least 85%, at least 90%, or at least 95%, sequence identity with SEQ ID NO: 17. In embodiments, the sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 17. In some embodiments, the sequence comprises SEQ ID NO: 17.

In some embodiments, the sequence has at least 85%, at least 90%, or at least 95%, sequence identity with SEQ ID NO: 18. In embodiments, the sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 18. In some embodiments, the sequence comprises SEQ ID NO: 18.

In some embodiments, the sequence has at least 85%, at least 90%, or at least 95%, sequence identity with SEQ ID NO: 19. In embodiments, the sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 19. In some embodiments, the sequence comprises SEQ ID NO: 19.

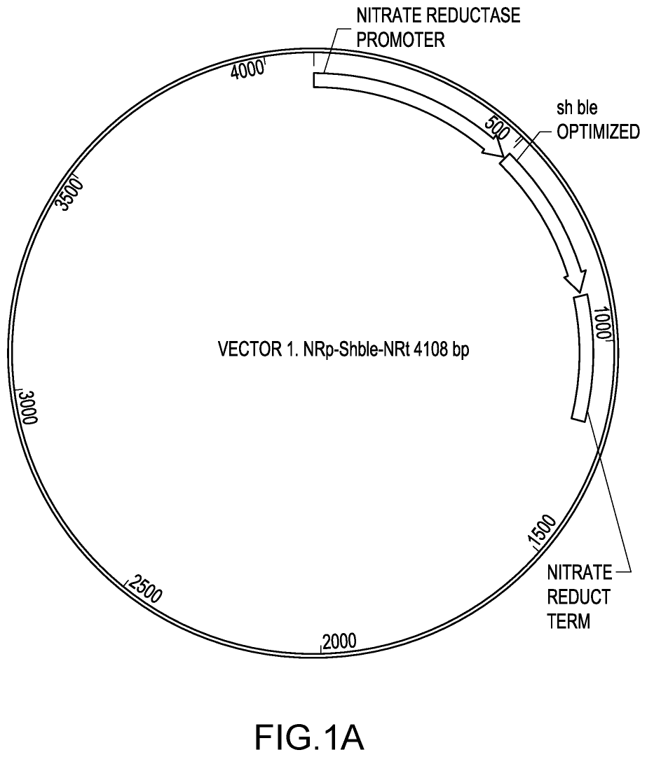

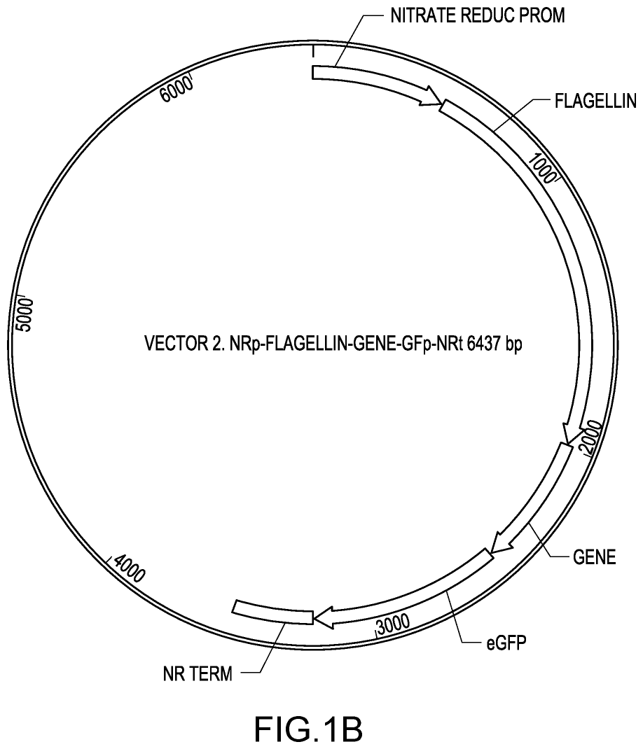

In some embodiments, the composition is part of a fusion protein. In embodiments, the fusion protein is derived from either of the vectors shown in FIG. 1A or 1B and described in greater detail in the Working Examples. In embodiments, the flagellin sequence shown in FIG. 1B comprises a naturally occurring flagellin sequence. In embodiments, the flagellin sequence shown in FIG. 1B comprises a codon-optimized flagellin sequence.

In an aspect, a composition is provided comprising a sequence that has at least 80% sequence identity with SEQ ID NO: 4.

In some embodiments, the sequence has at least 85%, at least 90%, or at least 95%, sequence identity with SEQ ID NO: 4. In embodiments, the sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 4. In some embodiments, the sequence comprises SEQ ID NO: 4.

In some embodiments, the composition is part of a fusion protein. In embodiments, the fusion protein is derived from the vectors shown in FIGS. 1A and 1B. In some embodiments, the fusion protein shown in FIG. 1B comprises a naturally occurring flagellin sequence.

In an aspect, a composition is provided, comprising at least one protein derived from a microbe. In some embodiments, the at least one protein derived from a microbe comprises flagellin. In embodiments, the at least one protein derived from a microbe comprises a naturally occurring flagellin sequence. In embodiments, the at least one protein derived from a microbe comprises a synthetic flagellin sequence. In some embodiments, the synthetic flagellin sequence comprises a codon-optimized flagellin sequence. In some embodiments, the microbe comprises a bacterium.

In some embodiments, the flagellin comprises a sequence having at least 80% sequence identity with SEQ ID NO: 4. In embodiments, the sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 4. In some embodiments, the sequence comprises SEQ ID NO: 4

In some embodiments, the flagellin comprise a sequence having at least 80% sequence identity with SEQ ID NO: 3. In embodiments, the sequence has at least 85%, at least 90%, at least 95%, sequence identity with SEQ ID NO: 3. In embodiments, the sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 95%, approximately 92%, approximately 93%, approximately 94%, approximately 96%, approximately 97%, approximately 98%, or approximately 99% sequence identity with SEQ ID NO: 3. In some embodiments, the sequence comprises SEQ ID NO: 3.

In an aspect, a composition is provided comprising an adjuvantic protein. In some embodiments, the adjuvantic protein comprises a heterologous protein comprising flagellin. In some embodiments, the heterologous protein comprises flagellin and any one or more of an antigen, a hormone, a toxin, and an antibody.

In an aspect, a composition is provided comprising an adjuvantic protein that is expressed in a centric diatom. In some embodiments, the centric diatom is any centric diatom described herein. In some embodiments, the adjuvantic protein is expressed in any one or more of the cytoplasm, the plasma membrane, the chloroplast, the endoplasmic reticulum, and the chloroplast endoplasmic reticulum, of the centric diatom. In some embodiments, the adjuvantic protein is expressed in any subcellular compartment of the centric diatom.

In some embodiments, the centric diatom comprises Thalassiosira pseudonana, Thalassiosira weissflogii, Cyclotella cryptica, or Chaetoceros muelleri. In some embodiments, the centric diatom comprises any diatom known in the art.

### Methods

In an aspect, a method of treating an animal is provided comprising adding at least one protein from a microbe to a diatom cell; and administering the diatom cell to the animal. In some embodiments, the animal is any one or more of fish, poultry, swine, cattle, sheep, and goats. In some embodiments, the animal is shrimp. In some embodiments, the animal is fish. In some embodiments, the fish is Tilapia fish. In some embodiments, the at least one protein from a microbe is a flagellin protein.

In another aspect, a method of feeding an animal is provided comprising adding at least one protein from a microbe to a diatom cell; and feeding the diatom cell to the animal. In some embodiments, the animal is any one or more of fish, poultry, swine, cattle, sheep, and goats. In some embodiments, the animal is shrimp. In some embodiments, the animal is fish. In some embodiments, the fish is Tilapia fish. In some embodiments, the at least one protein derived from a microbe is a flagellin protein

As used herein, by “adding at least one protein from a microbe to a diatom cell” it is meant that the at least one protein from a microbe is disposed within the diatom cell in any suitable manner. For example, adding a flagellin to a diatom cell means that the flagellin is exogenously added to the diatom cell in any suitable manner. Illustratively, in some embodiments, the at least one protein from a microbe that is exogenously added to the diatom cell is expressed in the cytoplasm of the diatom cell. In some embodiments, the at least one protein from a microbe that is exogenously added to the diatom cell is expressed in the plasma membrane of the diatom cell. In some embodiments, the at least one protein from a microbe that is exogenously added to the diatom cell is expressed in the chloroplast of the diatom cell. In some embodiments, the at least one protein from a microbe that is exogenously added to the diatom cell is expressed in the endoplasmic reticulum of the diatom cell. In some embodiments, the at least one protein from a microbe that is exogenously added to the diatom cell is expressed in the chloroplast endoplasmic reticulum of the diatom cell. In some embodiments, the at least one protein from a microbe that is exogenously added to the diatom cell is expressed in any subcellular compartment of the diatom cell.

In some embodiments, administering the diatom cell or diatom cell extracts to the animal comprises orally delivering the diatom cell to the animal. In some embodiments, orally delivering the diatom cell or diatom cell extracts comprises using a tablet to orally deliver the diatom cell or diatom cell extracts. In some embodiments, orally delivering the diatom cell or diatom cell extracts comprises using a capsule to orally deliver the diatom cell or diatom cell extracts. In some embodiments, the diatom cell or diatom cell extracts is delivered through any pharmaceutically viable dosage form described herein.

In some embodiments, administering the diatom cell to the animal comprises injecting the diatom cell or diatom cell extracts into the animal. In some embodiments, injecting the diatom cell into the animal comprises intravenously injecting the diatom cell or diatom cell extracts into the animal. In some embodiments, injecting the diatom cell into the animal comprises intramuscularly injecting the diatom cell or diatom cell extracts into the animal. In some embodiments, injecting the diatom cell into the animal comprises subcutaneously injecting the diatom cell or diatom cell extracts into the animal. In some embodiments, injecting the diatom cell into the animal comprises intradermally injecting the diatom cell or diatom cell extracts into the animal. In some embodiments, administering the diatom cell or diatom cell extracts to the animal is performed through any pharmaceutically acceptable method of administration described herein.

In some embodiments, the diatom cell or diatom cell extracts is formulated in a pharmaceutically acceptable dosage form. In some example, the dosage form comprises any dosage form described herein.

In some embodiments, the method comprises administering animal feed to the animals. In some embodiments, the animal feed is shrimp animal feed. In some embodiments, the shrimp animal feed is any shrimp animal feed described herein. In some embodiments, the animal feed is fish animal feed. In some embodiments, the fish animal feed is any fish animal feed described herein.

In some embodiments, a diatom is mixed with the animal feed and then administered or fed to the animal. In some embodiments, the diatom or diatom cell extracts is mixed with shrimp animal feed. In some embodiments, the diatom or diatom cell extracts is mixed with fish animal feed. In some embodiments, the diatom or diatom cell extracts is mixed with the animal feed during an industrial manufacturing process. In some embodiments, the industrial manufacturing process comprises mixing the diatom or diatom cell extracts with other feed component(s). In some embodiments, the diatom or diatom cell extracts is added to the animal feed using infusion or top-coating with (carboxymethyl cellulose (CMC), corn starch (CS) and wheat glucan (WG). In some embodiments, the diatom or diatom cell extracts is added to the animal feed using spray coating. In some embodiments, the diatom or diatom cell extracts is added to the animal feed using top coating with stearin oil or fish oil including Tuna and Menhaden oil. In some embodiments, mixing the diatom or diatom cell extracts with the animal feed comprises the use of a binding agent. In some embodiments, mixing the diatom or diatom cell extracts with the animal feed is performed without the use of a binding agent. In some embodiments, the diatom or diatom cell extracts that is mixed with the animal feed is any diatom described herein.

In some embodiments, the at least one protein from a microbe comprises flagellin. In some embodiments, the microbe is Salmonella enterica, Bacillus subtilis, Vibrio anguillarum, Pseudomonas aeruginosa, Salmonella typhimurium, Serratia marcescens, Edwardsiella tarda, Proteus mirabilis, Escherichia coli, Shigella flexneri, Listeria monocytogenes, Bartonella bacilliformis, Rhizobium meliloti, Campylobacter jejuni, Campylobacter coli, Helicobacter pylori, Helicobacter felis, Helicobacter hepaticus, Legionella pneumophila, Salmonella marcescens, or Wolinella succinogenes. Other examples of microbies that comprise flagellin are referenced in Andersen-Nissen E. et al. 2005. “Evasion of Toll-like receptor 5 by flagellated bacteria.” Proc. Natl. Acad. Sci. U.S.A. 102 (26): 9247-9252, which is hereby incorporated by reference in its entirety. In some embodiments, the flagellin protein is any naturally occurring flagellin protein or any synthetic flagellin protein described herein. In some embodiments, the flagellin sequence comprises any one or more of SEQ ID Nos: 3, 4, and 12-19. In some embodiments the flagellin sequence comprises 80% identity of any one or more of SEQ ID Nos: 3, 4, and 12-19.

In some embodiments, the flagellin sequence has at least 85%, at least 90%, or at least 95%, sequence identity with SEQ ID NO: 3. In embodiments, the flagellin sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 3. In some embodiments, the flagellin sequence comprises SEQ ID NO: 3.

In some embodiments, the flagellin sequence has at least 85%, at least 90%, or at least 95%, sequence identity with SEQ ID NO: 4. In embodiments, the flagellin sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 4. In some embodiments, the flagellin sequence comprises SEQ ID NO: 4

In some embodiments, the flagellin sequence has at least 85%, at least 90%, or at least 95%, sequence identity with SEQ ID NO: 12. In some embodiments, the flagellin sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 12. In some embodiments, the flagellin sequence comprises SEQ ID NO: 12.

In some embodiments, the flagellin sequence has at least 85%, at least 90%, or at least 95%, sequence identity with SEQ ID NO: 13. In embodiments, the flagellin sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 13. In some embodiments, the flagellin sequence comprises SEQ ID NO: 13.

In some embodiments, the flagellin sequence has at least 85%, at least 90%, or at least 95%, sequence identity with SEQ ID NO: 14. In embodiments, the flagellin sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 14. In some embodiments, the flagellin sequence comprises SEQ ID NO: 14.

In some embodiments, the flagellin sequence has at least 85%, at least 90%, or at least 95%, sequence identity with SEQ ID NO: 15. In embodiments, the flagellin sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 15. In some embodiments, the flagellin sequence comprises SEQ ID NO: 15.

In some embodiments, the flagellin sequence has at least 85%, at least 90%, or at least 95%, sequence identity with SEQ ID NO: 16. In embodiments, the flagellin sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 16. In some embodiments, the flagellin sequence comprises SEQ ID NO: 16.

In some embodiments, the flagellin sequence has at least 85%, at least 90%, or at least 95%, sequence identity with SEQ ID NO: 17. In embodiments, the flagellin sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 17. In some embodiments, the composition comprises SEQ ID NO: 17.

In some embodiments, the flagellin sequence has at least 85%, at least 90%, or at least 95%, sequence identity with SEQ ID NO: 18. In embodiments, the flagellin sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 18. In some embodiments, the flagellin sequence comprises SEQ ID NO: 18.

In some embodiments, the flagellin sequence has at least 85%, at least 90%, or at least 95%, sequence identity with SEQ ID NO: 19. In embodiments, the flagellin sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 19. In some embodiments, the flagellin sequence comprises SEQ ID NO: 19.

In an aspect, a treatment is provided comprising administering any of the compositions described herein to an animal. In some embodiments, the animal is shrimp. In some embodiments, the animal is fish, poultry, swine, or cattle. In some embodiments, the fish is a Tilapia fish. In some embodiments, the composition comprises a flagellin sequence. In some the flagellin sequence comprises any one or more of SEQ ID Nos: 3, 4, and 12-19. In some embodiments the flagellin sequence comprises 80% identity of any one or more of SEQ ID Nos: 3, 4, and 12-19.

In some embodiments, the flagellin sequence has at least 85%, at least 90%, or at least 95%, sequence identity with SEQ ID NO: 3. In embodiments, the flagellin sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 3. In some embodiments, the flagellin sequence comprises SEQ ID NO: 3.

In some embodiments, the flagellin sequence has at least 85%, at least 90%, or at least 95%, sequence identity with SEQ ID NO: 4. In embodiments, the flagellin sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 4. In some embodiments, the flagellin sequence comprises SEQ ID NO: 4

In some embodiments, the flagellin sequence has at least 85%, at least 90%, or at least 95%, sequence identity with SEQ ID NO: 12. In some embodiments, the flagellin sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 12. In some embodiments, the flagellin sequence comprises SEQ ID NO: 12.

In some embodiments, the flagellin sequence has at least 85%, at least 90%, or at least 95%, sequence identity with SEQ ID NO: 13. In embodiments, the flagellin sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 13. In some embodiments, the flagellin sequence comprises SEQ ID NO: 13.

In some embodiments, the flagellin sequence has at least 85%, at least 90%, or at least 95%, sequence identity with SEQ ID NO: 14. In embodiments, the flagellin sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 14. In some embodiments, the flagellin sequence comprises SEQ ID NO: 14.

In some embodiments, the flagellin sequence has at least 85%, at least 90%, or at least 95%, sequence identity with SEQ ID NO: 15. In embodiments, the flagellin sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 15. In some embodiments, the flagellin sequence comprises SEQ ID NO: 15.

In some embodiments, the flagellin sequence has at least 85%, at least 90%, or at least 95%, sequence identity with SEQ ID NO: 16. In embodiments, the flagellin sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 16. In some embodiments, the flagellin sequence comprises SEQ ID NO: 16.

In some embodiments, the flagellin sequence has at least 85%, at least 90%, or at least 95%, sequence identity with SEQ ID NO: 17. In embodiments, the flagellin sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 17. In some embodiments, the flagellin sequence comprises SEQ ID NO: 17.

In some embodiments, the flagellin sequence has at least 85%, at least 90%, or at least 95%, sequence identity with SEQ ID NO: 18. In embodiments, the flagellin sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 18. In some embodiments, the flagellin sequence comprises SEQ ID NO: 18.

In some embodiments, the flagellin sequence has at least 85%, at least 90%, or at least 95%, sequence identity with SEQ ID NO: 19. In embodiments, the sequence comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 19. In some embodiments, the composition comprises SEQ ID NO: 19

In some embodiments, the treatment comprises a therapeutic treatment. In some embodiments, the treatment comprises a prophylactic treatment.

In some embodiments, the diatom comprises algae. In embodiments, the diatom comprises microalgae. In some embodiments, the diatom comprises Thalassiosira pseudonana, Thalassiosira weissflogii, Cyclotella cryptica, or Chaetoceros muelleri. In some embodiments, the diatom comprises a diatom from any one of the genera Thalassiosira, Cyclotella, and Chaetoceros.

In an aspect, a method is provided comprising expressing an adjuvantic protein in one or more centric diatoms. In some embodiments, the one or more centric diatoms comprises any one or more of Thalassiosira pseudonana, Thalassiosira weissflogii, Cyclotella cryptica, and Chaetoceros muelleri. In some embodiments, the diatom comprises a diatom from any one of the genera Thalassiosira, Cyclotella, and Chaetoceros. In some embodiments, the adjuvantic protein comprises any fusion protein described herein. In some embodiments, the fusion protein comprises flagellin. In some embodiments, the fusion protein comprises flagellin fused to any one or more of an antigen, a hormone, a toxin, or an antibody.

In some embodiments, the adjuvantic protein is expressed in any one or more of the cytoplasm, the chloroplast, the plasma membrane, and the endoplasmic reticulum. In some embodiments, the adjuvantic protein is expressed in any subcellular compartment of the centric diatom.

In an aspect, a method of feeding an animal is provided, the method comprising feeding a diatom or diatom cell extracts to the animal, wherein the diatom expresses at least one exogenous flagellin protein. In some examples, the feeding may administer the diatom or diatom cell extracts to the animal (for example, in a therapeutically effective amount).

In some embodiments, the animal is fish. In some embodiments, fish is Tilapia fish. In some embodiments, the animal is shrimp.

In embodiments, the at least one flagellin protein is exogenously expressed in the cytoplasm of the diatom. In embodiments, the at least one flagellin protein is exogenously expressed in the in the plasma membrane of the diatom. In embodiments, the at least one flagellin protein is exogenously expressed in the chloroplast of the diatom. In embodiments, the at least one flagellin protein is exogenously expressed in the endoplasmic reticulum of the diatom. In embodiments, the at least one flagellin protein is exogenously expressed in the chloroplast endoplasmic reticulum. In embodiments, the at least one flagellin protein is exogenously expressed in any subcellular compartment of the diatom.

In some embodiments, the at least one flagellin protein comprises SEQ ID NO: 4. In some embodiments, the flagellin protein has at least 80%, at least 85%, at least 90%, or at least 95% sequence identity with SEQ ID NO: 4. In some embodiments, the flagellin protein comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with SEQ ID NO: 4.

In some embodiments, the at least one flagellin protein comprises at least one codon optimized flagellin protein. In some embodiments, the at least one codon optimized protein comprises any one or more of SEQ ID NOs: 3, 12, 13, 14, 15, 16, 17, 18, and 19. In some embodiments, the at least codon optimized protein comprises a sequence that has at least 80%, at least 85%, at least 90%, or at least 95% sequence identity with any one or more of SEQ ID NOs: 3, 12, 13, 14, 15, 16, 17, 18, and 19. In some embodiments, the at least one codon optimized protein comprises approximately 80%, approximately 81%, approximately 82%, approximately 83%, approximately 84%, approximately 85%, approximately 86%, approximately 87%, approximately 88%, approximately 89%, approximately 90%, approximately 91%, approximately 92%, approximately 93%, approximately 94%, approximately 95%, approximately 96%, approximately 97%, approximately 98%, or approximately 99% sequence identity with any one or more of SEQ ID NOs: 3, 12, 13, 14, 15, 16, 17, 18, and 19.

In some embodiments, the diatom comprises Thalassiosira pseudonana, Thalassiosira weissflogii, Cyclotella cryptica, or Chaetoceros muelleri. In some embodiments, the diatom comprises any diatom known in the art.

### Dosage and Dosage Forms

The disclosed compositions and cells allow for short, medium, or long-term expression of genes or sequences of interest. Accordingly, dosing may vary based upon the condition being treated and the method of administration.

In embodiments, compositions and cells may be administered to an animal in need in varying doses. Additionally, vector compositions and cells of the present disclosure may be administered periodically, such as once or twice a day, or any other suitable time period. For example, compositions and cells may be administered to a subject in need once a week, once every other week, once every three weeks, once a month, every other month, every three months, every six months, every nine months, once a year.

In embodiments, the disclosed compositions and cells are administered as a pharmaceutical composition. Each of the dosage forms can comprise various solubilizing agents, disintegrating agents, surfactants, fillers, thickeners, binders, diluents such as wetting agents or other pharmaceutically acceptable excipients. The pharmaceutical composition can also be formulated for injection, insufflation, infusion, or intradermal exposure. For instance, an injectable formulation may comprise the disclosed vectors in an aqueous or non-aqueous solution at a suitable pH and tonicity.

The disclosed compositions and cells can be administered using any pharmaceutically acceptable method to the animal, such as intranasal, buccal, sublingual, oral, rectal, ocular, parenteral (intravenously, intradermally, intramuscularly, subcutaneously, intraperitoneally), pulmonary, intravaginal, locally administered, topically administered, topically administered after scarification, mucosally administered, via an aerosol, in semi-solid media such as agarose or gelatin, or via a buccal or nasal spray formulation.

Further, the disclosed compositions and cells can be formulated into any pharmaceutically acceptable dosage form, such as a solid dosage form, tablet, pill, lozenge, capsule, liquid dispersion, gel, aerosol, pulmonary aerosol, nasal aerosol, ointment, cream, semi-solid dosage form, a solution, an emulsion, and a suspension. Further, the pharmaceutical composition may be a controlled release formulation, sustained release formulation, immediate release formulation, or any combination thereof. Further, the pharmaceutical composition may be a transdermal delivery system.

In embodiments, the pharmaceutical composition can be formulated in a liquid dosage form for oral administration, such as suspensions, emulsions or syrups. In embodiments, the liquid dosage form can include, in addition to commonly used simple diluents such as water and liquid paraffin, various excipients such as humectants, sweeteners, aromatics or preservatives. In embodiments, the pharmaceutical composition can be formulated in a solid dosage form for oral administration, and the solid dosage form can be powders, granules, capsules, tablets or pills. The dosage of the pharmaceutical composition can vary depending on the animal's weight, age, gender, administration time and mode, excretion rate, and/or the severity of the disease.

### Examples

The following examples are intended to be purely illustrative, and not limiting of the present disclosure.

### Example 1. Vector Construction

Vector 1. NRp—Sh ble-NRt (FIG. 1A) (SEQ ID NO: 11)

The nitrate reductase (NR) promoter, which enables expression in the presence of nitrate and repression by ammonium. The NR promoter was developed for the pennate diatom Cylindrotheca fusiformis, and subsequently adapted for other diatoms, including Thalassiosira pseudonana and Phaeodactylum tricornutum. The NR promoter and terminator DNA sequences were obtained from the Thalassiosira pseudonana genome database (mycocosm.jgi.doe.gov/Thaps3/Thaps3.home.html).

Streptoalloteichus hindustanus gene (Sh ble) encodes a protein that confers resistance to the antibiotics Zeocin and phleomycin. The Sh ble sequence (GenBank accession no. WP_230684036.1) was codon optimized according to Thalassiosira pseudonana codon usage data from the Kazusa DNA Research Institute (www.kazusa.or.jp/codon/cgi-bin/showcodon.cgi?species=35128).

A DNA sequence encoding NR promoter, Sh ble and NR terminator DNA sequence was synthesized and cloned into pUC57.

Vector 2. NRp—Gene—eGFP—NRt (FIG. 1B) (SEQ ID NO: 12)

Flagellins, the structural proteins of the bacterial flagellum, are shown to be potent immunostimulants and vaccine adjuvants, which activate several immune and non-immune cells, including T, B, dendritic cells, natural killer (NK), and non-lymphoid cells through Toll-like receptor 5-TLR5.

To increase the immunogenicity of the subunit vaccine, a N-terminus flagellin was cloned at 5′ end of the antigen-of-interest that serves as a built-in immunostimulant and adjuvant. FliC/FljB family flagellin [Salmonella enterica subsp. enterica serovar Typhimurium] (GenBank accession no. ECX1283599.1) was codon optimized according to Thalassiosira pseudonana codon usage data from the Kazusa DNA Research Institute (www.kazusa.or.jp/codon/cgi-bin/showcodon.cgi?species=35128).

Among several selected antigen/immunomodulator candidates, an outer membrane protein Slp/YeaY family lipoprotein [Vibrio parahaemolyticus] (GenBank accession no. MBD6991300.1) was codon optimized according to Thalassiosira pseudonana codon usage data from the Kazusa DNA Research Institute (www.kazusa.or.jp/codon/cgi-bin/showcodon.cgi?species=35128).

### Example 2. Transformation of the Vector into the Centric Diatoms

Thalassiosira pseudonana, Thalassiosira wesiflogii (Conticribra weissflogii) and Cyclotella cryptica. Two plasmid vectors (Vector 1: SEQ ID NO: 11; FIG. 1A) (Vector 2: SEQ ID NO: 12; FIG. 1B), first expressing the resistance marker Sh ble and second expressing adjuvant-antigen fusion proteins, were co-transformed into diatom cells by the particle bombardment method. Briefly, exponentially grown the centric diatoms Thalassiosira pseudonana, T. weissflogii, Cyclotella cryptica and Chaetoceros muelleri cells were harvested, and 1×10⁸ cells were plated in a 5 cm diameter circle in the middle of the F/2-agar plate lacking antibiotics. The transformation was then performed by bombarding plasmid DNA-coated tungsten beads (M-17, 1.1 μm diameter, BioRad) onto cells plated on the agar plate under vacuum at a distance of 8 cm at 1350 psi. Plates were bombarded twice to obtain higher transformation efficiency. Immediately after bombardment, cells were overlaid with 10 mL of F/2 and placed under light for 24 h. The cells were then plated on the F/2-agar containing 1 μg/mL zeocin. The positive clones were further selected by screening C-terminus eGFP under a fluorescent microscope.

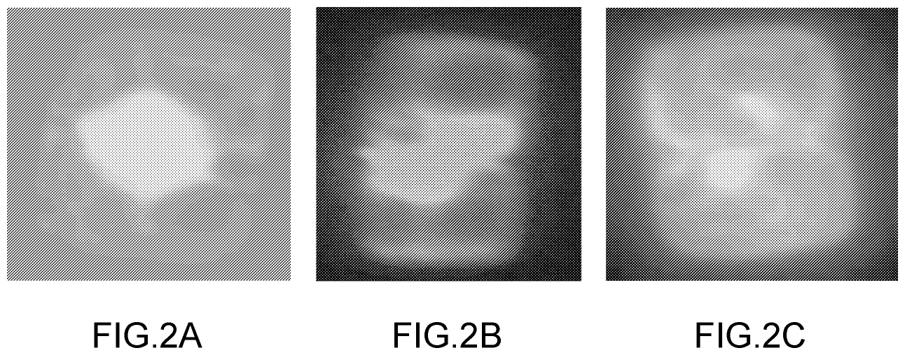

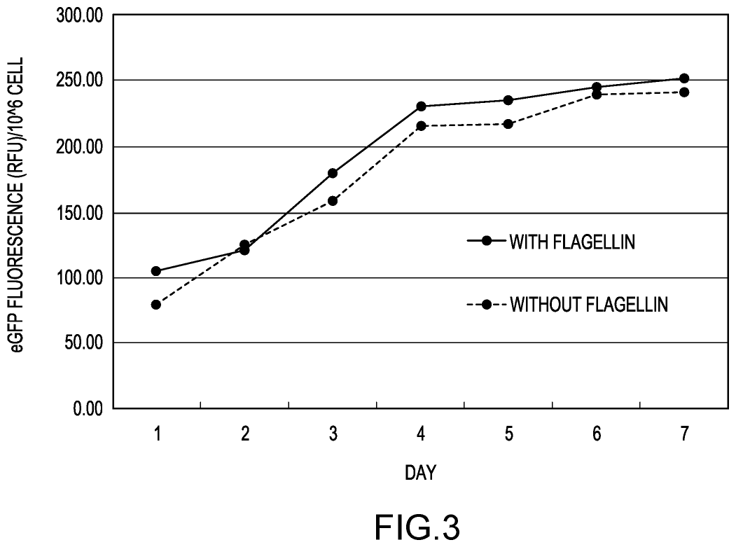

FIGS. 2A-2C show expression of flagellin-Vibrio parahaemolyticus Slp/YeaY family lipoprotein fusion protein in the representative centric diatoms, Thalassiosira pseudonana (FIG. 2A), Thalassiosira weissflogii (Conticribra weissflogii) (FIG. 2B), and Cyclotella cryptica (FIG. 2C). In each of FIGS. 2A-2C, the bright white color represents the C-terminus GFP tag. FIG. 3 shows a time-course expression analysis of eGFP-tagged Vp OMP fusion protein with and without flagellin in Thalassiosira pseudonana.

### Example 3. Vaccination/Immune Stimulation Trial in Shrimp

Challenge trial. The diatom cell biomass expressing adjuvant-antigen was mixed in a commercial shrimp diet (Grower 45%) purchased from Rangen Inc, Idaho. In the oral vaccination/immune stimulation trial performed at the Aquaculture Pathology Laboratory-University of Arizona, Tucson, shrimps were fed with the control and test diets equivalent to 5% body mass for 7 days, followed by the vaccinated and control shrimps were challenged with the Vibrio parahaemolyticus strain A3 causing Acute Hepatopancreatic Necrosis Disease (AHPND) by immersing the bacterial culture in the shrimp tanks. The optical density (OD600 nm) of the bacterial suspension used for this challenge was 15.546 with 3.77λ10⁹ colony-forming units. Shrimps continued to feed on the same diets for the next 7 days. Dead animals were removed once a day for the duration of the study. The challenge study was terminated on the 7^{th} day, with live animals counted as survivors.

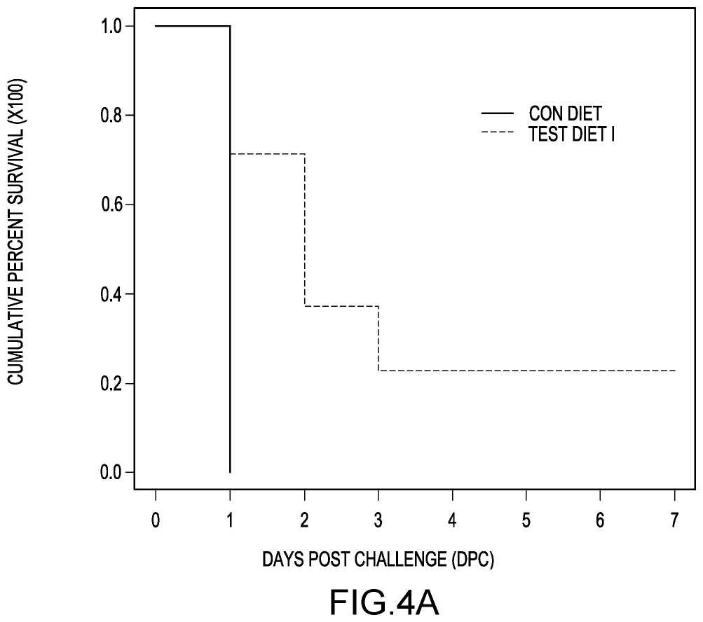

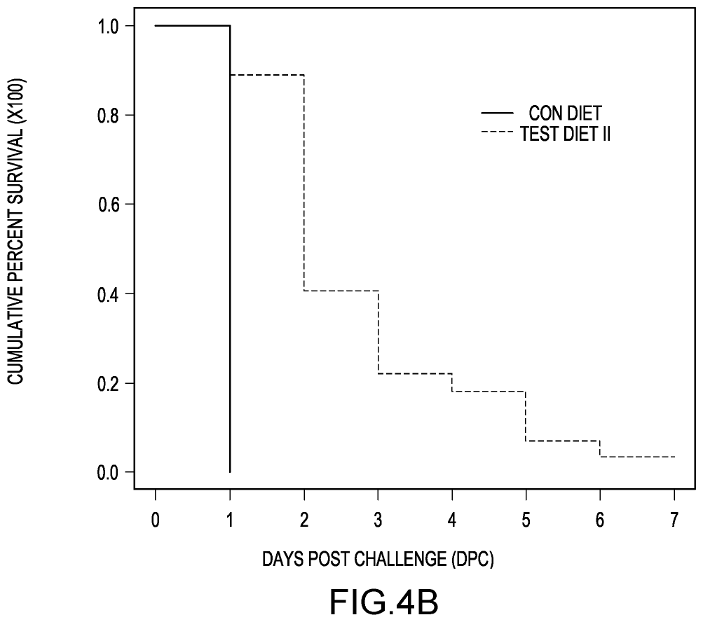

FIG. 4A shows percent survival of juvenile shrimp after they were challenged with Vibrio parahaemolyticus following a 7-day pre-feeding of control (wild type Thalassiosira pseudonana) and test diet flagellin-Vp OMP fusion protein expressed in Thalassiosira pseudonana. All (100%) shrimp fed with the control diet died of the AHPND disease caused by V. parahaemolyticus, whereas 22.5% of shrimps fed with the test diet survived through the test duration of 14 days.

FIG. 4B shows percent survival of juvenile shrimp after they were challenged with Vibrio parahaemolyticus following a 7-day pre-feeding of control (wild type Cylotella cryptica) and test diet flagellin-Vp OMP fusion protein expressed in Thalassiosira weissflogii. All (100%) shrimp fed with the control diet died of the AHPND disease caused by V. parahaemolyticus, whereas 18.75% of shrimps fed with the test diet survived through the test duration of 14 days.

### Example 4. Immune Parameter Test

Using a superoxide dismutase assay (SOD), the effect of test diets on shrimp immune parameters without bacterial challenge. Shrimps were fed for 60 days. On the 61^{st} day, hemolymph was drawn from three shrimps from each treatment. The hemolymph was withdrawn using 1-mL syringes with 100 μL of anticoagulant from the ventral sinus of shrimps above the first abdominal segment. The SOD assay in hemolymph was performed using the Amplite® Colorimetric Superoxide Dismutase (SOD) Assay Kit*Enhanced Sensitivity* (AAT Bioquest, Pleasanton, California).

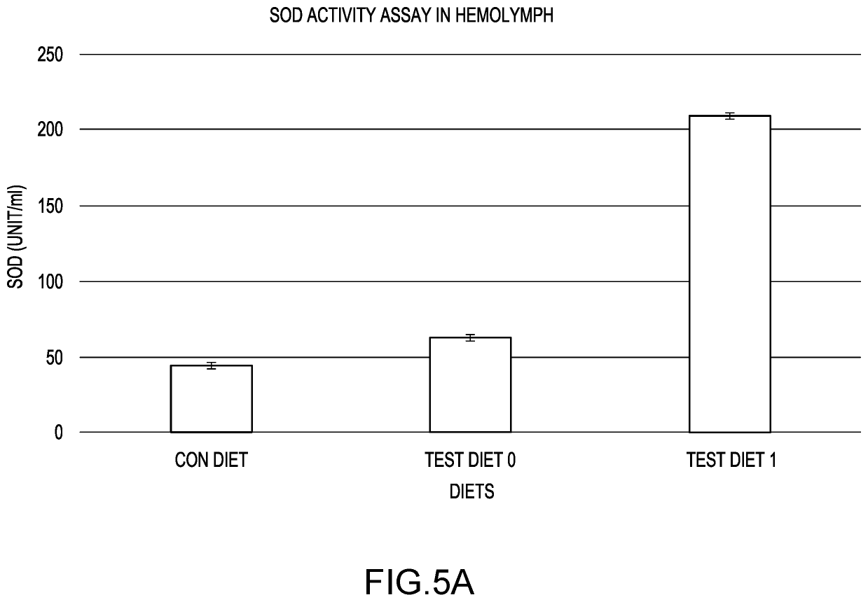

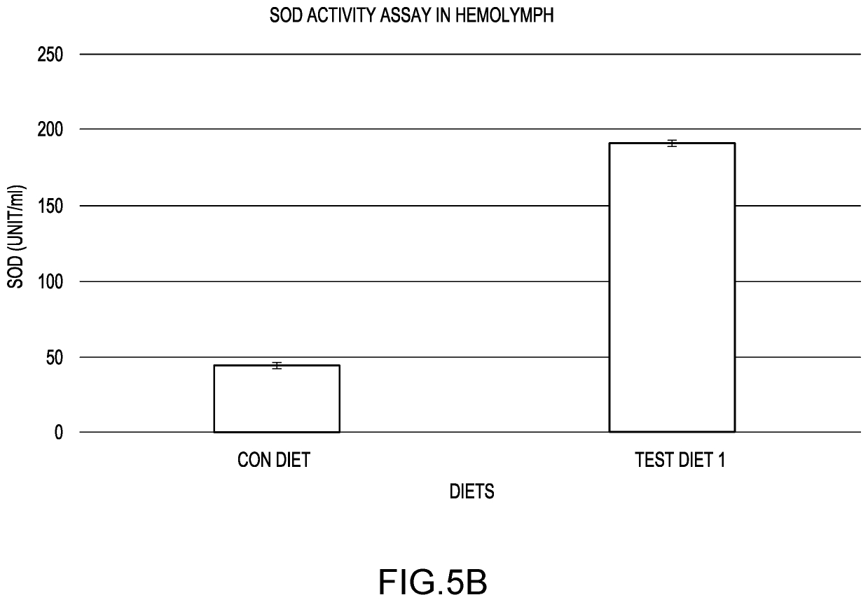

FIG. 5A shows that SOD activity was about four (4) times higher in the hemolymph of shrimps fed with the test diet (flagellin-Vp OMP fusion protein expressed in Thalassiosira pseudonana) than shrimps fed with the control diet (wild type Thalassiosira weissflogii), indicating the diatom expressing immunostimulant induced innate immunity. FIG. 5B shows that SOD activity was about four (4) times higher in the hemolymph of shrimps fed with the test diet (flagellin-Vp OMP fusion protein expressed in Cyclotella cryptica) than shrimps fed with the control diet (wild type Thalassiosira pseudonana), indicating the diatom expressing immunostimulant induced innate immunity.

### Example 5. Using Proteins from Microalgae to Treat Shrimp

In this study treatments of microalgae using various proteins were prepared. The treatments were added to balanced shrimp diets. These treatments provided a significant enhancement and improvement in the shrimp's immune system, as well as their growth and healthy development.

The feed-trial was conducted at L′AQUILA CAMANONERA SHRIMP FARM, located in the province of Oro, Ecuador. Whiteleg shrimp were used in their pre-breeding phase of post-larval age (PL 18-24), at a density of 1,000 animals/m².

The research study was established under a completely randomized design. The analyzed factors included the inclusion of modified proteins at different concentrations and their assessment in balanced diets with the additional modified proteins as a control. The study included three (4) feed treatments (Control, commercial shrimp feed), T1 (commercial shrimp feed incorporated with the 3% pVX_192 diatom biomass), T2 (commercial shrimp feed incorporated with 4% pVX_192 diatom biomass), and T3 (commercial shrimp feed incorporated with 5% pVX_192 diatom biomass)) (see Table 1).

TABLE 1

| | |
|---|---|
| Treatments | Repetitions |

| Control | C1 | C2 | C3 |
|---|---|---|---|
| T1 | T1.1 | T1.2 | T1.3 |
| T2 | T2.1 | T2.2 | T2.3 |
| T3 | T3.1 | T3.2 | T3.3 |

Diet 1 (T1), Diet 2 (T2), Diet 3 (T3), Control
Total of 12 randomly distributed cages; 12,000 organisms

The net cage structures were 1 m² in area and 1.5 m in height covered with a mesh on the interior of the cage and placed on the bottom in the colloid of test shrimp pond floor. The cages were equipped with bottoms stops 30 cm in height, to allow the cages to settle securely on the floor of the pre-breeding shrimp pond. Feeding plates that were 10 cm in diameter were used in each of the twelve (12) cages.

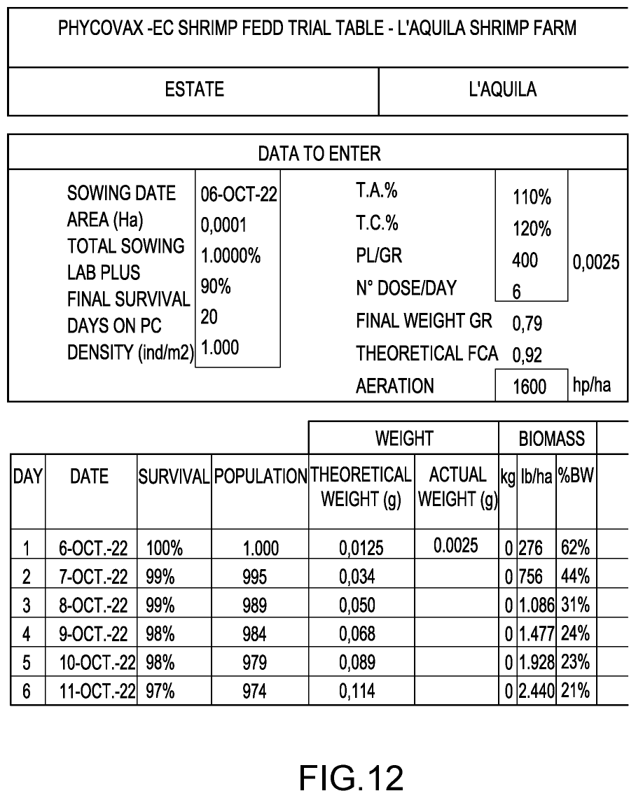

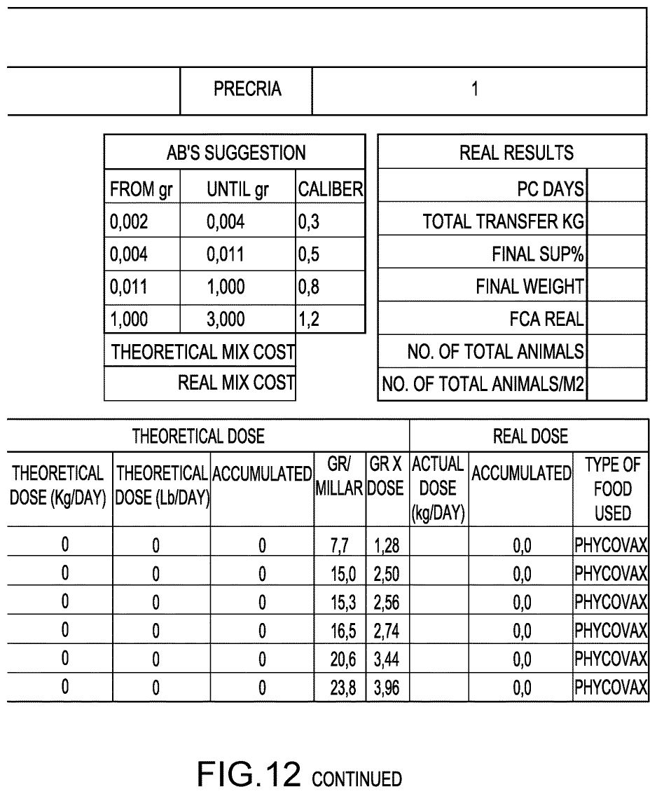

FIG. 12 provides the specifications for each experimental cage enclosure. As shown in FIG. 12, the feed-rations supplied were calculated based on a feed frequency of six (6) times per day from 8:30 am to 8:30 μm. The feeding frequency ensured that the entire pond was managed with continuous aeration to maintain oxygen at appropriate saturation levels, with a replenishment of 2% water each day.

A follow-up was carried out every two (2) days to assess the following parameters:
Morphology metrics: shrimp weight
Animal health: (i) Shrimp survival percentage and (ii) pathology prevalence: the percentage of Vibrio at the beginning and end of each test

In addition, physical chemistry of the pond waters, parameters such as: temperature, pH, salinity, oxygen, and percentage of dissolved oxygen, were recorded daily.

Statistics: This study had a statistical confidence level of 95% and a test performance of 85%. Descriptive statistics were used to determine the mean, standard deviation, and coefficient of variation. The assumptions of the analysis of variance were assessed following normalization of data and homogenization of variance. Corresponding analysis were carried out using ANOVA (analysis of variance, i.e., no relationship between the subjects in each sample). To determine statistical differences between treatments, Tukey's multiple comparison analysis was applied to determine the level of statistical significance between treatments.

Products: The study schedule began by stocking post-larvae, which occurred over the course of approximately seven (7) days. The following day, the test shrimp were harvested so that the organisms could reach their pre-juvenile stage. This allowed the shrimp to provide the results of the study and continue to the next phase of transfer to the fattening stage.

### Results:

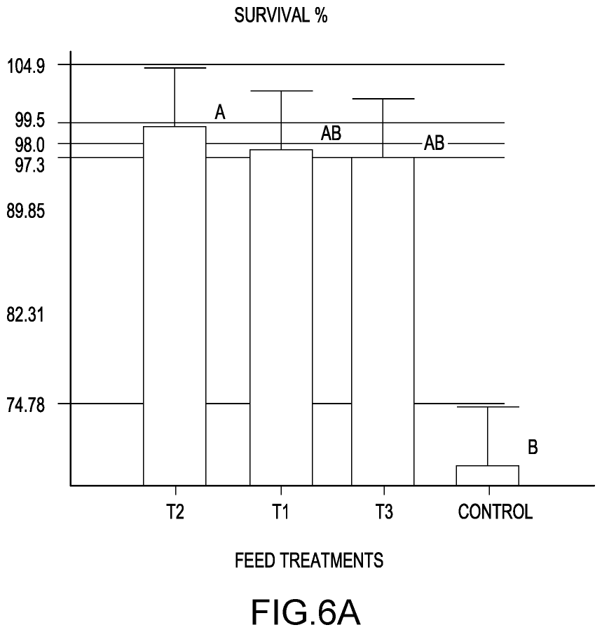

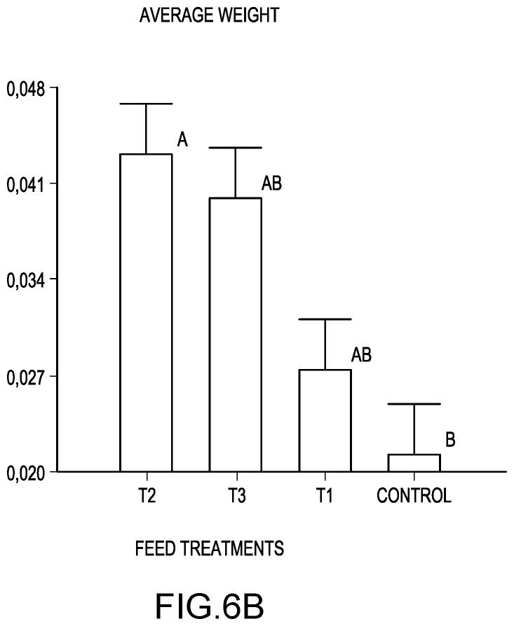

In the shrimp feed trial study, shrimp survival and average weight were evaluated. The results of the study are shown in FIGS. 6A and 6B, which shows the “averaged percentage of survival” of the feed-trial treatments versus the control treatments. The data was analyzed through a Tukey multiple comparison analysis with a confidence level of 95%. This data demonstrates that there was a significant statistical difference between the T2 treatments versus the other treatments and the control.

For each of the treatments (T1, T2, and T3), the shrimp had at least 97% survival rate. See Table 2.

TABLE 2
Feed Treatments and Shrimp Survival Percentages

| | |
|---|---|
| T2 | 99.50% |
| T1 | 98.00% |
| T3 | 97.35% |

For diagnostic purposes, fresh pathology was used to analyze the hepatopancreas of Whiteleg shrimp. During the study, from day one (1), clinical signs and lesions were observed, presenting varying levels of severity with Grade 2 of 20% affectation.

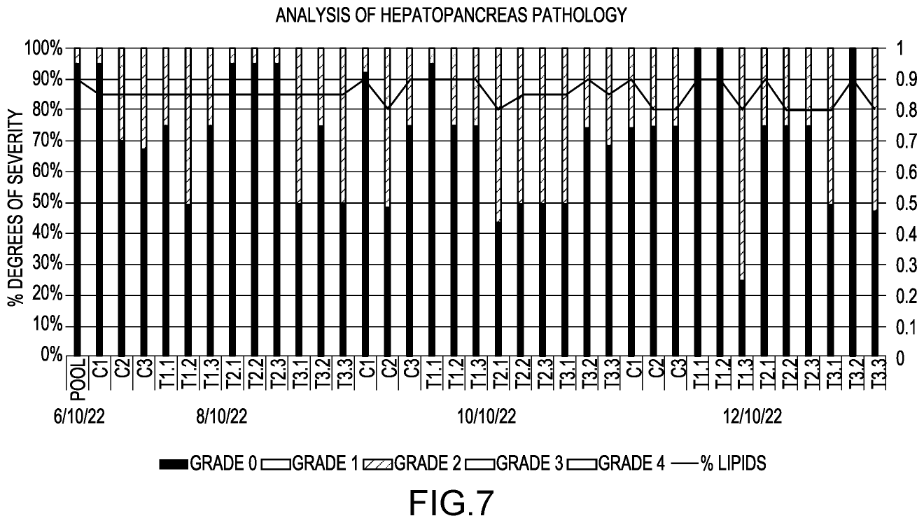

During the study, between treatments, greater effect in T1 and T3 was observed with an average of 50% of moderate Grade 2 levels with respect to the control in which 30% of clinical lesions were observed. Midway through the study, in 30% of the animals, Grade 2 severity was observed, in the animals that had received the T2 or T3 treatments (See FIG. 7). The control had one more lesion at the tubular level with 1% at Grade 3 (see FIG. 7). In the last sampling, T1 and T3 increased their clinical lesion at the level in hepatopancreas at an average of 47% of affectations with Grade 2 of severity (see, FIG. 7).

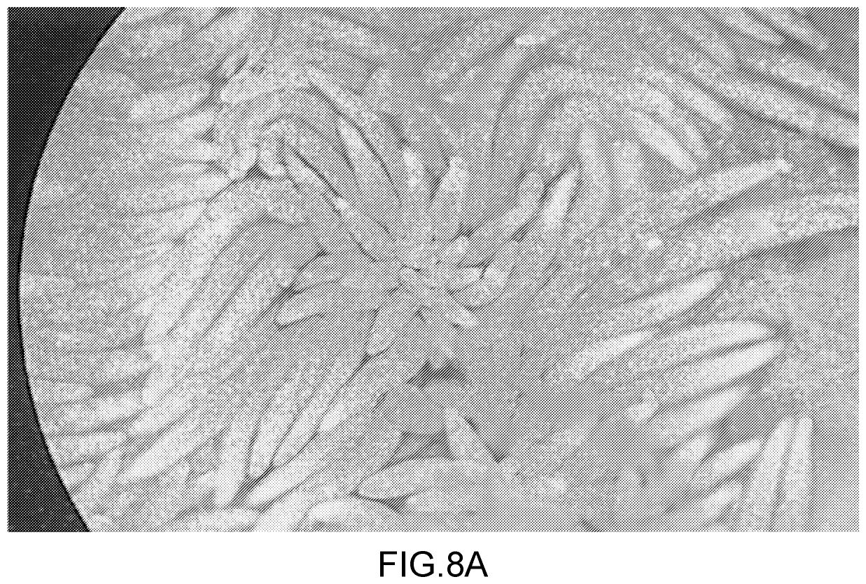

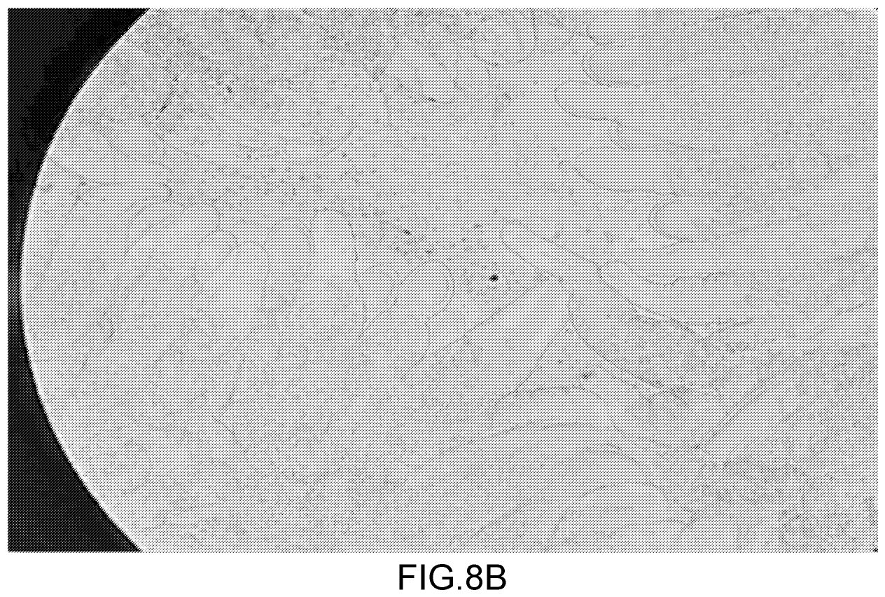

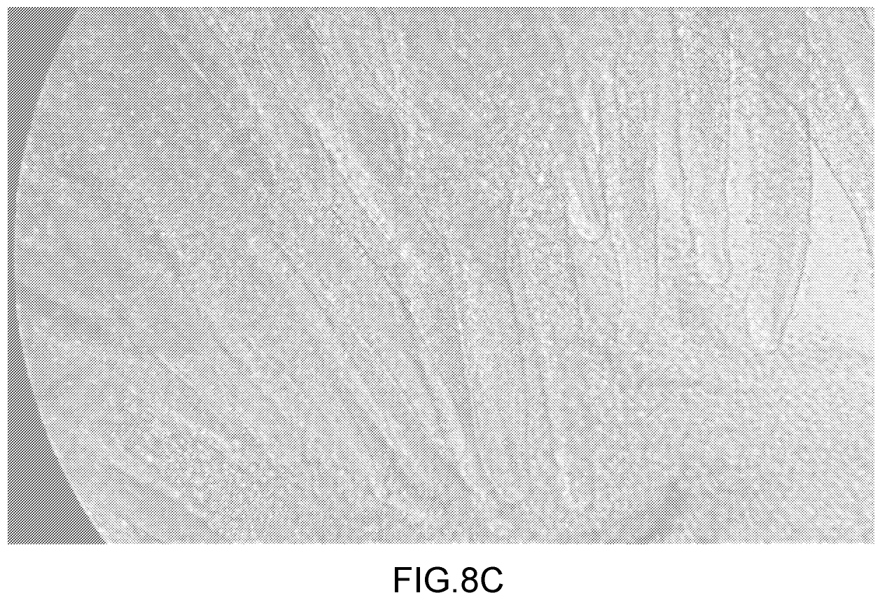

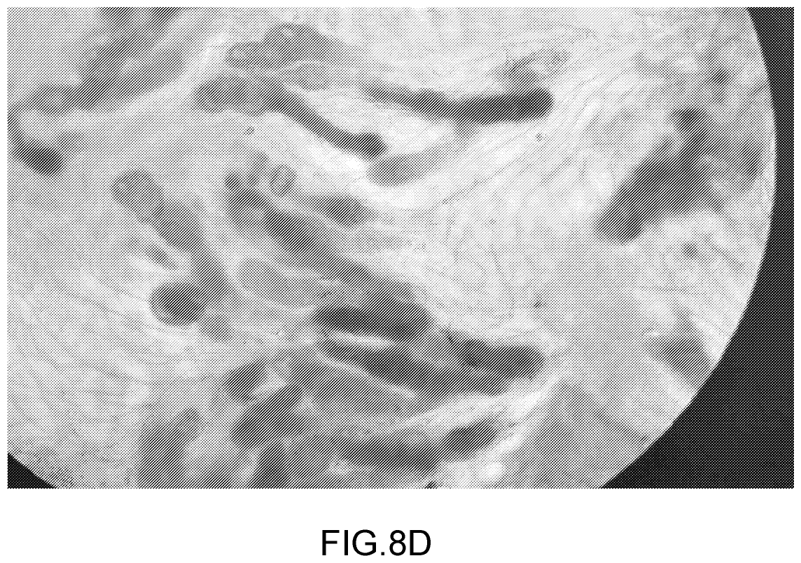

FIG. 8A shows a healthy hepatopancreas. There are no signs of infection or any presence of tubular deformation or roughness. FIG. 8B shows a hepatopancreas with very low presence of tubular deformation. FIG. 8C shows a hepatopancreas with a moderate presence of tubular deformation, atrophy, Melanization, and tubular necrosis. This represents a Phase one (1) infection. Mortality will occur if no treatment is applied. FIG. 8D shows a hepatopancreas with many mishappen tubules observed that contain severe lesions, Melanization, necrosis, and tubular atrophy. This represents a Phase three (3) infection.

This presence of lipid cells in the tubules of the hepatopancreas was also evaluated, which revealed a distinctive pattern of food consumption. Its behavior demonstrated that T1 maintained 87.22% of lipid vacuoles, T2 presented 83.88% of lipid vacuoles, T3 presented 85% of lipid vacuoles, and the control showed 85% of lipid vacuoles.

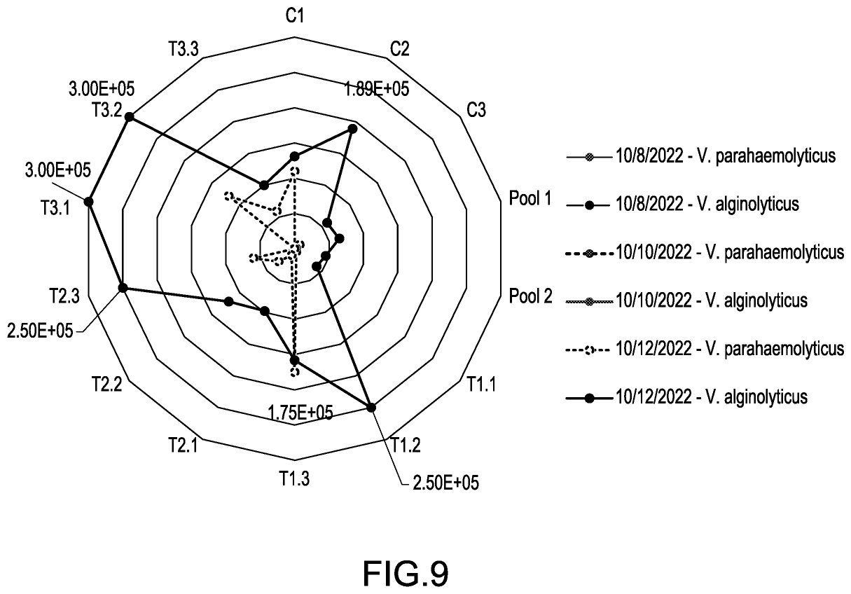

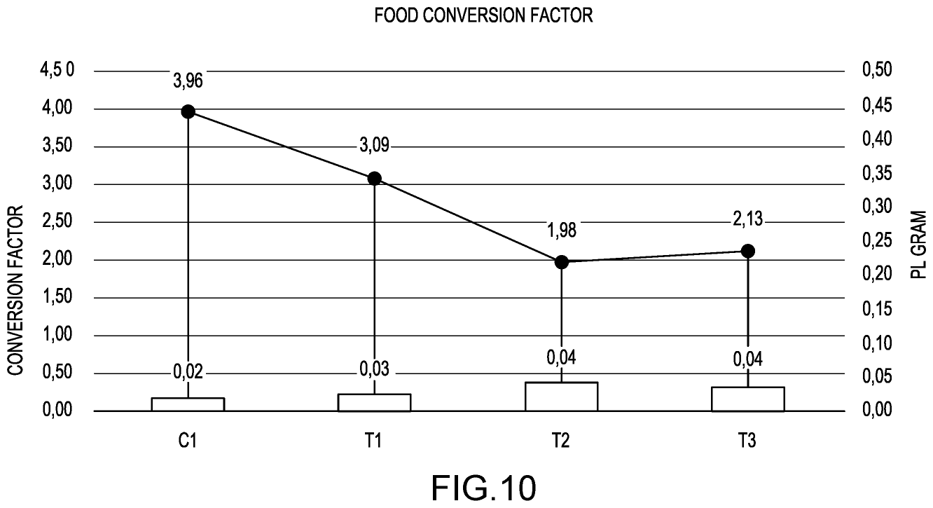

In the microbiological plaque that was carried out during the study, there was bacterial growth of Vibrio alginolyticus colonies of 2.50E+05 cfu/ml, 3.00E+05 cfu/ml (see, FIG. 9). With respect to Vibro Parahaemolytics, the T1 treatment showed colonies 1.75E+05 cfu/ml. The T1, T2, and T3 treatments are those with the Vibrio alginolyticus control of 1.89E+05 cfu/ml. In all treatments, it was shown that the relative values of the central point of the Vibrio parahaemolyticus colonies remained within the mean of 9.80E+04 cfu/ml (see, FIG. 10).

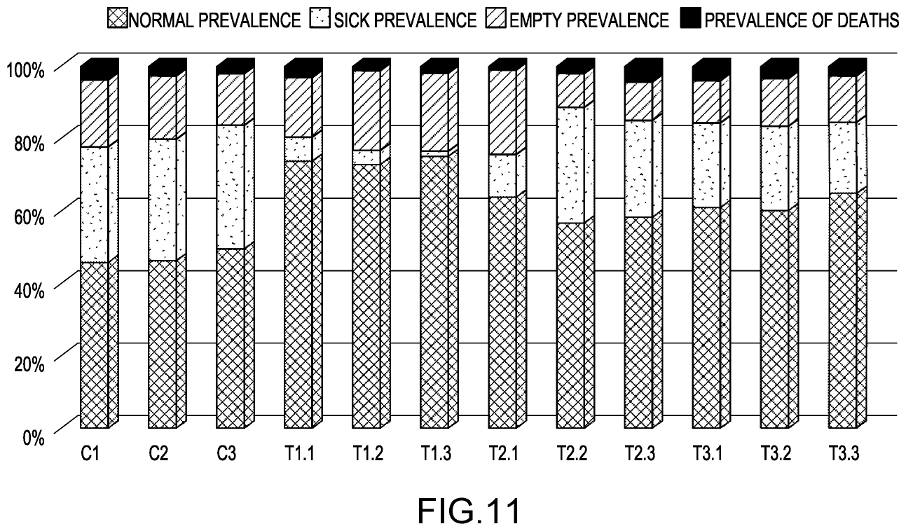

At the beginning of the study, it was observed that the control began with a prevalence 22.64% of cases of Vibrio. 14.83% of shrimp who did not feed properly had empty intestines and a mortality of 3.21%.
T1 treatments: (5.24% of the shrimp) 19.22% had empty intestines and there was a 2.00% mortality rate (see, FIG. 11).
T2 treatments: (29.26% of the shrimp) 9.82% had empty intestines and there was a 0.5% mortality rate (see, FIG. 11).
T3 treatments: (23.34% of the shrimp) 12.41% had empty intestines and there was a 2.65% mortality rate (see, FIG. 11).
Control: (32.70% of the shrimp) 17.87% had empty intestines and there was 23.85% mortality rate (see, FIG. 11).

The pre-juvenile shrimp fed T2 presented at 0.047 g after seven (7) days with respect to the control with 0.0215 g. This indicated that the animals had a better feed conversion of 1.98 compared to the feed conversion of the control of 3.96. Thus, it can be concluded that T2 is the pre-juvenile stage that is the best treatment in Litopenaeus vannamei. The shrimp feeding on T1, T2, and T3 all demonstrated enhanced immunity against Vibrio during all the days of testing the animals exposed to Vibrio. At the end of the study, the survival rate of T2 feed animals was 99.5%, while the control had a survival rate of 76.15%.

It was determined that T2 demonstrated the most significant effectiveness, reaching a mean of 99.5% survival and a final average weight of 0.043 g with respect to the control. T1 and T3 demonstrated only a slightly lower effectiveness than T2. The average survival of the subjects receiving treatments T1, T2, and T3 was 98.28% (see Table 2). T2 was most efficacious in providing shrimp with enhanced immunity against Vibrio.

### Example 6. Materials and Methods for Expressing Vectors in Diatom Cells

The plasmid vectors shown in FIGS. 1A and 1B were cloned as described in Example 1. Shbel gene was used a selectable marker, which provides resistance against the antibiotic Zeocin. The vectors were co-transformed into diatom cells. Nitrate reductase promoters and terminators from the Thalassiosira pseudonana genome were used as control elements.

### Example 7. Immunization of Tilapia

Tilapia fish were subjected to the following immunization regimens: (i) oral immunization through once-weekly feeding; (ii) oral immunization through twice-weekly feeding; and (iii) immunization via intraperitoneal injection.

### Materials and Methods

Cell Cultures and Antigen Expression: Diatom cells were utilized as hosts for the expression of the antigen (pVX_192), which was tagged with enhanced Green Fluorescent Protein (eGFP). The diatom cells were subjected to either intraperitoneal immunization (PI) through injection or oral administration to five tilapia individuals per treatment group.

Immunization Regimen: The injection group received an initial immunization and was subsequently boosted in the third week using the antigen (pVX_192) formulation. Meanwhile, two feeding groups were established: Group A and Group D were fed with standard commercial fish feed, while Group B and Group C were fed fish diet composed of 10% diatom biomass expressing the antigen (pVX_192) once a week and twice a week, respectively.

Blood Sampling and Pooling: At the culmination of the fourth week, blood samples were collected from each fish and pooled to create representative serum samples for analysis.

Antibody Detection using ELISA: Antibody generation against the eGFP tag was assessed using the enzyme-linked immunosorbent assay (ELISA) technique. ELISA plates were coated with eGFP (5 μg) and allowed to incubate overnight. Subsequently, the plates were incubated with serum samples (diluted 1:40) derived from the experimental groups. Following this, anti-mouse IgG monoclonal antibody (dilution 1/33, Aquatic Diagnostics, UK) was applied, followed by an anti-mouse secondary antibody that was conjugated to horseradish peroxidase (HRP). The development of color was achieved using a substrate solution (Cell Biolabs), and the enzymatic reaction was halted using a stop solution. Quantification of the ELISA signal was conducted using an ELISA reader (Molecular Devices) at an absorbance of A450.

### Results-Antibody Generation in Immunized Fish

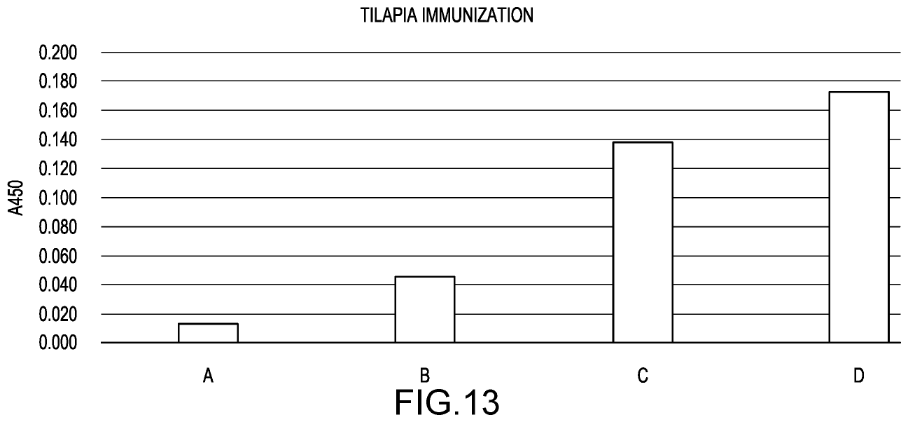

Absorbance results from the ELISA assay are shown in FIG. 13. Group A. shows data from a non-immunizing group of Tilapia fish that functioned as a control. Group B. shows Tilapia fish that were treated with oral immunization through once-weekly feeding. Group C. shows Tilapia fish that were treated with oral immunization through twice-weekly feeding. Group D. shows Tilapia fish that were treated through intraperitoneal injection.

Immunization Route Influence on Antibody Production: Both injection and oral administration of the antigen (pVX_192) resulted in the generation of antibodies targeting the eGFP tag. Notably, fish subjected to injection exhibited the most substantial antibody production, characterized by the highest antibody titers.

Impact of Oral Administration on Antibody Response: Fish administered with a diet comprising 10% diatom biomass displayed the ability to produce antibodies against the eGFP tag. This finding suggests that the oral delivery of the antigen-expressing diatoms effectively triggered an immune response.

Frequency-Dependent Antibody Production: The frequency of administration through oral intake played a role in the magnitude of antibody generation. Fish receiving the antigen-expressing diatoms twice a week demonstrated elevated levels of antibody production compared to those fed once a week. This observation underscores the relationship between the frequency of exposure and the immune response potency in generating antibodies against the vaccine candidates.

### Additional Comments

While various illustrative examples are described above, it will be apparent to one skilled in the art that various changes and modifications may be made therein without departing from the disclosure. The appended claims are intended to cover all such changes and modifications that fall within the true spirit and scope of the examples provided herein.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

Sequences: The following sequences are referred to herein:

| SEQ | | |
|---|---|---|
| ID | | |
| NO: | Description | Sequence |
| 1 | Nitrate Reductase | Agagggcaattgattgcggtatatgaaacgtgcttcttgttgctgtaaaaataactcgtcgaacaagtcat |
| | Promoter | ccgactagcgcctctggtgatcaaaccacgcacgtacggattggtttgatggttatcctcggattggtatc |
| | | cttagaacatcttcgaaggttctaggagactacccgcaaaacggatggtatttccccccaacatccaaaat |
| | | tggaatgccacgtcatctgcacttccggactctggcagcagcaaaatggcgcgctcgaccacatcacg |
| | | gttctccaccctcttccgacagcaccaacatctctttccggaagaaccaagctggaggatgaagatgacg |
| | | atgagaagaggtgtggacggggtgaaaagaggctcgggaaggcttttcggcaagcacagagcttttgc |
| | | acataacaacctctcacaattcaccaacccatccaattgacagcctccatcttttaacatttcaacacaaag |
| | | gaaccaaca |
| 2 | Nitrate Reductase | Tgaggatgctcatcgttttgactttgttggatgacgcctcttgtacatactgattagacatgggagtgttggt |
| | | tgaattgtagatagtttacaccggctgttaactgaagagatactattacactattcatcatcattccaattga |
| | | tacgtcgccgccacgcactgctcatccatagaaccctcagacacacttcacctcaaaggttcgtatctccatc |
| | | ggctccaaagaaaccacactgttgccttgctccagcttacgactatcctccttgaaatcatcatcgttccac |
| | | cactgaaac |
| 3 | Terminator | ATGGCACAAGTCATCAACACCAACTCTCTCTCTCTCTTGACCC |
| | Nucleotide sequence | AGAACAACCTCAACAAGTCCCAGTCCGCTCTCGGAACCGCTAT |
| | of Flagellin (codon- | CGAGCGTCTCTCTTCCGGACTCCGTATCAACTCTGCTAAGGAC |
| | optimized) | GATGCTGCAGGACAGGCTATCGCTAACCGTTTCACCGCTAACA |
| | | TCAAGGGACTCACTCAGGCTTCCCGTAACGCTAACGACGGAAT |
| | | CTCCATCGCTCAGACCACTGAGGGAGCTCTCAACGAGATCAAC |
| | | AACAACCTCCAGCGTGTGCGTGAGCTCGCTGTTCAGTCTGCTA |
| | | ACTCTACCAACTCCCAGTCTGACCTCGACTCCATCCAGGCTGA |
| | | GATCACCCAGCGTCTCAACGAGATCGACCGTGTGTCCGGACAG |
| | | ACTCAGTTCAACGGAGTGAAGGTCCTCGCTCAGGACAACACCC |
| | | TCACCATCCAGGTTGGAGCCAACGACGGAGAGACTATCGATAT |
| | | CGATCTCAAGCAGATCAACTCTCAGACCCTCGGACTCGATACC |
| | | CTCAACGTGCAACAAAAGTACAAGGTCTCTGATACCGCTGCAA |
| | | CTGTTACCGGATACGCCGATACTACCATCGCTCTCGACAACAG |
| | | TACTTTCAAGGCCTCTGCTACTGGACTCGGAGGAACTGACCAG |
| | | AAGATCGATGGAGATCTCAAGTTCGATGATACCACTGGAAAGT |
| | | ACTACGCCAAGGTTACCGTTACCGGAGGAACTGGAAAGGATG |
| | | GATACTACGAGGTTTCCGTTGATAAGACCAACGGAAAGGTGAC |
| | | TCTCGCTGGAGGAGCTACTTCCCCACTCACCGGAGGACTCCCT |
| | | GCTACCGCAACTGAGGATGTGAAGAACGTGCAAGTTGCAAAC |
| | | GCTGATTTGACCGAGGCTAAGGCCGCATTGACCGCAGCAGGA |
| | | GTTACCGGAACCGCATCTGTTGTTAAGATGTCTTACACTGATA |
| | | ACAACGGAAAGACTATCGATGGAGGACTCGCAGTTAAGGTGG |
| | | GAGATGATTACTACTCTGCAACTCAAAACAAGGATGGATCCAT |
| | | CAGTATCAACACTACCAAGTACACTGCAGATGACGGAACCTCC |
| | | AAGACTGCACTCAACAAGCTCGGAGGAGCAGACGGAAAGACC |
| | | GAGGTTGTTTCTATCGGAGGAAAGACTTACGCTGCAAGTAAGG |
| | | CCGAGGGACACAACTTCAAGGCACAGCCTGATCTCGCTGAGG |
| | | CTGCTGCTACCACCACCGAGAACCCACTCCAGAAGATCGATGC |
| | | TGCTTTGGCACAGGTTGACACCCTCCGTTCTGACCTCGGAGCT |
| | | GTGCAGAACCGTTTCAACTCCGCTATCACCAACCTCGGAAACA |
| | | CCGTGAACAACCTCACTTCTGCCCGTTCTCGTATCGAGGATTCC |
| | | GACTACGCTACCGAGGTTTCCAACATGTCTCGTGCTCAGATCC |
| | | TCCAGCAGGCCGGAACCTCCGTTCTCGCTCAGGCTAACCAGGT |
| | | TCCACAAAACGTCCTCTCTCTCCTCCGT |
| 4 | Amino acid | MAQVINTNSLSLLTQNNLNKSQSALGTAIERLSSGLRINSAKDDA |
| | sequence of | AGQAIANRFTANIKGLTQASRNANDGISIAQTTEGALNEINNNLQR |
| | Flagellin | VRELAVQSANSTNSQSDLDSIQAEITQRLNEIDRVSGQTQFNGVK |
| | | VLAQDNTLTIQVGANDGETIDIDLKQINSQTLGLDTLNVQQKYKV |
| | | SDTAATVTGYADTTIALDNSTFKASATGLGGTDQKIDGDLKFDDT |
| | | TGKYYAKVTVTGGTGKDGYYEVSVDKTNGKVTLAGGATSPLTG |
| | | GLPATATEDVKNVQVANADLTEAKAALTAAGVTGTASVVKMSY |
| | | TDNNGKTIDGGLAVKVGDDYYSATQNKDGSISINTTKYTADDGT |
| | | SKTALNKLGGADGKTEVVSIGGKTYAASKAEGHNFKAQPDLAEA |
| | | AATTTENPLQKIDAALAQVDTLRSDLGAVQNRFNSAITNLGNTVN |
| | | NLTSARSRIEDSDYATEVSNMSRAQILQQAGTSVLAQANQVPQN |
| | | VLSLLR |
| 5 | Gene cloned [Vibrio | ATGTGTTCTTCCTTGCCAGAGGAGTTGAACGCAAGTACCGAGC |
| | parahaemolyticus | AAGTGGTTACTGATTACAAGGCATTCGCTGAGTCTCAGGGAGA |
| | OMP (codon- | GTTGACCAACGACGTTCGTCTCGGAGGAATCATCGCAAAGGTC |
| | optimized)] | GATAACTTCAAGGACAAGACTCGTTTGGAGATCGTGAACCTCC |
| | | CAATCAACAAGTCTGGAAAGCCAGACATCGACCAAGAGCCTA |
| | | CCGGACGTTTCGCTGTGTACTTCGATGGATACTTGGAGCCTGTT |
| | | GCCTTCTCCCAGGGACGTCTCGTTACTATCGTCGGAAAGGGAG |
| | | CCGGAGAGGAGGAGGGAAAGATCGGAGAGCACGAGTACGTGT |
| | | TCCCATTGGTTAAGGGACAAGGATACCGTCTCTGGAAGATCGA |
| | | GGAGCGTGTGCGTATGTACGACAGTCCAACCTACTTCTACCCT |
| | | TGTTACTCTATCAACTGCCGTATGCTCCGTAACGATTTCCCTCA |
| | | AGACGGAAAGGTCATCAAGCAGGTGAAG |
| 6 | Vibrio | MCSSLPEELNASTEQVVTDYKAFAESQGELTNDVRLGGIIAKVDN |
| | parahaemolyticus | FKDKTRLEIVNLPINKSGKPDIDQEPTGRFAVYFDGYLEPVAFSQG |
| | Slp family | RLVTIVGKGAGEEEGKIGEHEYVFPLVKGQGYRLWKIEERVRMY |
| | lipoprotein | DSPTYFYPCYSINCRMLRNDFPQDGKVIKQVK |
| 7 | eGFP tag | atggtgagcaagggcgaggagctgttcaccggggtggtgcccatcctggtcgagctggacggcgacg |
| | | taaacggccacaagttcagcgtgtccggcgagggcgagggcgatgccacctacggcaagctgaccct |
| | | gaagttcatctgcaccaccggcaagctgcccgtgccctggcccaccctcgtgaccaccctgacctacg |
| | | gcgtgcagtgcttcagccgctaccccgaccacatgaagcagcacgacttcttcaagtccgccatgcccg |
| | | aaggctacgtccaggagcgcaccatcttcttcaaggacgacggcaactacaagacccgcgccgaggt |
| | | gaagttcgagggcgacaccctggtgaaccgcatcgagctgaagggcatcgacttcaaggaggacggc |
| | | aacatcctggggcacaagctggagtacaactacaacagccacaacgtctatatcatggccgacaagca |
| | | gaagaacggcatcaaggtgaacttcaagatccgccacaacatcgaggacggcagcgtgcagctcgcc |
| | | gaccactaccagcagaacacccccatcggcgacggccccgtgctgctgcccgacaaccactacctga |
| | | gcacccagtccgccctgagcaaagaccccaacgagaagcgcgatcacatggtcctgctggagttcgtg |
| | | accgccgccgggatcactctcggcatggacgagctgtacaagtaa |
| 8 | eGFP | MVSKGEELFTGVVPILVELDGDVNGHKFSVSGEGEGDATYGKLT |
| | | LKFICTTGKLPVPWPTLVTTLTYGVQCFSRYPDHMKQHDFFKSA |
| | | MPEGYVQERTIFFKDDGNYKTRAEVKFEGDTLVNRIELKGIDFKE |
| | | DGNILGHKLEYNYNSHNVYIMADKQKNGIKVNFKIRHNIEDGSV |
| | | QLADHYQQNTPIGDGPVLLPDNHYLSTQSALSKDPNEKRDHMVL |
| | | LEFVTAAGITLGMDELYK- |
| 9 | Sh ble (codon- | atggctaagttgacttccgccgttcccgttttgaccgctcgtgatgtggcaggagcagttgagttttggacc |
| | optimized)- | gaccgtcttggattcagtcgtgatttcgttgaggatgactttgcaggagtggttagggatgacgtcacgctt |
| | selectable marker | ttcatctcggcagtgcaagatcaggtcgtaccagacaacaccctcgcttgggtctgggttcgtggactcg |
| | | atgagttgtacgccgagtggtctgaagtggtttccaccaattttcgtgacgcttctggaccagccatgactg |
| | | agattggagaacaaccttggggaagggaatttgccttgagggaccccgcaggaaactgtgttcactttgt |
| | | ggctgaggagcaggattga |
| 10 | Sh ble | MAKLTSAVPVLTARDVAGAVEFWTDRLGFSRDFVEDDFAGVVR |
| | | DDVTLFISAVQDQVVPDNTLAWVWVRGLDELYAEWSEVVSTNF |
| | | RDASGPAMTEIGEQPWGREFALRDPAGNCVHFVAEEQD- |
| 11 | Italics: FCP | gggggatccactagttctagagcggccggccgccaccgcggtggagctccagcttttgttccctttagtg |
| | promoter and | agggttaattgcgcgcttggcgtaatcatggtcatagctgtttcctgtgtgaaattgttatccgctcacaatt |
| | terminator | ccacacaacatacgagccggaagcataaagtgtaaagcctggggtgcctaatgagtgagctaactcacat |
| | Bold: Gene (Sh ble) | taattgcgttgcgctcactgcccgctttccagtcgggaaacctgtcgtgccagctgcattaatgaatcggc |
| | | caacgcgcggggagaggcggtttgcgtattgggcgctcttccgcttcctcgctcactgactcgctgcgct |
| | | cggtcgttcggctgcggcgagcggtatcagctcactcaaaggcggtaatacggttatccacagaatcag |
| | | gggataacgcaggaaagaacatgtgagcaaaaggccagcaaaaggccaggaaccgtaaaaaggcc |
| | | gcgttgctggcgtttttccataggctccgcccccctgacgagcatcacaaaaatcgacgctcaagtcaga |
| | | ggtggcgaaacccgacaggactataaagataccaggcgtttccccctggaagctccctcgtgcgctctc |
| | | ctgttccgaccctgccgcttaccggatacctgtccgcctttctcccttcgggaagcgtggcgctttctcata |
| | | gctcacgctgtaggtatctcagttcggtgtaggtcgttcgctccaagctgggctgtgtgcacgaaccccc |
| | | cgttcagcccgaccgctgcgccttatccggtaactatcgtcttgagtccaacccggtaagacacgacttat |
| | | cgccactggcagcagccactggtaacaggattagcagagcgaggtatgtaggcggtgctacagagttc |
| | | ttgaagtggtggcctaactacggctacactagaaggacagtatttggtatctgcgctctgctgaagccagt |
| | | taccttcggaaaaagagttggtagctcttgatccggcaaacaaaccaccgctggtagcggtggtttttttgt |
| | | ttgcaagcagcagattacgcgcagaaaaaaaggatctcaagaagatcctttgatcttttctacggggtctg |
| | | acgctcagtggaacgaaaactcacgttaagggattttggtcatgagattatcaaaaaggatcttcacctag |
| | | atccttttaaattaaaaatgaagttttaaatcaatctaaagtatatatgagtaaacttggtctgacagttacc |
| | | aatgcttaatcagtgaggcacctatctcagcgatctgtctatttcgttcatccatagttgcctgactccccgt |
| | | cgtgtagataactacgatacgggagggcttaccatctggccccagtgctgcaatgataccgcgagacccac |
| | | gctcaccggctccagatttatcagcaataaaccagccagccggaagggccgagcgcagaagtggtcct |
| | | gcaactttatccgcctccatccagtctattaattgttgccgggaagctagagtaagtagttcgccagttaata |
| | | gtttgcgcaacgttgttgccattgctacaggcatcgtggtgtcacgctcgtcgtttggtatggcttcattcag |
| | | ctccggttcccaacgatcaaggcgagttacatgatcccccatgttgtgcaaaaaagcggttagctccttcg |
| | | gtcctccgatcgttgtcagaagtaagttggccgcagtgttatcactcatggttatggcagcactgcataatt |
| | | ctcttactgtcatgccatccgtaagatgcttttctgtgactggtgagtactcaaccaagtcattctgagaata |
| | | gtgtatgcggcgaccgagttgctcttgcccggcgtcaatacgggataataccgcgccacatagcagaac |
| | | tttaaaagtgctcatcattggaaaacgttcttcggggcgaaaactctcaaggatcttaccgctgttgagatc |
| | | cagttcgatgtaacccactcgtgcacccaactgatcttcagcatcttttactttcaccagcgtttctgggtga |
| | | gcaaaaacaggaaggcaaaatgccgcaaaaaagggaataagggcgacacggaaatgttgaatactca |
| | | tactcttcctttttcaatattattgaagcatttatcagggttattgtctcatgagcggatacatatttgaatg |
| | | tatttagaaaaataaacaaataggggttccgcgcacatttccccgaaaagtgccacctaaattgtaagcgtta |
| | | atattttgttaaaattcgcgttaaatttttgttaaatcagctcattttttaaccaataggccgaaatcggcaa |
| | | aatcccttataaatcaaaagaatagaccgagatagggttgagtgttgttccagtttggaacaagagtccacta |
| | | ttaaagaacgtggactccaacgtcaaagggcgaaaaaccgtctatcagggcgatggcccactacgtgaac |
| | | catcaccctaatcaagttttttggggtcgaggtgccgtaaagcactaaatcggaaccctaaagggagccc |
| | | ccgatttagagcttgacggggaaagccggcgaacgtggcgagaaaggaagggaagaaagcgaaag |
| | | gagcgggcgctagggcgctggcaagtgtagcggtcacgctgcgcgtaaccaccacacccgccgcgc |
| | | ttaatgcgccgctacagggcgcgtcccattcgccattcaggctgcgcaactgttgggaagggcgatcgg |
| | | tgcgggcctcttcgctattacgccagctggcgaaagggggatgtgctgcaaggcgattaagttgggtaa |
| | | cgccagggttttcccagtcacgacgttgtaaaacgacggccagtgagcgcgcgtaatacgactcactat |
| | | agggcgaattgggtacgtaccgggccccccctcgaggtcgacggtatcgataagcttgcgctttttccg |
| | | agaactccccataagtcaacggctccaatcaagaatgtatccgacaacggcgagcatagcaacac |
| | | gtccgtctttggagtagaatcatcatgttgtggatgaatacacagatgaatgacattaaaagcatgaa |
| | | catgttagagagtaggaggtagagattgatatggtagcattgcgatgtttgtttttggtcagcatatgat |
| | | gagtggataccaatatgatgaaagttgaatctcgcgtttgagctcagcggtacgttattgatcgaaagt |
| | | agcctgatcaaaatccttggagagtacaagaggatcaaagaatccagtgggggcgataactccaa |
| | | gctcgttctcaaagaggcaatggaggtagaaactcatcccagttgagaagaagtgaaggcagtgg |
| | | cggtggcgaaagcagaggcaacgaggacagacttcctgtgggttgatgcaacgaatatttccagaa |
| | | ggagaagtttagagagttgaaccgctacctacaatgacaaagtatcgtatcgattttgatgttggttggt |
| | | tatgaattcaaactgtaagttggattgtgagaagatcagaagttgaacgaacacatctttccgatcatt |
| | | cacctccacactgcaacaacacggtacttcttccgcggcaggtctctgtcgccattctcttgtcctgttgt |
| | | tggctgtgagacgaggaaagcaacgacaagtttcacadaagggagttcctttaacgagatatgttttt |
| | | tataaagagtcccaatagaaagacaaattgattcctccgtgcaaacgcgcaaataaacaccacgtc |
| | | cattatatccatatctttcagagtatccaacaagtgttgaaggacaggtagttgaagtaacgtatcttcc |
| | | ccctcgactggatccatcaacaaggcgaacaaatccattcaacctctcataaattatctgatttaccaa |
| | | accATGGCTAAGTTGACTTCCGCCGTTCCCGTTTTGACCGCT |
| | | CGTGATGTGGCAGGAGCAGTTGAGTTTTGGACCGACCGTC |
| | | TTGGATTCAGTCGTGATTTCGTTGAGGATGACTTTGCAGGA |
| | | GTGGTTAGGGATGACGTCACGCTTTTCATCTCGGCAGTGC |
| | | AAGATCAGGTCGTACCAGACAACACCCTCGCTTGGGTCTG |
| | | GGTTCGTGGACTCGATGAGTTGTACGCCGAGTGGTCTGAA |
| | | GTGGTTTCCACCAATTTTCGTGACGCTTCTGGACCAGCCAT |
| | | GACTGAGATTGGAGAACAACCTTGGGGAAGGGAATTTGCC |
| | | TTGAGGGACCCCGCAGGAAACTGTGTTCACTTTGTGGCTG |
| | | AGGAGCAGGATTGAatactggattggtgaatcaatgagccgtagcacaatggttacatt |
| | | cggctagctaagatccaatggcaaggaccaagtgctggaacttgttttgctttagcagatcttagcgtg |
| | | agaggtatttgtcctctgtcaggagtagatagtagatgttctttttaaactaaaatgctaactgttccgaa |
| | | ttcctcatcgcagctaatccgtacatcaaaagacaaaatgctaggtatgtgtactacatctcctgttgct |
| | | agataagacatatgataggaaacacaccatcaatagtcattgtagctttacttatactacgcatttgca |
| | | ctttcccctgagtggcagaggcgcattgagaaaatcgatctcaacatagtttatgtagcatcccctaga |
| | | tccattactttaagtctccttcgtctttggtgtaggcatgttggacacaacgaggtaaaacacaacaca |
| | | aacaatgtgtccagcaaagtagtagctgctccagttctccc |
| 12 | Flagellin + Vibrio | cgggggatccactagttctagagcggccggccgccaccgcggtggagctccagcttttgttccctttagt |
| | parahaemolyticus | gagggttaattgcgcgcttggcgtaatcatggtcatagctgtttcctgtgtgaaattgttatccgctcacaat |
| | Slp/YeaY family | tccacacaacatacgagccggaagcataaagtgtaaagcctggggtgcctaatgagtgagctaactcac |
| | lipoprotein OMP | attaattgcgttgcgctcactgcccgctttccagtcgggaaacctgtcgtgccagctgcattaatgaatcg |
| | Italics: NR promoter | gccaacgcgcggggagaggcggtttgcgtattgggcgctcttccgcttcctcgctcactgactcgctgc |
| | and terminator | gctcggtcgttcggctgcggcgagcggtatcagctcactcaaaggcggtaatacggttatccacagaat |
| | CAPITAL | caggggataacgcaggaaagaacatgtgagcaaaaggccagcaaaaggccaggaaccgtaaaaag |
| | UNDERLINED | gccgcgttgctggcgtttttccataggctccgcccccctgacgagcatcacaaaaatcgacgctcaagtc |
| | Flagellin | agaggtggcgaaacccgacaggactataaagataccaggcgtttccccctggaagctccctcgtgcgc |
| | Bold = OMP | tctcctgttccgaccctgccgcttaccggatacctgtccgcctttctcccttcgggaagcgtggcgctttct |
| | Lower underlined = | catagctcacgctgtaggtatctcagttcggtgtaggtcgttcgctccaagctgggctgtgtgcacgaacc |
| | eGFP | ccccgttcagcccgaccgctgcgccttatccggtaactatcgtcttgagtccaacccggtaagacacgac |
| | | ttatcgccactggcagcagccactggtaacaggattagcagagcgaggtatgtaggcggtgctacaga |
| | | gttcttgaagtggtggcctaactacggctacactagaaggacagtatttggtatctgcgctctgctgaagc |
| | | cagttaccttcggaaaaagagttggtagctcttgatccggcaaacaaaccaccgctggtagcggtggtttt |
| | | tttgtttgcaagcagcagattacgcgcagaaaaaaaggatctcaagaagatcctttgatcttttctacgggg |
| | | tctgacgctcagtggaacgaaaactcacgttaagggattttggtcatgagattatcaaaaaggatcttcac |
| | | ctagatccttttaaattaaaaatgaagttttaaatcaatctaaagtatatatgagtaaacttggtctgacagt |
| | | taccaatgcttaatcagtgaggcacctatctcagcgatctgtctatttcgttcatccatagttgcctgactcc |
| | | ccgtcgtgtagataactacgatacgggagggcttaccatctggccccagtgctgcaatgataccgcgagac |
| | | ccacgctcaccggctccagatttatcagcaataaaccagccagccggaagggccgagcgcagaagtg |
| | | gtcctgcaactttatccgcctccatccagtctattaattgttgccgggaagctagagtaagtagttcgccag |
| | | ttaatagtttgcgcaacgttgttgccattgctacaggcatcgtggtgtcacgctcgtcgtttggtatggcttc |
| | | attcagctccggttcccaacgatcaaggcgagttacatgatcccccatgttgtgcaaaaaagcggttagct |
| | | ccttcggtcctccgatcgttgtcagaagtaagttggccgcagtgttatcactcatggttatggcagcactgc |
| | | ataattctcttactgtcatgccatccgtaagatgcttttctgtgactggtgagtactcaaccaagtcattctg |
| | | agaatagtgtatgcggcgaccgagttgctcttgcccggcgtcaatacgggataataccgcgccacatagc |
| | | agaactttaaaagtgctcatcattggaaaacgttcttcggggcgaaaactctcaaggatcttaccgctgttg |
| | | agatccagttcgatgtaacccactcgtgcacccaactgatcttcagcatcttttactttcaccagcgtttctg |
| | | ggtgagcaaaaacaggaaggcaaaatgccgcaaaaaagggaataagggcgacacggaaatgttgaa |
| | | tactcatactcttcctttttcaatattattgaagcatttatcagggttattgtctcatgagcggatacatatt |
| | | tgaatgtatttagaaaaataaacaaataggggttccgcgcacatttccccgaaaagtgccacctaaattgtaa |
| | | gcgttaatattttgttaaaattcgcgttaaatttttgttaaatcagctcattttttaaccaataggccgaaat |
| | | cggcaaaatcccttataaatcaaaagaatagaccgagatagggttgagtgttgttccagtttggaacaagagt |
| | | ccactattaaagaacgtggactccaacgtcaaagggcgaaaaaccgtctatcagggcgatggcccactacgt |
| | | gaaccatcaccctaatcaagttttttggggtcgaggtgccgtaaagcactaaatcggaaccctaaaggga |
| | | gcccccgatttagagcttgacggggaaagccggcgaacgtggcgagaaaggaagggaagaaagcg |
| | | aaaggagcgggcgctagggcgctggcaagtgtagcggtcacgctgcgcgtaaccaccacacccgcc |
| | | gcgcttaatgcgccgctacagggcgcgtcccattcgccattcaggctgcgcaactgttgggaagggcg |
| | | atcggtgcgggcctcttcgctattacgccagctggcgaaagggggatgtgctgcaaggcgattaagttg |
| | | ggtaacgccagggttttcccagtcacgacgttgtaaaacgacggccagtgagcgcgcgtaatacgactc |
| | | actatagggcgaattgggtaccgggcccccccagagggcaattgattgcggtatatgaaacgtgcttc |
| | | ttgttgctgtaaaaataactcgtcgaacaagtcatccgactagcgcctctggtgatcaaaccacgcac |
| | | gtacggattggtttgatggttatcctcggattggtatccttagaacatcttcgaaggttctaggagactac |
| | | ccgcaaaacggatggtatttccccccaacatccaaaattggaatgccacgtcatctgcacttccggac |
| | | tctggcagcagcaaaatggcgcgctcgaccacatcacggttctccaccctcttccgacagcaccaac |
| | | atctctttccggaagaaccaagctggaggatgaagatgacgatgagaagaggtgtggacggggtg |
| | | aaaagaggctcgggaaggcttttcggcaagcacagagcttttgcacataacaacctctcacaattca |
| | | ccaacccatccaattgacagcctccatcttttaacatttcaacacaaaggaaccaacagatatcataa |
| | | tcATGGCACAAGTCATCAACACCAACTCTCTCTCTCTCTTGACC |
| | | CAGAACAACCTCAACAAGTCCCAGTCCGCTCTCGGAACCGCTA |
| | | TCGAGCGTCTCTCTTCCGGACTCCGTATCAACTCTGCTAAGGA |
| | | CGATGCTGCAGGACAGGCTATCGCTAACCGTTTCACCGCTAAC |
| | | ATCAAGGGACTCACTCAGGCTTCCCGTAACGCTAACGACGGAA |
| | | TCTCCATCGCTCAGACCACTGAGGGAGCTCTCAACGAGATCAA |
| | | CAACAACCTCCAGCGTGTGCGTGAGCTCGCTGTTCAGTCTGCT |
| | | AACTCTACCAACTCCCAGTCTGACCTCGACTCCATCCAGGCTG |
| | | AGATCACCCAGCGTCTCAACGAGATCGACCGTGTGTCCGGACA |
| | | GACTCAGTTCAACGGAGTGAAGGTCCTCGCTCAGGACAACACC |
| | | CTCACCATCCAGGTTGGAGCCAACGACGGAGAGACTATCGAT |
| | | ATCGATCTCAAGCAGATCAACTCTCAGACCCTCGGACTCGATA |
| | | CCCTCAACGTGCAACAAAAGTACAAGGTCTCTGATACCGCTGC |
| | | AACTGTTACCGGATACGCCGATACTACCATCGCTCTCGACAAC |
| | | AGTACTTTCAAGGCCTCTGCTACTGGACTCGGAGGAACTGACC |
| | | AGAAGATCGATGGAGATCTCAAGTTCGATGATACCACTGGAA |
| | | AGTACTACGCCAAGGTTACCGTTACCGGAGGAACTGGAAAGG |
| | | ATGGATACTACGAGGTTTCCGTTGATAAGACCAACGGAAAGGT |
| | | GACTCTCGCTGGAGGAGCTACTTCCCCACTCACCGGAGGACTC |
| | | CCTGCTACCGCAACTGAGGATGTGAAGAACGTGCAAGTTGCA |
| | | AACGCTGATTTGACCGAGGCTAAGGCCGCATTGACCGCAGCA |
| | | GGAGTTACCGGAACCGCATCTGTTGTTAAGATGTCTTACACTG |
| | | ATAACAACGGAAAGACTATCGATGGAGGACTCGCAGTTAAGG |
| | | TGGGAGATGATTACTACTCTGCAACTCAAAACAAGGATGGATC |
| | | CATCAGTATCAACACTACCAAGTACACTGCAGATGACGGAACC |
| | | TCCAAGACTGCACTCAACAAGCTCGGAGGAGCAGACGGAAAG |
| | | ACCGAGGTTGTTTCTATCGGAGGAAAGACTTACGCTGCAAGTA |
| | | AGGCCGAGGGACACAACTTCAAGGCACAGCCTGATCTCGCTG |
| | | AGGCTGCTGCTACCACCACCGAGAACCCACTCCAGAAGATCG |
| | | ATGCTGCTTTGGCACAGGTTGACACCCTCCGTTCTGACCTCGG |
| | | AGCTGTGCAGAACCGTTTCAACTCCGCTATCACCAACCTCGGA |
| | | AACACCGTGAACAACCTCACTTCTGCCCGTTCTCGTATCGAGG |
| | | ATTCCGACTACGCTACCGAGGTTTCCAACATGTCTCGTGCTCA |
| | | GATCCTCCAGCAGGCCGGAACCTCCGTTCTCGCTCAGGCTAAC |
| | | CAGGTTCCACAAAACGTCCTCTCTCTCCTCCGTATGTGTTCTT |
| | | CCTTGCCAGAGGAGTTGAACGCAAGTACCGAGCAAGTGGT |
| | | TACTGATTACAAGGCATTCGCTGAGTCTCAGGGAGAGTTG |
| | | ACCAACGACGTTCGTCTCGGAGGAATCATCGCAAAGGTCG |
| | | ATAACTTCAAGGACAAGACTCGTTTGGAGATCGTGAACCTC |
| | | CCAATCAACAAGTCTGGAAAGCCAGACATCGACCAAGAGC |
| | | CTACCGGACGTTTCGCTGTGTACTTCGATGGATACTTGGAG |
| | | CCTGTTGCCTTCTCCCAGGGACGTCTCGTTACTATCGTCGG |
| | | AAAGGGAGCCGGAGAGGAGGAGGGAAAGATCGGAGAGCA |
| | | CGAGTACGTGTTCCCATTGGTTAAGGGACAAGGATACCGT |
| | | CTCTGGAAGATCGAGGAGCGTGTGCGTATGTACGACAGTC |
| | | CAACCTACTTCTACCCTTGTTACTCTATCAACTGCCGTATG |
| | | CTCCGTAACGATTTCCCTCAAGACGGAAAGGTCATCAAGC |
| | | AGGTGAAGatggtgagcaagggcgaggagctgttcaccggggtggtgcccatcctggtcgag |
| | | ctggacggcgacgtaaacggccacaagttcaggtgtccggcgagggcgaggggatgccacctac |
| | | ggcaagctgaccctgaagttcatctgacgcaagctgcccccctggcccaccctcgtgac |
| | | caccctgacctacggcgtgcagtgcttcagccgctaccccgaccacatgaagcagcacgacttcttcaa |
| | | gtccgccatgcccgaaggctacgtccaggagcgcaccatcttcttcaaggacgacggcaactacaaga |
| | | cccgcgccgaggtgaagttcgagggcgacaccctggtgaaccgcatcgagctgaagggcatcgactt |
| | | caaggaggacggcaacatcctggggcacaagctggagtacaactacaacagccacaacgtctatatca |
| | | tggccgacaagcagaagaacggcatcaaggtgaacttcaagatccgccacaacatcgaggacggca |
| | | gcgtgcagctcgccgaccactaccagcagaacacccccatcggcgacggccccgtgctgctgcccga |
| | | caaccactacctgagcacccagtccgccctgagcaaagaccccaacgagaagcgcgatcacatggtc |
| | | ctgctggagttcgtgaccgccgccgggatcactctcggcatggacgagctgtacaagtaagcggccgc |
| | | tgaggatgctcatcgttttgactttgttggatgacgcctcttgtacatactgattagacatgggagtgttgg |
| | | ttgaattgtagatagtttacaccggctgttaactgaagagatactattacactattcatcatcattccaatt |
| | | gatacgtcgccgccacgcactgctcatccatagaaccctcagacacacttcacctcaaaggttcgtat |
| | | ctccatcggctccaaagaaaccacactgttgccttgctccagcttacgactatcctccttgaaatcatca |
| | | tcgttccaccactgaaactgtctctgttcccactcatctcttctctgattaccagagagtgtcgtttccgtg |
| | | atggagactactttgcgagaaggaaacaagacgccaagatccacctcaaccggaagggagtattg |
| | | atgactttctcccactccgtactgatgcccaagcctgacaagataggtgtcgtcttgctctagtgatgca |
| | | aacgttaccagcatgatattatcaggcaatgcatcc |
| 13 | Flagellin + | cgggggatccactagttctagagcggccggccgccaccgcggtggagct |
| | Somatotropin | ccagcttttgttccctttagtgagggttaattgcgcgcttggcgtaatc |
| | Italics: NR promoter | atggtcatagctgtttcctgtgtgaaattgttatccgctcacaattcca |
| | and terminator | cacaacatacgagccggaagcataaagtgtaaagcctggggtgcctaat |
| | CAPITAL | gagtgagctaactcacattaattgcgttgcgctcactgcccgctttcca |
| | UNDERLINED = | gtcgggaaacctgtcgtgccagctgcattaatgaatcggccaacgcgcg |
| | Flagellin | gggagaggcggtttgcgtattgggcgctcttccgcttcctcgctcactg |
| | Bold = | actcgctgcgctcggtcgttcggctgcggcgagcggtatcagctcactc |
| | Somatotropin | aaaggcggtaatacggttatccacagaatcaggggataacgcaggaaag |
| | Lower underlined = | aacatgtgagcaaaaggccagcaaaaggccaggaaccgtaaaaaggccg |
| | eGFP | cgttgctggcgtttttccataggctccgcccccctgacgagcatcacaa |
| | | aaatcgacgctcaagtcagaggtggcgaaacccgacaggactataaaga |
| | | taccaggcgtttccccctggaagctccctcgtgcgctctcctgttccga |
| | | ccctgccgcttaccggatacctgtccgcctttctcccttcgggaagcgt |
| | | ggcgctttctcatagctcacgctgtaggtatctcagttcggtgtaggtc |
| | | gttcgctccaagctgggctgtgtgcacgaaccccccgttcagcccgacc |
| | | gctgcgccttatccggtaactatcgtcttgagtccaacccggtaagaca |
| | | cgacttatcgccactggcagcagccactggtaacaggattagcagagcg |
| | | aggtatgtaggcggtgctacagagttcttgaagtggtggcctaactacg |
| | | gctacactagaaggacagtatttggtatctgcgctctgctgaagccagt |
| | | taccttcggaaaaagagttggtagctcttgatccggcaaacaaaccacc |
| | | gctggtagcggtggtttttttgtttgcaagcagcagattacgcgcagaa |
| | | aaaaaggatctcaagaagatcctttgatcttttctacggggtctgacgc |
| | | tcagtggaacgaaaactcacgttaagggattttggtcatgagattatca |
| | | aaaaggatcttcacctagatccttttaaattaaaaatgaagttttaaat |
| | | caatctaaagtatatatgagtaaacttggtctgacagttaccaatgctt |
| | | aatcagtgaggcacctatctcagcgatctgtctatttcgttcatccata |
| | | gttgcctgactccccgtcgtgtagataactacgatacgggagggcttac |
| | | catctggccccagtgctgcaatgataccgcgagacccacgctcaccggc |
| | | tccagatttatcagcaataaaccagccagccggaagggccgagcgcaga |
| | | agtggtcctgcaactttatccgcctccatccagtctattaattgttgcc |
| | | gggaagctagagtaagtagttcgccagttaatagtttgcgcaacgttgt |
| | | tgccattgctacaggcatcgtggtgtcacgctcgtcgtttggtatggct |
| | | tcattcagctccggttcccaacgatcaaggcgagttacatgatccccca |
| | | tgttgtgcaaaaaagcggttagctccttcggtcctccgatcgttgtcag |
| | | aagtaagttggccgcagtgttatcactcatggttatggcagcactgcat |
| | | aattctcttactgtcatgccatccgtaagatgcttttctgtgactggtg |
| | | agtactcaaccaagtcattctgagaatagtgtatgcggcgaccgagttg |
| | | ctcttgcccggcgtcaatacgggataataccgcgccacatagcagaact |
| | | ttaaaagtgctcatcattggaaaacgttcttcggggcgaaaactctcaa |
| | | ggatcttaccgctgttgagatccagttcgatgtaacccactcgtgcacc |
| | | caactgatcttcagcatcttttactttcaccagcgtttctgggtgagca |
| | | aaaacaggaaggcaaaatgccgcaaaaaagggaataagggcgacacgga |
| | | aatgttgaatactcatactcttcctttttcaatattattgaagcattta |
| | | tcagggttattgtctcatgagcggatacatatttgaatgtatttagaaa |
| | | aataaacaaataggggttccgcgcacatttccccgaaaagtgccaccta |
| | | aattgtaagcgttaatattttgttaaaattcgcgttaaatttttgttaa |
| | | atcagctcattttttaaccaataggccgaaatcggcaaaatcccttata |
| | | aatcaaaagaatagaccgagatagggttgagtgttgttccagtttggaa |
| | | caagagtccactattaaagaacgtggactccaacgtcaaagggcgaaaa |
| | | accgtctatcagggcgatggcccactacgtgaaccatcaccctaatcaa |
| | | gttttttggggtcgaggtgccgtaaagcactaaatcggaaccctaaagg |
| | | gagcccccgatttagagcttgacggggaaagccggcgaacgtggcgaga |
| | | aaggaagggaagaaagcgaaaggagcgggcgctagggcgctggcaagtg |
| | | tagcggtcacgctgcgcgtaaccaccacacccgccgcgcttaatgcgcc |
| | | gctacagggcgcgtcccattcgccattcaggctgcgcaactgttgggaa |
| | | gggcgatcggtgcgggcctcttcgctattacgccagctggcgaaagggg |
| | | gatgtgctgcaaggcgattaagttgggtaacgccagggttttcccagtc |
| | | acgacgttgtaaaacgacggccagtgagcgcgcgtaatacgactcacta |
| | | tagggcgaattgggtaccgggccccccctcgaggcagttgcctaccgaa |
| | | aagctcacggagatgaagaatggacgccctttcattttgaagcggagga |
| | | tatggaagaagtgacggggagtaacggagctcattatgctgctcatacg |
| | | ggaaatgttgagactttgatgcatatcatccagaatgagagaattgata |
| | | tggtgcatgaaacggatgggaatggatggctgcctattcatgaggctgc |
| | | acgaagtggtcatgtggagatcgtgcagattctgattgagcatggggcg |
| | | gatgtggatagtaggacggaatggggggagggacattcggtgttgaaca |
| | | ttgcgacgcagtaccacgaggaggatagttcaatcattcagtacttgtt |
| | | gagtttgggggcgatgaatatcattccttggcctgaggaggaggagctt |
| | | tagatgcgacaacatgagatgttttaggagtgtgtggggtaggaaggag |
| | | aagtatctagtattgcaagggtcgatgtttggaagtcagacggatcaat |
| | | tacgatctatactggagttgcagtggacaagaacatgtgggacctcgat |
| | | aaaggatatggtgcttgggcgggtgtcatcagatacactgcctagaggg |
| | | caattgattgcggtatatgaaacgtgcttcttgttgctgtaaaaataac |
| | | tcgtcgaacaagtcatccgactagcgcctctggtgatcaaaccacgcac |
| | | gtacggattggtttgatggttatcctcggattggtatccttagaacatc |
| | | ttcgaaggttctaggagactacccgcaaaacggatggtatttcccccca |
| | | acatccaaaattggaatgccacgtcatctgcacttccggactctggcag |
| | | cagcaaaatggcgcgctcgaccacatcacggttctccaccctcttccga |
| | | cagcaccaacatctctttccggaagaaccaagctggaggatgaagatga |
| | | cgatgagaagaggtgtggacggggtgaaaagaggctcgggaaggctttt |
| | | cggcaagcacagagcttttgcacataacaacctctcacaattcaccaac |
| | | ccatccaattgacagcctccatcttttaacatttcaacacaaaggaacc |
| | | aacagatatcataatcATGGCACAAGTCATCAACACCAACTCTCTCTCT |
| | | CTCTTGACCCAGAACAACCTCAACAAGTCCCAGTCCGCTCTCGGAACCG |
| | | CTATCGAGCGTCTCTCTTCCGGACTCCGTATCAACTCTGCTAAGGACGA |
| | | TGCTGCAGGACAGGCTATCGCTAACCGTTTCACCGCTAACATCAAGGGA |
| | | CTCACTCAGGCTTCCCGTAACGCTAACGACGGAATCTCCATCGCTCAGA |
| | | CCACTGAGGGAGCTCTCAACGAGATCAACAACAACCTCCAGCGTGTGCG |
| | | TGAGCTCGCTGTTCAGTCTGCTAACTCTACCAACTCCCAGTCTGACCTC |
| | | GACTCCATCCAGGCTGAGATCACCCAGCGTCTCAACGAGATCGACCGTG |
| | | TGTCCGGACAGACTCAGTTCAACGGAGTGAAGGTCCTCGCTCAGGACAA |
| | | CACCCTCACCATCCAGGTTGGAGCCAACGACGGAGAGACTATCGATATC |
| | | GATCTCAAGCAGATCAACTCTCAGACCCTCGGACTCGATACCCTCAACG |
| | | TGCAACAAAAGTACAAGGTCTCTGATACCGCTGCAACTGTTACCGGATA |
| | | CGCCGATACTACCATCGCTCTCGACAACAGTACTTTCAAGGCCTCTGCT |
| | | ACTGGACTCGGAGGAACTGACCAGAAGATCGATGGAGATCTCAAGTTCG |
| | | ATGATACCACTGGAAAGTACTACGCCAAGGTTACCGTTACCGGAGGAAC |
| | | TGGAAAGGATGGATACTACGAGGTTTCCGTTGATAAGACCAACGGAAAG |
| | | GTGACTCTCGCTGGAGGAGCTACTTCCCCACTCACCGGAGGACTCCCTG |
| | | CTACCGCAACTGAGGATGTGAAGAACGTGCAAGTTGCAAACGCTGATTT |
| | | GACCGAGGCTAAGGCCGCATTGACCGCAGCAGGAGTTACCGGAACCGCA |
| | | TCTGTTGTTAAGATGTCTTACACTGATAACAACGGAAAGACTATCGATG |
| | | GAGGACTCGCAGTTAAGGTGGGAGATGATTACTACTCTGCAACTCAAAA |
| | | CAAGGATGGATCCATCAGTATCAACACTACCAAGTACACTGCAGATGAC |
| | | GGAACCTCCAAGACTGCACTCAACAAGCTCGGAGGAGCAGACGGAAAGA |
| | | CCGAGGTTGTTTCTATCGGAGGAAAGACTTACGCTGCAAGTAAGGCCGA |
| | | GGGACACAACTTCAAGGCACAGCCTGATCTCGCTGAGGCTGCTGCTACC |
| | | ACCACCGAGAACCCACTCCAGAAGATCGATGCTGCTTTGGCACAGGTTG |
| | | ACACCCTCCGTTCTGACCTCGGAGCTGTGCAGAACCGTTTCAACTCCGC |
| | | TATCACCAACCTCGGAAACACCGTGAACAACCTCACTTCTGCCCGTTCT |
| | | CGTATCGAGGATTCCGACTACGCTACCGAGGTTTCCAACATGTCTCGTG |
| | | CTCAGATCCTCCAGCAGGCCGGAACCTCCGTTCTCGCTCAGGCTAACCA |
| | | GGTTCCACAAAACGTCCTCTCTCTCCTCCGTATGCAACCAATCACCGAG |
| | | AACCAGCGTTTGTTCTCCATCGCTGTCGGACGTGTGCAATACTTGCACC |
| | | TCGTCGCCAAGAAGCTCTTCTCCGATTTCGAGAACAGTTTGCAATTGGA |
| | | GGACCAGCGTTTGCTCAACAAGATCGCATCTAAGGAGTTCTGCCACTCT |
| | | GATAACTTCTTGTCCCCTATCGACAAGCATGAGACTCAAGGAAGTTCTG |
| | | TCCAGAAGTTGCTCTCTGTGTCCTACCGTTTGATCGAGAGTTGGGAGTT |
| | | CTTCTCTCGTTTCCTCGTGGCTTCTTTCGCCGTTCGTACCCAGGTCACT |
| | | TCTAAGCTCTCTGAGTTGAAGATGGGATTGCTCAAGTTGATCGAGGCAA |
| | | ACCAAGATGGAGCTGGAGGATTCTCCGAGTCCAGTGTTTTGCAGCTCAC |
| | | CCCATACGGAAACAGTGAGTTGTTCGCCTGTTTCAAGAAGGACATGCAT |
| | | AAGGTTGAGACTTACTTGACTGTCGCCAAGTGTCGTCTCTTCCCTGAGG |
| | | CAAACTGCACTCTCatggtgagcaagggcgaggagctgttcaccggggt |
| | | ggtgcccatcctggtcgagctggacggcgacgtaaacggccacaagttc |
| | | agcgtgtccggcgagggcgagggcgatgccacctacggcaagctgaccc |
| | | tgaagttcatctgcaccaccggcaagctgcccgtgccctggcccaccct |
| | | cgtgaccaccctgacctacggcgtgcagtgcttcagccgctaccccgac |
| | | cacatgaagcagcacgacttcttcaagtccgccatgcccgaaggctacg |
| | | tccaggagcgcaccatcttcttcaaggacgacggcaactacaagacccg |
| | | cgccgaggtgaagttcgagggcgacaccctggtgaaccgcatcgagctg |
| | | aagggcatcgacttcaaggaggacggcaacatcctggggcacaagctgg |
| | | agtacaactacaacagccacaacgtctatatcatggccgacaagcagaa |
| | | gaacggcatcaaggtgaacttcaagatccgccacaacatcgaggacggc |
| | | agcgtgcagctcgccgaccactaccagcagaacacccccatcggcgacg |
| | | gccccgtgctgctgcccgacaaccactacctgagcacccagtccgccct |
| | | gagcaaagaccccaacgagaagcgcgatcacatggtcctgctggagttc |
| | | gtgaccgccgccgggatcactctcggcatggacgagctgtacaagtaag |
| | | cggccgctgaggatgctcatcgttttgactttgttggatgacgcctctt |
| | | gtacatactgattagacatgggagtgttggttgaattgtagatagttta |
| | | caccggctgttaactgaagagatactattacactattcatcatcattcc |
| | | aattgatacgtcgccgccacgcactgctcatccatagaaccctcagaca |
| | | cacttcacctcaaaggttcgtatctccatcggctccaaagaaaccacac |
| | | tgttgccttgctccagcttacgactatcctccttgaaatcatcatcgtt |
| | | ccaccactgaaactgtctctgttcccactcatctcttctctgattacca |
| | | gagagtgtcgtttccgtgatggagactactttgcgagaaggaaacaaga |
| | | cgccaagatccacctcaaccggaagggagtattgatgactttctcccac |
| | | tccgtactgatgcccaagcctgacaagataggtgtcgtcttgctctagt |
| | | gatgcaaacgttaccagcatgatattatcaggcaatgcatcc |
| 14 | Flagellin +. Vibrio | cgggggatccactagttctagagcggccggccgccaccgcggtggagct |
| | parahaemolyticus | ccagcttttgttccctttagtgagggttaattgcgcgcttggcgtaatc |
| | pirA^{VP} | atggtcatagctgtttcctgtgtgaaattgttatccgctcacaattcca |
| | Italics: NR promoter | cacaacatacgagccggaagcataaagtgtaaagcctggggtgcctaat |
| | and terminator | gagtgagctaactcacattaattgcgttgcgctcactgcccgctttcca |
| | CAPITAL | gtcgggaaacctgtcgtgccagctgcattaatgaatcggccaacgcgcg |
| | UNDERLINED | gggagaggcggtttgcgtattgggcgctcttccgcttcctcgctcactg |
| | Flagellin | actcgctgcgctcggtcgttcggctgcggcgagcggtatcagctcactc |
| | Bold = pirA^{VP} | aaaggcggtaatacggttatccacagaatcaggggataacgcaggaaag |
| | Lower underlined = | aacatgtgagcaaaaggccagcaaaaggccaggaaccgtaaaaaggccg |
| | eGFP | cgttgctggcgtttttccataggctccgcccccctgacgagcatcacaa |
| | | aaatcgacgctcaagtcagaggtggcgaaacccgacaggactataaaga |
| | | taccaggcgtttccccctggaagctccctcgtgcgctctcctgttccga |
| | | ccctgccgcttaccggatacctgtccgcctttctcccttcgggaagcgt |
| | | ggcgctttctcatagctcacgctgtaggtatctcagttcggtgtaggtc |
| | | gttcgctccaagctgggctgtgtgcacgaaccccccgttcagcccgacc |
| | | gctgcgccttatccggtaactatcgtcttgagtccaacccggtaagaca |
| | | cgacttatcgccactggcagcagccactggtaacaggattagcagagcg |
| | | aggtatgtaggcggtgctacagagttcttgaagtggtggcctaactacg |
| | | gctacactagaaggacagtatttggtatctgcgctctgctgaagccagt |
| | | taccttcggaaaaagagttggtagctcttgatccggcaaacaaaccacc |
| | | gctggtagcggtggtttttttgtttgcaagcagcagattacgcgcagaa |
| | | aaaaaggatctcaagaagatcctttgatcttttctacggggtctgacgc |
| | | tcagtggaacgaaaactcacgttaagggattttggtcatgagattatca |
| | | aaaaggatcttcacctagatccttttaaattaaaaatgaagttttaaat |
| | | caatctaaagtatatatgagtaaacttggtctgacagttaccaatgctt |
| | | aatcagtgaggcacctatctcagcgatctgtctatttcgttcatccata |
| | | gttgcctgactccccgtcgtgtagataactacgatacgggagggcttac |
| | | catctggccccagtgctgcaatgataccgcgagacccacgctcaccggc |
| | | tccagatttatcagcaataaaccagccagccggaagggccgagcgcaga |
| | | agtggtcctgcaactttatccgcctccatccagtctattaattgttgcc |
| | | gggaagctagagtaagtagttcgccagttaatagtttgcgcaacgttgt |
| | | tgccattgctacaggcatcgtggtgtcacgctcgtcgtttggtatggct |
| | | tcattcagctccggttcccaacgatcaaggcgagttacatgatccccca |
| | | tgttgtgcaaaaaagcggttagctccttcggtcctccgatcgttgtcag |
| | | aagtaagttggccgcagtgttatcactcatggttatggcagcactgcat |
| | | aattctcttactgtcatgccatccgtaagatgcttttctgtgactggtg |
| | | agtactcaaccaagtcattctgagaatagtgtatgcggcgaccgagttg |
| | | ctcttgcccggcgtcaatacgggataataccgcgccacatagcagaact |
| | | ttaaaagtgctcatcattggaaaacgttcttcggggcgaaaactctcaa |
| | | ggatcttaccgctgttgagatccagttcgatgtaacccactcgtgcacc |
| | | caactgatcttcagcatcttttactttcaccagcgtttctgggtgagca |
| | | aaaacaggaaggcaaaatgccgcaaaaaagggaataagggcgacacgga |
| | | aatgttgaatactcatactcttcctttttcaatattattgaagcattta |
| | | tcagggttattgtctcatgagcggatacatatttgaatgtatttagaaa |
| | | aataaacaaataggggttccgcgcacatttccccgaaaagtgccaccta |
| | | aattgtaagcgttaatattttgttaaaattcgcgttaaatttttgttaa |
| | | atcagctcattttttaaccaataggccgaaatcggcaaaatcccttata |
| | | aatcaaaagaatagaccgagatagggttgagtgttgttccagtttggaa |
| | | caagagtccactattaaagaacgtggactccaacgtcaaagggcgaaaa |
| | | accgtctatcagggcgatggcccactacgtgaaccatcaccctaatcaa |
| | | gttttttggggtcgaggtgccgtaaagcactaaatcggaaccctaaagg |
| | | gagcccccgatttagagcttgacggggaaagccggcgaacgtggcgaga |
| | | aaggaagggaagaaagcgaaaggagcgggcgctagggcgctggcaagtg |
| | | tagcggtcacgctgcgcgtaaccaccacacccgccgcgcttaatgcgcc |
| | | gctacagggcgcgtcccattcgccattcaggctgcgcaactgttgggaa |
| | | gggcgatcggtgcgggcctcttcgctattacgccagctggcgaaagggg |
| | | gatgtgctgcaaggcgattaagttgggtaacgccagggttttcccagtc |
| | | acgacgttgtaaaacgacggccagtgagcgcgcgtaatacgactcacta |
| | | tagggcgaattgggtaccgggcccccccagagggcaattgattgcggta |
| | | tatgaaacgtgcttcttgttgctgtaaaaataactcgtcgaacaagtca |
| | | tccgactagcgcctctggtgatcaaaccacgcacgtacggattggtttg |
| | | atggttatcctcggattggtatccttagaacatcttcgaaggttctagg |
| | | agactacccgcaaaacggatggtatttccccccaacatccaaaattgga |
| | | atgccacgtcatctgcacttccggactctggcagcagcaaaatggcgcg |
| | | ctcgaccacatcacggttctccaccctcttccgacagcaccaacatctc |
| | | tttccggaagaaccaagctggaggatgaagatgacgatgagaagaggtg |
| | | tggacggggtgaaaagaggctcgggaaggcttttcggcaagcacagagc |
| | | ttttgcacataacaacctctcacaattcaccaacccatccaattgacag |
| | | cctccatcttttaacatttcaacacaaaggaaccaacagatatcataat |
| | | CATGGCACAAGTCATCAACACCAACTCTCTCTCTCTCTTGACCCAGAAC |
| | | AACCTCAACAAGTCCCAGTCCGCTCTCGGAACCGCTATCGAGCGTCTCT |
| | | CTTCCGGACTCCGTATCAACTCTGCTAAGGACGATGCTGCAGGACAGGC |
| | | TATCGCTAACCGTTTCACCGCTAACATCAAGGGACTCACTCAGGCTTCC |
| | | CGTAACGCTAACGACGGAATCTCCATCGCTCAGACCACTGAGGGAGCTC |
| | | TCAACGAGATCAACAACAACCTCCAGCGTGTGCGTGAGCTCGCTGTTCA |
| | | GTCTGCTAACTCTACCAACTCCCAGTCTGACCTCGACTCCATCCAGGCT |
| | | GAGATCACCCAGCGTCTCAACGAGATCGACCGTGTGTCCGGACAGACTC |
| | | AGTTCAACGGAGTGAAGGTCCTCGCTCAGGACAACACCCTCACCATCCA |
| | | GGTTGGAGCCAACGACGGAGAGACTATCGATATCGATCTCAAGCAGATC |
| | | AACTCTCAGACCCTCGGACTCGATACCCTCAACGTGCAACAAAAGTACA |
| | | AGGTCTCTGATACCGCTGCAACTGTTACCGGATACGCCGATACTACCAT |
| | | CGCTCTCGACAACAGTACTTTCAAGGCCTCTGCTACTGGACTCGGAGGA |
| | | ACTGACCAGAAGATCGATGGAGATCTCAAGTTCGATGATACCACTGGAA |
| | | AGTACTACGCCAAGGTTACCGTTACCGGAGGAACTGGAAAGGATGGATA |
| | | CTACGAGGTTTCCGTTGATAAGACCAACGGAAAGGTGACTCTCGCTGGA |
| | | GGAGCTACTTCCCCACTCACCGGAGGACTCCCTGCTACCGCAACTGAGG |
| | | ATGTGAAGAACGTGCAAGTTGCAAACGCTGATTTGACCGAGGCTAAGGC |
| | | CGCATTGACCGCAGCAGGAGTTACCGGAACCGCATCTGTTGTTAAGATG |
| | | TCTTACACTGATAACAACGGAAAGACTATCGATGGAGGACTCGCAGTTA |
| | | AGGTGGGAGATGATTACTACTCTGCAACTCAAAACAAGGATGGATCCAT |
| | | CAGTATCAACACTACCAAGTACACTGCAGATGACGGAACCTCCAAGACT |
| | | GCACTCAACAAGCTCGGAGGAGCAGACGGAAAGACCGAGGTTGTTTCTA |
| | | TCGGAGGAAAGACTTACGCTGCAAGTAAGGCCGAGGGACACAACTTCAA |
| | | GGCACAGCCTGATCTCGCTGAGGCTGCTGCTACCACCACCGAGAACCCA |
| | | CTCCAGAAGATCGATGCTGCTTTGGCACAGGTTGACACCCTCCGTTCTG |
| | | ACCTCGGAGCTGTGCAGAACCGTTTCAACTCCGCTATCACCAACCTCGG |
| | | AAACACCGTGAACAACCTCACTTCTGCCCGTTCTCGTATCGAGGATTCC |
| | | GACTACGCTACCGAGGTTTCCAACATGTCTCGTGCTCAGATCCTCCAGC |
| | | AGGCCGGAACCTCCGTTCTCGCTCAGGCTAACCAGGTTCCACAAAACGT |
| | | CCTCTCTCTCCTCCGTATGTCTAACAACATCAAGCATGAGACTGATTAC |
| | | TCTCACGATTGGACCGTCGAGCCAAACGGAGGAGTGACCGAGGTGGATT |
| | | CTAAGCATACCCCAATCATCCCTGAGGTGGGACGTTCTGTCGATATCGA |
| | | GAACACTGGACGTGGAGAGTTGACCATCCAATACCAATGGGGAGCCCCA |
| | | TTCATGGCTGGAGGATGGAAGGTTGCCAAGTCTCATGTCGTGCAACGTG |
| | | ATGAGACCTACCATTTGCAACGTCCTGATAACGCTTTCTACCACCAACG |
| | | TATCGTTGTGATCAACAACGGAGCCTCTCGTGGATTCTGTACCATCTAC |
| | | TACCATatggtgagcaagggcgaggagctgttcaccggggtggtgccca |
| | | tcctggtcgagctggacggcgacgtaaacggccacaagttcagcgtgtc |
| | | cggcgagggcgagggcgatgccacctacggcaagctgaccctgaagttc |
| | | atctgcaccaccggcaagctgcccgtgccctggcccaccctcgtgacca |
| | | ccctgacctacggcgtgcagtgcttcagccgctaccccgaccacatgaa |
| | | gcagcacgacttcttcaagtccgccatgcccgaaggctacgtccaggag |
| | | cgcaccatcttcttcaaggacgacggcaactacaagacccgcgccgagg |
| | | tgaagttcgagggcgacaccctggtgaaccgcatcgagctgaagggcat |
| | | cgacttcaaggaggacggcaacatcctggggcacaagctggagtacaac |
| | | tacaacagccacaacgtctatatcatggccgacaagcagaagaacggca |
| | | tcaaggtgaacttcaagatccgccacaacatcgaggacggcagcgtgca |
| | | gctcgccgaccactaccagcagaacacccccatcggcgacggccccgtg |
| | | ctgctgcccgacaaccactacctgagcacccagtccgccctgagcaaag |
| | | accccaacgagaagcgcgatcacatggtcctgctggagttcgtgaccgc |
| | | cgccgggatcactctcggcatggacgagctgtacaagtaagcggccgct |
| | | gaggatgctcatcgttttgactttgttggatgacgcctcttgtacatac |
| | | tgattagacatgggagtgttggttgaattgtagatagtttacaccggct |
| | | gttaactgaagagatactattacactattcatcatcattccaattgata |
| | | cgtcgccgccacgcactgctcatccatagaaccctcagacacacttcac |
| | | ctcaaaggttcgtatctccatcggctccaaagaaaccacactgttgcct |
| | | tgctccagcttacgactatcctccttgaaatcatcatcgttccaccact |
| | | gaaactgtctctgttcccactcatctcttctctgattaccagagagtgt |
| | | cgtttccgtgatggagactactttgcgagaaggaaacaagacgccaaga |
| | | tccacctcaaccggaagggagtattgatgactttctcccactccgtact |
| | | gatgcccaagcctgacaagataggtgtcgtcttgctctagtgatgcaaa |
| | | cgttaccagcatgatattatcaggcaatgcatcc |
| 15 | Flagellin + Vibrio | cgggggatccactagttctagagcggccggccgccaccgcggtggagct |
| | parahaemolyticus | ccagcttttgttccctttagtgagggttaattgcgcgcttggcgtaatc |
| | pirB^{VP} | atggtcatagctgtttcctgtgtgaaattgttatccgctcacaattcca |
| | Italics: NR promoter | cacaacatacgagccggaagcataaagtgtaaagcctggggtgcctaat |
| | and terminator | gagtgagctaactcacattaattgcgttgcgctcactgcccgctttcca |
| | CAPITAL | gtcgggaaacctgtcgtgccagctgcattaatgaatcggccaacgcgcg |
| | UNDERLINED | gggagaggcggtttgcgtattgggcgctcttccgcttcctcgctcactg |
| | Flagellin | actcgctgcgctcggtcgttcggctgcggcgagcggtatcagctcactc |
| | Bold = pirB^{VP} | aaaggcggtaatacggttatccacagaatcaggggataacgcaggaaag |
| | Lower underlined = | aacatgtgagcaaaaggccagcaaaaggccaggaaccgtaaaaaggccg |
| | eGFP | cgttgctggcgtttttccataggctccgcccccctgacgagcatcacaa |
| | | aaatcgacgctcaagtcagaggtggcgaaacccgacaggactataaaga |
| | | taccaggcgtttccccctggaagctccctcgtgcgctctcctgttccga |
| | | ccctgccgcttaccggatacctgtccgcctttctcccttcgggaagcgt |
| | | ggcgctttctcatagctcacgctgtaggtatctcagttcggtgtaggtc |
| | | gttcgctccaagctgggctgtgtgcacgaaccccccgttcagcccgacc |
| | | gctgcgccttatccggtaactatcgtcttgagtccaacccggtaagaca |
| | | cgacttatcgccactggcagcagccactggtaacaggattagcagagcg |
| | | aggtatgtaggcggtgctacagagttcttgaagtggtggcctaactacg |
| | | gctacactagaaggacagtatttggtatctgcgctctgctgaagccagt |
| | | taccttcggaaaaagagttggtagctcttgatccggcaaacaaaccacc |
| | | gctggtagcggtggtttttttgtttgcaagcagcagattacgcgcagaa |
| | | aaaaaggatctcaagaagatcctttgatcttttctacggggtctgacgc |
| | | tcagtggaacgaaaactcacgttaagggattttggtcatgagattatca |
| | | aaaaggatcttcacctagatccttttaaattaaaaatgaagttttaaat |
| | | caatctaaagtatatatgagtaaacttggtctgacagttaccaatgctt |
| | | aatcagtgaggcacctatctcagcgatctgtctatttcgttcatccata |
| | | gttgcctgactccccgtcgtgtagataactacgatacgggagggcttac |
| | | catctggccccagtgctgcaatgataccgcgagacccacgctcaccggc |
| | | tccagatttatcagcaataaaccagccagccggaagggccgagcgcaga |
| | | agtggtcctgcaactttatccgcctccatccagtctattaattgttgcc |
| | | gggaagctagagtaagtagttcgccagttaatagtttgcgcaacgttgt |
| | | tgccattgctacaggcatcgtggtgtcacgctcgtcgtttggtatggct |
| | | tcattcagctccggttcccaacgatcaaggcgagttacatgatccccca |
| | | tgttgtgcaaaaaagcggttagctccttcggtcctccgatcgttgtcag |
| | | aagtaagttggccgcagtgttatcactcatggttatggcagcactgcat |
| | | aattctcttactgtcatgccatccgtaagatgcttttctgtgactggtg |
| | | agtactcaaccaagtcattctgagaatagtgtatgcggcgaccgagttg |
| | | ctcttgcccggcgtcaatacgggataataccgcgccacatagcagaact |
| | | ttaaaagtgctcatcattggaaaacgttcttcggggcgaaaactctcaa |
| | | ggatcttaccgctgttgagatccagttcgatgtaacccactcgtgcacc |
| | | caactgatcttcagcatcttttactttcaccagcgtttctgggtgagca |
| | | aaaacaggaaggcaaaatgccgcaaaaaagggaataagggcgacacgga |
| | | aatgttgaatactcatactcttcctttttcaatattattgaagcattta |
| | | tcagggttattgtctcatgagcggatacatatttgaatgtatttagaaa |
| | | aataaacaaataggggttccgcgcacatttccccgaaaagtgccaccta |
| | | aattgtaagcgttaatattttgttaaaattcgcgttaaatttttgttaa |
| | | atcagctcattttttaaccaataggccgaaatcggcaaaatcccttata |
| | | aatcaaaagaatagaccgagatagggttgagtgttgttccagtttggaa |
| | | caagagtccactattaaagaacgtggactccaacgtcaaagggcgaaaa |
| | | accgtctatcagggcgatggcccactacgtgaaccatcaccctaatcaa |
| | | gttttttggggtcgaggtgccgtaaagcactaaatcggaaccctaaagg |
| | | gagcccccgatttagagcttgacggggaaagccggcgaacgtggcgaga |
| | | aaggaagggaagaaagcgaaaggagcgggcgctagggcgctggcaagtg |
| | | tagcggtcacgctgcgcgtaaccaccacacccgccgcgcttaatgcgcc |
| | | gctacagggcgcgtcccattcgccattcaggctgcgcaactgttgggaa |
| | | gggcgatcggtgcgggcctcttcgctattacgccagctggcgaaagggg |
| | | gatgtgctgcaaggcgattaagttgggtaacgccagggttttcccagtc |
| | | acgacgttgtaaaacgacggccagtgagcgcgcgtaatacgactcacta |
| | | tagggcgaattgggtaccgggcccccccagagggcaattgattgcggta |
| | | tatgaaacgtgcttcttgttgctgtaaaaataactcgtcgaacaagtca |
| | | tccgactagcgcctctggtgatcaaaccacgcacgtacggattggtttg |
| | | atggttatcctcggattggtatccttagaacatcttcgaaggttctagg |
| | | agactacccgcaaaacggatggtatttccccccaacatccaaaattgga |
| | | atgccacgtcatctgcacttccggactctggcagcagcaaaatggcgcg |
| | | ctcgaccacatcacggttctccaccctcttccgacagcaccaacatctc |
| | | tttccggaagaaccaagctggaggatgaagatgacgatgagaagaggtg |
| | | tggacggggtgaaaagaggctcgggaaggcttttcggcaagcacagagc |
| | | ttttgcacataacaacctctcacaattcaccaacccatccaattgacag |
| | | cctccatcttttaacatttcaacacaaaggaaccaacagatatcataat |
| | | cATGGCACAAGTCATCAACACCAACTCTCTCTCTCTCTTGACCCAGAAC |
| | | AACCTCAACAAGTCCCAGTCCGCTCTCGGAACCGCTATCGAGCGTCTCT |
| | | CTTCCGGACTCCGTATCAACTCTGCTAAGGACGATGCTGCAGGACAGGC |
| | | TATCGCTAACCGTTTCACCGCTAACATCAAGGGACTCACTCAGGCTTCC |
| | | CGTAACGCTAACGACGGAATCTCCATCGCTCAGACCACTGAGGGAGCTC |
| | | TCAACGAGATCAACAACAACCTCCAGCGTGTGCGTGAGCTCGCTGTTCA |
| | | GTCTGCTAACTCTACCAACTCCCAGTCTGACCTCGACTCCATCCAGGCT |
| | | GAGATCACCCAGCGTCTCAACGAGATCGACCGTGTGTCCGGACAGACTC |
| | | AGTTCAACGGAGTGAAGGTCCTCGCTCAGGACAACACCCTCACCATCCA |
| | | GGTTGGAGCCAACGACGGAGAGACTATCGATATCGATCTCAAGCAGATC |
| | | AACTCTCAGACCCTCGGACTCGATACCCTCAACGTGCAACAAAAGTACA |
| | | AGGTCTCTGATACCGCTGCAACTGTTACCGGATACGCCGATACTACCAT |
| | | CGCTCTCGACAACAGTACTTTCAAGGCCTCTGCTACTGGACTCGGAGGA |
| | | ACTGACCAGAAGATCGATGGAGATCTCAAGTTCGATGATACCACTGGAA |
| | | AGTACTACGCCAAGGTTACCGTTACCGGAGGAACTGGAAAGGATGGATA |
| | | CTACGAGGTTTCCGTTGATAAGACCAACGGAAAGGTGACTCTCGCTGGA |
| | | GGAGCTACTTCCCCACTCACCGGAGGACTCCCTGCTACCGCAACTGAGG |
| | | ATGTGAAGAACGTGCAAGTTGCAAACGCTGATTTGACCGAGGCTAAGGC |
| | | CGCATTGACCGCAGCAGGAGTTACCGGAACCGCATCTGTTGTTAAGATG |
| | | TCTTACACTGATAACAACGGAAAGACTATCGATGGAGGACTCGCAGTTA |
| | | AGGTGGGAGATGATTACTACTCTGCAACTCAAAACAAGGATGGATCCAT |
| | | CAGTATCAACACTACCAAGTACACTGCAGATGACGGAACCTCCAAGACT |
| | | GCACTCAACAAGCTCGGAGGAGCAGACGGAAAGACCGAGGTTGTTTCTA |
| | | TCGGAGGAAAGACTTACGCTGCAAGTAAGGCCGAGGGACACAACTTCAA |
| | | GGCACAGCCTGATCTCGCTGAGGCTGCTGCTACCACCACCGAGAACCCA |
| | | CTCCAGAAGATCGATGCTGCTTTGGCACAGGTTGACACCCTCCGTTCTG |
| | | ACCTCGGAGCTGTGCAGAACCGTTTCAACTCCGCTATCACCAACCTCGG |
| | | AAACACCGTGAACAACCTCACTTCTGCCCGTTCTCGTATCGAGGATTCC |
| | | GACTACGCTACCGAGGTTTCCAACATGTCTCGTGCTCAGATCCTCCAGC |
| | | AGGCCGGAACCTCCGTTCTCGCTCAGGCTAACCAGGTTCCACAAAACGT |
| | | CCTCTCTCTCCTCCGTATGACTAACGAGTACGTTGTGACTATGTCTTCT |
| | | CTCACTGAGTTCAACCCAAACAACGCTCGTAAGTCTTACCTCTTCGATA |
| | | ACTACGAGGTTGATCCTAACTACGCCTTCAAGGCCATGGTTTCTTTCGG |
| | | ATTGTCTAACATCCCATACGCCGGAGGATTCCTCTCTACTTTGTGGAAC |
| | | ATCTTCTGGCCAAACACTCCAAACGAGCCTGATATCGAGAACATCTGGG |
| | | AGCAATTGCGTGATCGTATCCAAGATCTCGTTGATGAGTCTATCATCGA |
| | | TGCCATCAACGGAATCCTCGATTCTAAGATCAAGGAGACTCGTGATAAG |
| | | ATCCAAGATATCAACGAGACCATCGAGAACTTCGGATACGCTGCCGCTA |
| | | AGGATGATTACATCGGACTCGTGACTCACTACTTGATCGGATTGGAGGA |
| | | GAACTTCAAGCGTGAGCTCGATGGAGATGAGTGGTTGGGATACGCTATC |
| | | TTGCCTCTCTTGGCCACCACTGTCTCTTTGCAAATCACCTACATGGCTT |
| | | GTGGATTGGATTACAAGGATGAGTTCGGATTCACTGATTCTGATGTCCA |
| | | TAAGCTCACCCGTAACATCGATAAGTTGTACGATGATGTTTCTTCTTAC |
| | | ATCACTGAGTTGGCCGCTTGGGCCGATAACGATTCTTACAACAACGCCA |
| | | ACCAAGATAACGTTTACGATGAGGTTATGGGAGCCCGTTCTTGGTGTAC |
| | | CGTGCATGGATTCGAGCATATGTTGATCTGGCAAAAGATCAAGGAGTTG |
| | | AAGAAGGTGGATGTGTTCGTGCACTCTAACCTCATCTCTTACTCTCCAG |
| | | CTGTGGGATTCCCATCTGGAAACTTCAACTACATCGCTACTGGAACCGA |
| | | GGATGAGATCCCTCAACCACTCAAGCCAAACATGTTCGGAGAGCGTCGT |
| | | AACCGTATCGTGAAGATCGAGTCTTGGAACTCTATCGAGATCCACTACT |
| | | ACAACCGTGTTGGACGTTTGAAGCTCACCTACGAGAACGGAGAGGTGGT |
| | | CGAGTTGGGAAAGGCCCATAAGTACGATGAGCATTACCAATCTATCGAG |
| | | CTCAACGGAGCCTACATCAAGTACGTTGATGTGATCGCCAACGGACCTG |
| | | AGGCTATCGATCGTATCGTCTTCCACTTCTCTGATGATCGTACCTTCGT |
| | | GGTTGGAGAGAACTCTGGAAAGCCATCTGTGCGTCTCCAACTCGAGGGA |
| | | CACTTCATCTGTGGAATGTTGGCTGATCAAGAGGGATCTGATAAGGTTG |
| | | CCGCTTTCTCTGTCGCTTACGAGCTCTTCCACCCAGATGAGTTCGGAAC |
| | | TGAGAAGatggtgagcaagggcgaggagctgttcaccggggtggtgccc |
| | | atcctggtcgagctggacggcgacgtaaacggccacaagttcagcgtgt |
| | | ccggcgagggcgagggcgatgccacctacggcaagctgaccctgaagtt |
| | | catctgcaccaccggcaagctgcccgtgccctggcccaccctcgtgacc |
| | | accctgacctacggcgtgcagtgcttcagccgctaccccgaccacatga |
| | | agcagcacgacttcttcaagtccgccatgcccgaaggctacgtccagga |
| | | gcgcaccatcttcttcaaggacgacggcaactacaagacccgcgccgag |
| | | gtgaagttcgagggcgacaccctggtgaaccgcatcgagctgaagggca |
| | | tcgacttcaaggaggacggcaacatcctggggcacaagctggagtacaa |
| | | ctacaacagccacaacgtctatatcatggccgacaagcagaagaacggc |
| | | atcaaggtgaacttcaagatccgccacaacatcgaggacggcagcgtgc |
| | | agctcgccgaccactaccagcagaacacccccatcggcgacggccccgt |
| | | gctgctgcccgacaaccactacctgagcacccagtccgccctgagcaaa |
| | | gaccccaacgagaagcgcgatcacatggtcctgctggagttcgtgaccg |
| | | ccgccgggatcactctcggcatggacgagctgtacaagtaagcggccgc |
| | | tgaggatgctcatcgttttgactttgttggatgacgcctcttgtacata |
| | | ctgattagacatgggagtgttggttgaattgtagatagtttacaccggc |
| | | tgttaactgaagagatactattacactattcatcatcattccaattgat |
| | | acgtcgccgccacgcactgctcatccatagaaccctcagacacacttca |
| | | cctcaaaggttcgtatctccatcggctccaaagaaaccacactgttgcc |
| | | ttgctccagcttacgactatcctccttgaaatcatcatcgttccaccac |
| | | tgaaactgtctctgttcccactcatctcttctctgattaccagagagtg |
| | | tcgtttccgtgatggagactactttgcgagaaggaaacaagacgccaag |
| | | atccacctcaaccggaagggagtattgatgactttctcccactccgtac |
| | | tgatgcccaagcctgacaagataggtgtcgtcttgctctagtgatgcaa |
| | | acgttaccagcatgatattatcaggcaatgcatcc |
| 16 | Flagellin + Vibrio | gggggatccactagttctagagcggccggccgccaccgcggtggagctc |
| | parahaemolyticus | cagcttttgttccctttagtgagggttaattgcgcgcttggcgtaatca |
| | Slp-YeaY family | tggtcatagctgtttcctgtgtgaaattgttatccgctcacaattccac |
| | lipoprotein | acaacatacgagccggaagcataaagtgtaaagcctggggtgcctaatg |
| | Italics: FCP | agtgagctaactcacattaattgcgttgcgctcactgcccgctttccag |
| | promoter and | tcgggaaacctgtcgtgccagctgcattaatgaatcggccaacgcgcgg |
| | terminator | ggagaggcggtttgcgtattgggcgctcttccgcttcctcgctcactga |
| | CAPITAL | ctcgctgcgctcggtcgttcggctgcggcgagcggtatcagctcactca |
| | UNDERLINED = | aaggcggtaatacggttatccacagaatcaggggataacgcaggaaaga |
| | Flagellin | acatgtgagcaaaaggccagcaaaaggccaggaaccgtaaaaaggccgc |
| | Bold = Slp-YeaY | gttgctggcgtttttccataggctccgcccccctgacgagcatcacaaa |
| | family lipoprotein | aatcgacgctcaagtcagaggtggcgaaacccgacaggactataaagat |
| | Lower underlined = | accaggcgtttccccctggaagctccctcgtgcgctctcctgttccgac |
| | eGFP | cctgccgcttaccggatacctgtccgcctttctcccttcgggaagcgtg |
| | | gcgctttctcatagctcacgctgtaggtatctcagttcggtgtaggtcg |
| | | ttcgctccaagctgggctgtgtgcacgaaccccccgttcagcccgaccg |
| | | ctgcgccttatccggtaactatcgtcttgagtccaacccggtaagacac |
| | | gacttatcgccactggcagcagccactggtaacaggattagcagagcga |
| | | ggtatgtaggcggtgctacagagttcttgaagtggtggcctaactacgg |
| | | ctacactagaaggacagtatttggtatctgcgctctgctgaagccagtt |
| | | accttcggaaaaagagttggtagctcttgatccggcaaacaaaccaccg |
| | | ctggtagcggtggtttttttgtttgcaagcagcagattacgcgcagaaa |
| | | aaaaggatctcaagaagatcctttgatcttttctacggggtctgacgct |
| | | cagtggaacgaaaactcacgttaagggattttggtcatgagattatcaa |
| | | aaaggatcttcacctagatccttttaaattaaaaatgaagttttaaatc |
| | | aatctaaagtatatatgagtaaacttggtctgacagttaccaatgctta |
| | | atcagtgaggcacctatctcagcgatctgtctatttcgttcatccatag |
| | | ttgcctgactccccgtcgtgtagataactacgatacgggagggcttacc |
| | | atctggccccagtgctgcaatgataccgcgagacccacgctcaccggct |
| | | ccagatttatcagcaataaaccagccagccggaagggccgagcgcagaa |
| | | gtggtcctgcaactttatccgcctccatccagtctattaattgttgccg |
| | | ggaagctagagtaagtagttcgccagttaatagtttgcgcaacgttgtt |
| | | gccattgctacaggcatcgtggtgtcacgctcgtcgtttggtatggctt |
| | | cattcagctccggttcccaacgatcaaggcgagttacatgatcccccat |
| | | gttgtgcaaaaaagcggttagctccttcggtcctccgatcgttgtcaga |
| | | agtaagttggccgcagtgttatcactcatggttatggcagcactgcata |
| | | attctcttactgtcatgccatccgtaagatgcttttctgtgactggtga |
| | | gtactcaaccaagtcattctgagaatagtgtatgcggcgaccgagttgc |
| | | tcttgcccggcgtcaatacgggataataccgcgccacatagcagaactt |
| | | taaaagtgctcatcattggaaaacgttcttcggggcgaaaactctcaag |
| | | gatcttaccgctgttgagatccagttcgatgtaacccactcgtgcaccc |
| | | aactgatcttcagcatcttttactttcaccagcgtttctgggtgagcaa |
| | | aaacaggaaggcaaaatgccgcaaaaaagggaataagggcgacacggaa |
| | | atgttgaatactcatactcttcctttttcaatattattgaagcatttat |
| | | cagggttattgtctcatgagcggatacatatttgaatgtatttagaaaa |
| | | ataaacaaataggggttccgcgcacatttccccgaaaagtgccacctaa |
| | | attgtaagcgttaatattttgttaaaattcgcgttaaatttttgttaaa |
| | | tcagctcattttttaaccaataggccgaaatcggcaaaatcccttataa |
| | | atcaaaagaatagaccgagatagggttgagtgttgttccagtttggaac |
| | | aagagtccactattaaagaacgtggactccaacgtcaaagggcgaaaaa |
| | | ccgtctatcagggcgatggcccactacgtgaaccatcaccctaatcaag |
| | | ttttttggggtcgaggtgccgtaaagcactaaatcggaaccctaaaggg |
| | | agcccccgatttagagcttgacggggaaagccggcgaacgtggcgagaa |
| | | aggaagggaagaaagcgaaaggagcgggcgctagggcgctggcaagtgt |
| | | agcggtcacgctgcgcgtaaccaccacacccgccgcgcttaatgcgccg |
| | | ctacagggcgcgtcccattcgccattcaggctgcgcaactgttgggaag |
| | | ggcgatcggtgcgggcctcttcgctattacgccagctggcgaaaggggg |
| | | atgtgctgcaaggcgattaagttgggtaacgccagggttttcccagtca |
| | | cgacgttgtaaaacgacggccagtgagcgcgcgtaatacgactcactat |
| | | agggcgaattgggtacgtaccgggccccccctcgaggtcgacggtatcg |
| | | ataagcttgcgctttttccgagaactccccataagtcaacggctccaat |
| | | caagaatgtatccgacaacggcgagcatagcaacacgtccgtctttgga |
| | | gtagaatcatcatgttgtggatgaatacacagatgaatgacattaaaag |
| | | catgaacatgttagagagtaggaggtagagattgatatggtagcattgc |
| | | gatgtttgtttttggtcagcatatgatgagtggataccaatatgatgaa |
| | | agttgaatctcgcgtttgagctcagcggtacgttattgatcgaaagtag |
| | | cctgatcaaaatccttggagagtacaagaggatcaaagaatccagtggg |
| | | ggcgataactccaagctcgttctcaaagaggcaatggaggtagaaactc |
| | | atcccagttgagaagaagtgaaggcagtggcggtggcgaaagcagaggc |
| | | aacgaggacagacttcctgtgggttgatgcaacgaatatttccagaagg |
| | | agaagtttagagagttgaaccgctacctacaatgacaaagtatcgtatc |
| | | gattttgatgttggttggttatgaattcaaactgtaagttggattgtga |
| | | gaagatcagaagttgaacgaacacatctttccgatcattcacctccaca |
| | | ctgcaacaacacggtacttcttccgcggcaggtctctgtcgccattctc |
| | | ttgtcctgttgttggctgtgagacgaggaaagcaacgacaagtttcaca |
| | | aaagggagttcctttaacgagatatgttttttataaagagtcccaatag |
| | | aaagacaaattgattcctccgtgcaaacgcgcaaataaacaccacgtcc |
| | | attatatccatatctttcagagtatccaacaagtgttgaaggacaggta |
| | | gttgaagtaacgtatcttccccctcgactggatccatcaacaaggcgaa |
| | | caaatccattcaacctctcataaattatctgatttaccaaaccgatATG |
| | | GCACAAGTCATCAACACCAACTCTCTCTCTCTCTTGACCCAGAACAACC |
| | | TCAACAAGTCCCAGTCCGCTCTCGGAACCGCTATCGAGCGTCTCTCTTC |
| | | CGGACTCCGTATCAACTCTGCTAAGGACGATGCTGCAGGACAGGCTATC |
| | | GCTAACCGTTTCACCGCTAACATCAAGGGACTCACTCAGGCTTCCCGTA |
| | | ACGCTAACGACGGAATCTCCATCGCTCAGACCACTGAGGGAGCTCTCAA |
| | | CGAGATCAACAACAACCTCCAGCGTGTGCGTGAGCTCGCTGTTCAGTCT |
| | | GCTAACTCTACCAACTCCCAGTCTGACCTCGACTCCATCCAGGCTGAGA |
| | | TCACCCAGCGTCTCAACGAGATCGACCGTGTGTCCGGACAGACTCAGTT |
| | | CAACGGAGTGAAGGTCCTCGCTCAGGACAACACCCTCACCATCCAGGTT |
| | | GGAGCCAACGACGGAGAGACTATCGATATCGATCTCAAGCAGATCAACT |
| | | CTCAGACCCTCGGACTCGATACCCTCAACGTGCAACAAAAGTACAAGGT |
| | | CTCTGATACCGCTGCAACTGTTACCGGATACGCCGATACTACCATCGCT |
| | | CTCGACAACAGTACTTTCAAGGCCTCTGCTACTGGACTCGGAGGAACTG |
| | | ACCAGAAGATCGATGGAGATCTCAAGTTCGATGATACCACTGGAAAGTA |
| | | CTACGCCAAGGTTACCGTTACCGGAGGAACTGGAAAGGATGGATACTAC |
| | | GAGGTTTCCGTTGATAAGACCAACGGAAAGGTGACTCTCGCTGGAGGAG |
| | | CTACTTCCCCACTCACCGGAGGACTCCCTGCTACCGCAACTGAGGATGT |
| | | GAAGAACGTGCAAGTTGCAAACGCTGATTTGACCGAGGCTAAGGCCGCA |
| | | TTGACCGCAGCAGGAGTTACCGGAACCGCATCTGTTGTTAAGATGTCTT |
| | | ACACTGATAACAACGGAAAGACTATCGATGGAGGACTCGCAGTTAAGGT |
| | | GGGAGATGATTACTACTCTGCAACTCAAAACAAGGATGGATCCATCAGT |
| | | ATCAACACTACCAAGTACACTGCAGATGACGGAACCTCCAAGACTGCAC |
| | | TCAACAAGCTCGGAGGAGCAGACGGAAAGACCGAGGTTGTTTCTATCGG |
| | | AGGAAAGACTTACGCTGCAAGTAAGGCCGAGGGACACAACTTCAAGGCA |
| | | CAGCCTGATCTCGCTGAGGCTGCTGCTACCACCACCGAGAACCCACTCC |
| | | AGAAGATCGATGCTGCTTTGGCACAGGTTGACACCCTCCGTTCTGACCT |
| | | CGGAGCTGTGCAGAACCGTTTCAACTCCGCTATCACCAACCTCGGAAAC |
| | | ACCGTGAACAACCTCACTTCTGCCCGTTCTCGTATCGAGGATTCCGACT |
| | | ACGCTACCGAGGTTTCCAACATGTCTCGTGCTCAGATCCTCCAGCAGGC |
| | | CGGAACCTCCGTTCTCGCTCAGGCTAACCAGGTTCCACAAAACGTCCTC |
| | | TCTCTCCTCCGTATGTGTTCTTCCTTGCCAGAGGAGTTGAACGCAAGTA |
| | | CCGAGCAAGTGGTTACTGATTACAAGGCATTCGCTGAGTCTCAGGGAGA |
| | | GTTGACCAACGACGTTCGTCTCGGAGGAATCATCGCAAAGGTCGATAAC |
| | | TTCAAGGACAAGACTCGTTTGGAGATCGTGAACCTCCCAATCAACAAGT |
| | | CTGGAAAGCCAGACATCGACCAAGAGCCTACCGGACGTTTCGCTGTGTA |
| | | CTTCGATGGATACTTGGAGCCTGTTGCCTTCTCCCAGGGACGTCTCGTT |
| | | ACTATCGTCGGAAAGGGAGCCGGAGAGGAGGAGGGAAAGATCGGAGAGC |
| | | ACGAGTACGTGTTCCCATTGGTTAAGGGACAAGGATACCGTCTCTGGAA |
| | | GATCGAGGAGCGTGTGCGTATGTACGACAGTCCAACCTACTTCTACCCT |
| | | TGTTACTCTATCAACTGCCGTATGCTCCGTAACGATTTCCCTCAAGACG |
| | | GAAAGGTCATCAAGCAGGTGAAGatggtgagcaagggcgaggagctgtt |
| | | caccggggtggtgcccatcctggtcgagctggacggcgacgtaaacggc |
| | | cacaagttcagcgtgtccggcgagggcgagggcgatgccacctacggca |
| | | agctgaccctgaagttcatctgcaccaccggcaagctgcccgtgccctg |
| | | gcccaccctcgtgaccaccctgacctacggcgtgcagtgcttcagccgc |
| | | taccccgaccacatgaagcagcacgacttcttcaagtccgccatgcccg |
| | | aaggctacgtccaggagcgcaccatcttcttcaaggacgacggcaacta |
| | | caagacccgcgccgaggtgaagttcgagggcgacaccctggtgaaccgc |
| | | atcgagctgaagggcatcgacttcaaggaggacggcaacatcctggggc |
| | | acaagctggagtacaactacaacagccacaacgtctatatcatggccga |
| | | caagcagaagaacggcatcaaggtgaacttcaagatccgccacaacatc |
| | | gaggacggcagcgtgcagctcgccgaccactaccagcagaacaccccca |
| | | tcggcgacggccccgtgctgctgcccgacaaccactacctgagcaccca |
| | | gtccgccctgagcaaagaccccaacgagaagcgcgatcacatggtcctg |
| | | ctggagttcgtgaccgccgccgggatcactctcggcatggacgagctgt |
| | | acaagtaagcggccgcatactggattggtgaatcaatgagccgtagcac |
| | | aatggttacattcggctagctaagatccaatggcaaggaccaagtgctg |
| | | gaacttgttttgctttagcagatcttagcgtgagaggtatttgtcctct |
| | | gtcaggagtagatagtagatgttctttttaaactaaaatgctaactgtt |
| | | ccgaattcctcatcgcagctaatccgtacatcaaaagacaaaatgctag |
| | | gtatgtgtactacatctcctgttgctagataagacatatgataggaaac |
| | | acaccatcaatagtcattgtagctttacttatactacgcatttgcactt |
| | | tcccctgagtggcagaggcgcattgagaaaatcgatctcaacatagttt |
| | | atgtagcatcccctagatccattactttaagtctccttcgtctttggtg |
| | | taggcatgttggacacaacgaggtaaaacacaacacaaacaatgtgtcc |
| | | agcaaagtagtagctgctccagttctccc |
| 17 | Flagellin + | gggggatccactagttctagagcggccggccgccaccgcggtggagctc |
| | Somatotropin | cagcttttgttccctttagtgagggttaattgcgcgcttggcgtaatca |
| | Italics: FCP | tggtcatagctgtttcctgtgtgaaattgttatccgctcacaattccac |
| | promoter and | acaacatacgagccggaagcataaagtgtaaagcctggggtgcctaatg |
| | terminator | agtgagctaactcacattaattgcgttgcgctcactgcccgctttccag |
| | CAPITAL | tcgggaaacctgtcgtgccagctgcattaatgaatcggccaacgcgcgg |
| | UNDERLINED | ggagaggcggtttgcgtattgggcgctcttccgcttcctcgctcactga |
| | Flagellin | ctcgctgcgctcggtcgttcggctgcggcgagcggtatcagctcactca |
| | Bold = Somatotropin | aaggcggtaatacggttatccacagaatcaggggataacgcaggaaaga |
| | Lower underlined = | acatgtgagcaaaaggccagcaaaaggccaggaaccgtaaaaaggccgc |
| | eGFP | gttgctggcgtttttccataggctccgcccccctgacgagcatcacaaa |
| | | aatcgacgctcaagtcagaggtggcgaaacccgacaggactataaagat |
| | | accaggcgtttccccctggaagctccctcgtgcgctctcctgttccgac |
| | | cctgccgcttaccggatacctgtccgcctttctcccttcgggaagcgtg |
| | | gcgctttctcatagctcacgctgtaggtatctcagttcggtgtaggtcg |
| | | ttcgctccaagctgggctgtgtgcacgaaccccccgttcagcccgaccg |
| | | ctgcgccttatccggtaactatcgtcttgagtccaacccggtaagacac |
| | | gacttatcgccactggcagcagccactggtaacaggattagcagagcga |
| | | ggtatgtaggcggtgctacagagttcttgaagtggtggcctaactacgg |
| | | ctacactagaaggacagtatttggtatctgcgctctgctgaagccagtt |
| | | accttcggaaaaagagttggtagctcttgatccggcaaacaaaccaccg |
| | | ctggtagcggtggtttttttgtttgcaagcagcagattacgcgcagaaa |
| | | aaaaggatctcaagaagatcctttgatcttttctacggggtctgacgct |
| | | cagtggaacgaaaactcacgttaagggattttggtcatgagattatcaa |
| | | aaaggatcttcacctagatccttttaaattaaaaatgaagttttaaatc |
| | | aatctaaagtatatatgagtaaacttggtctgacagttaccaatgctta |
| | | atcagtgaggcacctatctcagcgatctgtctatttcgttcatccatag |
| | | ttgcctgactccccgtcgtgtagataactacgatacgggagggcttacc |
| | | atctggccccagtgctgcaatgataccgcgagacccacgctcaccggct |
| | | ccagatttatcagcaataaaccagccagccggaagggccgagcgcagaa |
| | | gtggtcctgcaactttatccgcctccatccagtctattaattgttgccg |
| | | ggaagctagagtaagtagttcgccagttaatagtttgcgcaacgttgtt |
| | | gccattgctacaggcatcgtggtgtcacgctcgtcgtttggtatggctt |
| | | cattcagctccggttcccaacgatcaaggcgagttacatgatcccccat |
| | | gttgtgcaaaaaagcggttagctccttcggtcctccgatcgttgtcaga |
| | | agtaagttggccgcagtgttatcactcatggttatggcagcactgcata |
| | | attctcttactgtcatgccatccgtaagatgcttttctgtgactggtga |
| | | gtactcaaccaagtcattctgagaatagtgtatgcggcgaccgagttgc |
| | | tcttgcccggcgtcaatacgggataataccgcgccacatagcagaactt |
| | | taaaagtgctcatcattggaaaacgttcttcggggcgaaaactctcaag |
| | | gatcttaccgctgttgagatccagttcgatgtaacccactcgtgcaccc |
| | | aactgatcttcagcatcttttactttcaccagcgtttctgggtgagcaa |
| | | aaacaggaaggcaaaatgccgcaaaaaagggaataagggcgacacggaa |
| | | atgttgaatactcatactcttcctttttcaatattattgaagcatttat |
| | | cagggttattgtctcatgagcggatacatatttgaatgtatttagaaaa |
| | | ataaacaaataggggttccgcgcacatttccccgaaaagtgccacctaa |
| | | attgtaagcgttaatattttgttaaaattcgcgttaaatttttgttaaa |
| | | tcagctcattttttaaccaataggccgaaatcggcaaaatcccttataa |
| | | atcaaaagaatagaccgagatagggttgagtgttgttccagtttggaac |
| | | aagagtccactattaaagaacgtggactccaacgtcaaagggcgaaaaa |
| | | ccgtctatcagggcgatggcccactacgtgaaccatcaccctaatcaag |
| | | ttttttggggtcgaggtgccgtaaagcactaaatcggaaccctaaaggg |
| | | agcccccgatttagagcttgacggggaaagccggcgaacgtggcgagaa |
| | | aggaagggaagaaagcgaaaggagcgggcgctagggcgctggcaagtgt |
| | | agcggtcacgctgcgcgtaaccaccacacccgccgcgcttaatgcgccg |
| | | ctacagggcgcgtcccattcgccattcaggctgcgcaactgttgggaag |
| | | ggcgatcggtgcgggcctcttcgctattacgccagctggcgaaaggggg |
| | | atgtgctgcaaggcgattaagttgggtaacgccagggttttcccagtca |
| | | cgacgttgtaaaacgacggccagtgagcgcgcgtaatacgactcactat |
| | | agggcgaattgggtacgtaccgggccccccctcgaggtcgacggtatcg |
| | | ataagcttgcgctttttccgagaactccccataagtcaacggctccaat |
| | | caagaatgtatccgacaacggcgagcatagcaacacgtccgtctttgga |
| | | gtagaatcatcatgttgtggatgaatacacagatgaatgacattaaaag |
| | | catgaacatgttagagagtaggaggtagagattgatatggtagcattgc |
| | | gatgtttgtttttggtcagcatatgatgagtggataccaatatgatgaa |
| | | agttgaatctcgcgtttgagctcagcggtacgttattgatcgaaagtag |
| | | cctgatcaaaatccttggagagtacaagaggatcaaagaatccagtggg |
| | | ggcgataactccaagctcgttctcaaagaggcaatggaggtagaaactc |
| | | atcccagttgagaagaagtgaaggcagtggcggtggcgaaagcagaggc |
| | | aacgaggacagacttcctgtgggttgatgcaacgaatatttccagaagg |
| | | agaagtttagagagttgaaccgctacctacaatgacaaagtatcgtatc |
| | | gattttgatgttggttggttatgaattcaaactgtaagttggattgtga |
| | | gaagatcagaagttgaacgaacacatctttccgatcattcacctccaca |
| | | ctgcaacaacacggtacttcttccgcggcaggtctctgtcgccattctc |
| | | ttgtcctgttgttggctgtgagacgaggaaagcaacgacaagtttcaca |
| | | aaagggagttcctttaacgagatatgttttttataaagagtcccaatag |
| | | aaagacaaattgattcctccgtgcaaacgcgcaaataaacaccacgtcc |
| | | attatatccatatctttcagagtatccaacaagtgttgaaggacaggta |
| | | gttgaagtaacgtatcttccccctcgactggatccatcaacaaggcgaa |
| | | caaatccattcaacctctcataaattatctgatttaccaaaccgatATG |
| | | GCACAAGTCATCAACACCAACTCTCTCTCTCTCTTGACCCAGAACAACC |
| | | TCAACAAGTCCCAGTCCGCTCTCGGAACCGCTATCGAGCGTCTCTCTTC |
| | | CGGACTCCGTATCAACTCTGCTAAGGACGATGCTGCAGGACAGGCTATC |
| | | GCTAACCGTTTCACCGCTAACATCAAGGGACTCACTCAGGCTTCCCGTA |
| | | ACGCTAACGACGGAATCTCCATCGCTCAGACCACTGAGGGAGCTCTCAA |
| | | CGAGATCAACAACAACCTCCAGCGTGTGCGTGAGCTCGCTGTTCAGTCT |
| | | GCTAACTCTACCAACTCCCAGTCTGACCTCGACTCCATCCAGGCTGAGA |
| | | TCACCCAGCGTCTCAACGAGATCGACCGTGTGTCCGGACAGACTCAGTT |
| | | CAACGGAGTGAAGGTCCTCGCTCAGGACAACACCCTCACCATCCAGGTT |
| | | GGAGCCAACGACGGAGAGACTATCGATATCGATCTCAAGCAGATCAACT |
| | | CTCAGACCCTCGGACTCGATACCCTCAACGTGCAACAAAAGTACAAGGT |
| | | CTCTGATACCGCTGCAACTGTTACCGGATACGCCGATACTACCATCGCT |
| | | CTCGACAACAGTACTTTCAAGGCCTCTGCTACTGGACTCGGAGGAACTG |
| | | ACCAGAAGATCGATGGAGATCTCAAGTTCGATGATACCACTGGAAAGTA |
| | | CTACGCCAAGGTTACCGTTACCGGAGGAACTGGAAAGGATGGATACTAC |
| | | GAGGTTTCCGTTGATAAGACCAACGGAAAGGTGACTCTCGCTGGAGGAG |
| | | CTACTTCCCCACTCACCGGAGGACTCCCTGCTACCGCAACTGAGGATGT |
| | | GAAGAACGTGCAAGTTGCAAACGCTGATTTGACCGAGGCTAAGGCCGCA |
| | | TTGACCGCAGCAGGAGTTACCGGAACCGCATCTGTTGTTAAGATGTCTT |
| | | ACACTGATAACAACGGAAAGACTATCGATGGAGGACTCGCAGTTAAGGT |
| | | GGGAGATGATTACTACTCTGCAACTCAAAACAAGGATGGATCCATCAGT |
| | | ATCAACACTACCAAGTACACTGCAGATGACGGAACCTCCAAGACTGCAC |
| | | TCAACAAGCTCGGAGGAGCAGACGGAAAGACCGAGGTTGTTTCTATCGG |
| | | AGGAAAGACTTACGCTGCAAGTAAGGCCGAGGGACACAACTTCAAGGCA |
| | | CAGCCTGATCTCGCTGAGGCTGCTGCTACCACCACCGAGAACCCACTCC |
| | | AGAAGATCGATGCTGCTTTGGCACAGGTTGACACCCTCCGTTCTGACCT |
| | | CGGAGCTGTGCAGAACCGTTTCAACTCCGCTATCACCAACCTCGGAAAC |
| | | ACCGTGAACAACCTCACTTCTGCCCGTTCTCGTATCGAGGATTCCGACT |
| | | ACGCTACCGAGGTTTCCAACATGTCTCGTGCTCAGATCCTCCAGCAGGC |
| | | CGGAACCTCCGTTCTCGCTCAGGCTAACCAGGTTCCACAAAACGTCCTC |
| | | TCTCTCCTCCGTATGCAACCAATCACCGAGAACCAGCGTTTGTTCTCCA |
| | | TCGCTGTCGGACGTGTGCAATACTTGCACCTCGTCGCCAAGAAGCTCTT |
| | | CTCCGATTTCGAGAACAGTTTGCAATTGGAGGACCAGCGTTTGCTCAAC |
| | | AAGATCGCATCTAAGGAGTTCTGCCACTCTGATAACTTCTTGTCCCCTA |
| | | TCGACAAGCATGAGACTCAAGGAAGTTCTGTCCAGAAGTTGCTCTCTGT |
| | | GTCCTACCGTTTGATCGAGAGTTGGGAGTTCTTCTCTCGTTTCCTCGTG |
| | | GCTTCTTTCGCCGTTCGTACCCAGGTCACTTCTAAGCTCTCTGAGTTGA |
| | | AGATGGGATTGCTCAAGTTGATCGAGGCAAACCAAGATGGAGCTGGAGG |
| | | ATTCTCCGAGTCCAGTGTTTTGCAGCTCACCCCATACGGAAACAGTGAG |
| | | TTGTTCGCCTGTTTCAAGAAGGACATGCATAAGGTTGAGACTTACTTGA |
| | | CTGTCGCCAAGTGTCGTCTCTTCCCTGAGGCAAACTGCACTCTCatggt |
| | | gagcaagggcgaggagctgttcaccggggtggtgcccatcctggtcgag |
| | | ctggacggcgacgtaaacggccacaagttcagcgtgtccggcgagggcg |
| | | agggcgatgccacctacggcaagctgaccctgaagttcatctgcaccac |
| | | cggcaagctgcccgtgccctggcccaccctcgtgaccaccctgacctac |
| | | ggcgtgcagtgcttcagccgctaccccgaccacatgaagcagcacgact |
| | | tcttcaagtccgccatgcccgaaggctacgtccaggagcgcaccatctt |
| | | cttcaaggacgacggcaactacaagacccgcgccgaggtgaagttcgag |
| | | ggcgacaccctggtgaaccgcatcgagctgaagggcatcgacttcaagg |
| | | aggacggcaacatcctggggcacaagctggagtacaactacaacagcca |
| | | caacgtctatatcatggccgacaagcagaagaacggcatcaaggtgaac |
| | | ttcaagatccgccacaacatcgaggacggcagcgtgcagctcgccgacc |
| | | actaccagcagaacacccccatcggcgacggccccgtgctgctgcccga |
| | | caaccactacctgagcacccagtccgccctgagcaaagaccccaacgag |
| | | aagcgcgatcacatggtcctgctggagttcgtgaccgccgccgggatca |
| | | ctctcggcatggacgagctgtacaagtaagcggccgcatactggattgg |
| | | tgaatcaatgagccgtagcacaatggttacattcggctagctaagatcc |
| | | aatggcaaggaccaagtgctggaacttgttttgctttagcagatcttag |
| | | cgtgagaggtatttgtcctctgtcaggagtagatagtagatgttctttt |
| | | taaactaaaatgctaactgttccgaattcctcatcgcagctaatccgta |
| | | catcaaaagacaaaatgctaggtatgtgtactacatctcctgttgctag |
| | | ataagacatatgataggaaacacaccatcaatagtcattgtagctttac |
| | | ttatactacgcatttgcactttcccctgagtggcagaggcgcattgaga |
| | | aaatcgatctcaacatagtttatgtagcatcccctagatccattacttt |
| | | aagtctccttcgtctttggtgtaggcatgttggacacaacgaggtaaaa |
| | | cacaacacaaacaatgtgtccagcaaagtagtagctgctccagttctcc |
| | | c |
| 18 | Flagellin + Vibrio | gggggatccactagttctagagcggccggccgccaccgcggtggagctc |
| | parahaemolyticus | cagcttttgttccctttagtgagggttaattgcgcgcttggcgtaatca |
| | pirA^{VP} | tggtcatagctgtttcctgtgtgaaattgttatccgctcacaattccac |
| | Italics: FCP | acaacatacgagccggaagcataaagtgtaaagcctggggtgcctaatg |
| | promoter and | agtgagctaactcacattaattgcgttgcgctcactgcccgctttccag |
| | terminator | tcgggaaacctgtcgtgccagctgcattaatgaatcggccaacgcgcgg |
| | CAPITAL | ggagaggcggtttgcgtattgggcgctcttccgcttcctcgctcactga |
| | UNDERLINED = | ctcgctgcgctcggtcgttcggctgcggcgagcggtatcagctcactca |
| | Flagellin | aaggcggtaatacggttatccacagaatcaggggataacgcaggaaaga |
| | Bold = pirA^{VP} | acatgtgagcaaaaggccagcaaaaggccaggaaccgtaaaaaggccgc |
| | Lower underlined = | gttgctggcgtttttccataggctccgcccccctgacgagcatcacaaa |
| | eGFP | aatcgacgctcaagtcagaggtggcgaaacccgacaggactataaagat |
| | | accaggcgtttccccctggaagctccctcgtgcgctctcctgttccgac |
| | | cctgccgcttaccggatacctgtccgcctttctcccttcgggaagcgtg |
| | | gcgctttctcatagctcacgctgtaggtatctcagttcggtgtaggtcg |
| | | ttcgctccaagctgggctgtgtgcacgaaccccccgttcagcccgaccg |
| | | ctgcgccttatccggtaactatcgtcttgagtccaacccggtaagacac |
| | | gacttatcgccactggcagcagccactggtaacaggattagcagagcga |
| | | ggtatgtaggcggtgctacagagttcttgaagtggtggcctaactacgg |
| | | ctacactagaaggacagtatttggtatctgcgctctgctgaagccagtt |
| | | accttcggaaaaagagttggtagctcttgatccggcaaacaaaccaccg |
| | | ctggtagcggtggtttttttgtttgcaagcagcagattacgcgcagaaa |
| | | aaaaggatctcaagaagatcctttgatcttttctacggggtctgacgct |
| | | cagtggaacgaaaactcacgttaagggattttggtcatgagattatcaa |
| | | aaaggatcttcacctagatccttttaaattaaaaatgaagttttaaatc |
| | | aatctaaagtatatatgagtaaacttggtctgacagttaccaatgctta |
| | | atcagtgaggcacctatctcagcgatctgtctatttcgttcatccatag |
| | | ttgcctgactccccgtcgtgtagataactacgatacgggagggcttacc |
| | | atctggccccagtgctgcaatgataccgcgagacccacgctcaccggct |
| | | ccagatttatcagcaataaaccagccagccggaagggccgagcgcagaa |
| | | gtggtcctgcaactttatccgcctccatccagtctattaattgttgccg |
| | | ggaagctagagtaagtagttcgccagttaatagtttgcgcaacgttgtt |
| | | gccattgctacaggcatcgtggtgtcacgctcgtcgtttggtatggctt |
| | | cattcagctccggttcccaacgatcaaggcgagttacatgatcccccat |
| | | gttgtgcaaaaaagcggttagctccttcggtcctccgatcgttgtcaga |
| | | agtaagttggccgcagtgttatcactcatggttatggcagcactgcata |
| | | attctcttactgtcatgccatccgtaagatgcttttctgtgactggtga |
| | | gtactcaaccaagtcattctgagaatagtgtatgcggcgaccgagttgc |
| | | tcttgcccggcgtcaatacgggataataccgcgccacatagcagaactt |
| | | taaaagtgctcatcattggaaaacgttcttcggggcgaaaactctcaag |
| | | gatcttaccgctgttgagatccagttcgatgtaacccactcgtgcaccc |
| | | aactgatcttcagcatcttttactttcaccagcgtttctgggtgagcaa |
| | | aaacaggaaggcaaaatgccgcaaaaaagggaataagggcgacacggaa |
| | | atgttgaatactcatactcttcctttttcaatattattgaagcatttat |
| | | cagggttattgtctcatgagcggatacatatttgaatgtatttagaaaa |
| | | ataaacaaataggggttccgcgcacatttccccgaaaagtgccacctaa |
| | | attgtaagcgttaatattttgttaaaattcgcgttaaatttttgttaaa |
| | | tcagctcattttttaaccaataggccgaaatcggcaaaatcccttataa |
| | | atcaaaagaatagaccgagatagggttgagtgttgttccagtttggaac |
| | | aagagtccactattaaagaacgtggactccaacgtcaaagggcgaaaaa |
| | | ccgtctatcagggcgatggcccactacgtgaaccatcaccctaatcaag |
| | | ttttttggggtcgaggtgccgtaaagcactaaatcggaaccctaaaggg |
| | | agcccccgatttagagcttgacggggaaagccggcgaacgtggcgagaa |
| | | aggaagggaagaaagcgaaaggagcgggcgctagggcgctggcaagtgt |
| | | agcggtcacgctgcgcgtaaccaccacacccgccgcgcttaatgcgccg |
| | | ctacagggcgcgtcccattcgccattcaggctgcgcaactgttgggaag |
| | | ggcgatcggtgcgggcctcttcgctattacgccagctggcgaaaggggg |
| | | atgtgctgcaaggcgattaagttgggtaacgccagggttttcccagtca |
| | | cgacgttgtaaaacgacggccagtgagcgcgcgtaatacgactcactat |
| | | agggcgaattgggtacgtaccgggccccccctcgaggtcgacggtatcg |
| | | ataagcttgcgctttttccgagaactccccataagtcaacggctccaat |
| | | caagaatgtatccgacaacggcgagcatagcaacacgtccgtctttgga |
| | | gtagaatcatcatgttgtggatgaatacacagatgaatgacattaaaag |
| | | catgaacatgttagagagtaggaggtagagattgatatggtagcattgc |
| | | gatgtttgtttttggtcagcatatgatgagtggataccaatatgatgaa |
| | | agttgaatctcgcgtttgagctcagcggtacgttattgatcgaaagtag |
| | | cctgatcaaaatccttggagagtacaagaggatcaaagaatccagtggg |
| | | ggcgataactccaagctcgttctcaaagaggcaatggaggtagaaactc |
| | | atcccagttgagaagaagtgaaggcagtggcggtggcgaaagcagaggc |
| | | aacgaggacagacttcctgtgggttgatgcaacgaatatttccagaagg |
| | | agaagtttagagagttgaaccgctacctacaatgacaaagtatcgtatc |
| | | gattttgatgttggttggttatgaattcaaactgtaagttggattgtga |
| | | gaagatcagaagttgaacgaacacatctttccgatcattcacctccaca |
| | | ctgcaacaacacggtacttcttccgcggcaggtctctgtcgccattctc |
| | | ttgtcctgttgttggctgtgagacgaggaaagcaacgacaagtttcaca |
| | | aaagggagttcctttaacgagatatgttttttataaagagtcccaatag |
| | | aaagacaaattgattcctccgtgcaaacgcgcaaataaacaccacgtcc |
| | | attatatccatatctttcagagtatccaacaagtgttgaaggacaggta |
| | | gttgaagtaacgtatcttccccctcgactggatccatcaacaaggcgaa |
| | | caaatccattcaacctctcataaattatctgatttaccaaaccgatATG |
| | | GCACAAGTCATCAACACCAACTCTCTCTCTCTCTTGACCCAGAACAACC |
| | | TCAACAAGTCCCAGTCCGCTCTCGGAACCGCTATCGAGCGTCTCTCTTC |
| | | CGGACTCCGTATCAACTCTGCTAAGGACGATGCTGCAGGACAGGCTATC |
| | | GCTAACCGTTTCACCGCTAACATCAAGGGACTCACTCAGGCTTCCCGTA |
| | | ACGCTAACGACGGAATCTCCATCGCTCAGACCACTGAGGGAGCTCTCAA |
| | | CGAGATCAACAACAACCTCCAGCGTGTGCGTGAGCTCGCTGTTCAGTCT |
| | | GCTAACTCTACCAACTCCCAGTCTGACCTCGACTCCATCCAGGCTGAGA |
| | | TCACCCAGCGTCTCAACGAGATCGACCGTGTGTCCGGACAGACTCAGTT |
| | | CAACGGAGTGAAGGTCCTCGCTCAGGACAACACCCTCACCATCCAGGTT |
| | | GGAGCCAACGACGGAGAGACTATCGATATCGATCTCAAGCAGATCAACT |
| | | CTCAGACCCTCGGACTCGATACCCTCAACGTGCAACAAAAGTACAAGGT |
| | | CTCTGATACCGCTGCAACTGTTACCGGATACGCCGATACTACCATCGCT |
| | | CTCGACAACAGTACTTTCAAGGCCTCTGCTACTGGACTCGGAGGAACTG |
| | | ACCAGAAGATCGATGGAGATCTCAAGTTCGATGATACCACTGGAAAGTA |
| | | CTACGCCAAGGTTACCGTTACCGGAGGAACTGGAAAGGATGGATACTAC |
| | | GAGGTTTCCGTTGATAAGACCAACGGAAAGGTGACTCTCGCTGGAGGAG |
| | | CTACTTCCCCACTCACCGGAGGACTCCCTGCTACCGCAACTGAGGATGT |
| | | GAAGAACGTGCAAGTTGCAAACGCTGATTTGACCGAGGCTAAGGCCGCA |
| | | TTGACCGCAGCAGGAGTTACCGGAACCGCATCTGTTGTTAAGATGTCTT |
| | | ACACTGATAACAACGGAAAGACTATCGATGGAGGACTCGCAGTTAAGGT |
| | | GGGAGATGATTACTACTCTGCAACTCAAAACAAGGATGGATCCATCAGT |
| | | ATCAACACTACCAAGTACACTGCAGATGACGGAACCTCCAAGACTGCAC |
| | | TCAACAAGCTCGGAGGAGCAGACGGAAAGACCGAGGTTGTTTCTATCGG |
| | | AGGAAAGACTTACGCTGCAAGTAAGGCCGAGGGACACAACTTCAAGGCA |
| | | CAGCCTGATCTCGCTGAGGCTGCTGCTACCACCACCGAGAACCCACTCC |
| | | AGAAGATCGATGCTGCTTTGGCACAGGTTGACACCCTCCGTTCTGACCT |
| | | CGGAGCTGTGCAGAACCGTTTCAACTCCGCTATCACCAACCTCGGAAAC |
| | | ACCGTGAACAACCTCACTTCTGCCCGTTCTCGTATCGAGGATTCCGACT |
| | | ACGCTACCGAGGTTTCCAACATGTCTCGTGCTCAGATCCTCCAGCAGGC |
| | | CGGAACCTCCGTTCTCGCTCAGGCTAACCAGGTTCCACAAAACGTCCTC |
| | | TCTCTCCTCCGTATGTCTAACAACATCAAGCATGAGACTGATTACTCTC |
| | | ACGATTGGACCGTCGAGCCAAACGGAGGAGTGACCGAGGTGGATTCTAA |
| | | GCATACCCCAATCATCCCTGAGGTGGGACGTTCTGTCGATATCGAGAAC |
| | | ACTGGACGTGGAGAGTTGACCATCCAATACCAATGGGGAGCCCCATTCA |
| | | TGGCTGGAGGATGGAAGGTTGCCAAGTCTCATGTCGTGCAACGTGATGA |
| | | GACCTACCATTTGCAACGTCCTGATAACGCTTTCTACCACCAACGTATC |
| | | GTTGTGATCAACAACGGAGCCTCTCGTGGATTCTGTACCATCTACTACC |
| | | ATatggtgagcaagggcgaggagctgttcaccggggtggtgcccatcct |
| | | ggtcgagctggacggcgacgtaaacggccacaagttcagcgtgtccggc |
| | | gagggcgagggcgatgccacctacggcaagctgaccctgaagttcatct |
| | | gcaccaccggcaagctgcccgtgccctggcccaccctcgtgaccaccct |
| | | gacctacggcgtgcagtgcttcagccgctaccccgaccacatgaagcag |
| | | cacgacttcttcaagtccgccatgcccgaaggctacgtccaggagcgca |
| | | ccatcttcttcaaggacgacggcaactacaagacccgcgccgaggtgaa |
| | | gttcgagggcgacaccctggtgaaccgcatcgagctgaagggcatcgac |
| | | ttcaaggaggacggcaacatcctggggcacaagctggagtacaactaca |
| | | acagccacaacgtctatatcatggccgacaagcagaagaacggcatcaa |
| | | ggtgaacttcaagatccgccacaacatcgaggacggcagcgtgcagctc |
| | | gccgaccactaccagcagaacacccccatcggcgacggccccgtgctgc |
| | | tgcccgacaaccactacctgagcacccagtccgccctgagcaaagaccc |
| | | caacgagaagcgcgatcacatggtcctgctggagttcgtgaccgccgcc |
| | | gggatcactctcggcatggacgagctgtacaagtaagcggccgcatact |
| | | ggattggtgaatcaatgagccgtagcacaatggttacattcggctagct |
| | | aagatccaatggcaaggaccaagtgctggaacttgttttgctttagcag |
| | | atcttagcgtgagaggtatttgtcctctgtcaggagtagatagtagatg |
| | | ttctttttaaactaaaatgctaactgttccgaattcctcatcgcagcta |
| | | atccgtacatcaaaagacaaaatgctaggtatgtgtactacatctcctg |
| | | ttgctagataagacatatgataggaaacacaccatcaatagtcattgta |
| | | gctttacttatactacgcatttgcactttcccctgagtggcagaggcgc |
| | | attgagaaaatcgatctcaacatagtttatgtagcatcccctagatcca |
| | | ttactttaagtctccttcgtctttggtgtaggcatgttggacacaacga |
| | | ggtaaaacacaacacaaacaatgtgtccagcaaagtagtagctgctcca |
| | | gttctccc |
| 19 | Flagellin + Vibrio | gggggatccactagttctagagcggccggccgccaccgcggtggagctc |
| | parahaemolyticus | cagcttttgttccctttagtgagggttaattgcgcgcttggcgtaatca |
| | pirB^{VP} | tggtcatagctgtttcctgtgtgaaattgttatccgctcacaattccac |
| | Italics: FCP | acaacatacgagccggaagcataaagtgtaaagcctggggtgcctaatg |
| | promoter and | agtgagctaactcacattaattgcgttgcgctcactgcccgctttccag |
| | terminator | tcgggaaacctgtcgtgccagctgcattaatgaatcggccaacgcgcgg |
| | CAPITAL | ggagaggcggtttgcgtattgggcgctcttccgcttcctcgctcactga |
| | UNDERLINED | ctcgctgcgctcggtcgttcggctgcggcgagcggtatcagctcactca |
| | Flagellin | aaggcggtaatacggttatccacagaatcaggggataacgcaggaaaga |
| | Bold = pirB^{VP} | acatgtgagcaaaaggccagcaaaaggccaggaaccgtaaaaaggccgc |
| | Lower underlined = | gttgctggcgtttttccataggctccgcccccctgacgagcatcacaaa |
| | eGFP | aatcgacgctcaagtcagaggtggcgaaacccgacaggactataaagat |
| | | accaggcgtttccccctggaagctccctcgtgcgctctcctgttccgac |
| | | cctgccgcttaccggatacctgtccgcctttctcccttcgggaagcgtg |
| | | gcgctttctcatagctcacgctgtaggtatctcagttcggtgtaggtcg |
| | | ttcgctccaagctgggctgtgtgcacgaaccccccgttcagcccgaccg |
| | | ctgcgccttatccggtaactatcgtcttgagtccaacccggtaagacac |
| | | gacttatcgccactggcagcagccactggtaacaggattagcagagcga |
| | | ggtatgtaggcggtgctacagagttcttgaagtggtggcctaactacgg |
| | | ctacactagaaggacagtatttggtatctgcgctctgctgaagccagtt |
| | | accttcggaaaaagagttggtagctcttgatccggcaaacaaaccaccg |
| | | ctggtagcggtggtttttttgtttgcaagcagcagattacgcgcagaaa |
| | | aaaaggatctcaagaagatcctttgatcttttctacggggtctgacgct |
| | | cagtggaacgaaaactcacgttaagggattttggtcatgagattatcaa |
| | | aaaggatcttcacctagatccttttaaattaaaaatgaagttttaaatc |
| | | aatctaaagtatatatgagtaaacttggtctgacagttaccaatgctta |
| | | atcagtgaggcacctatctcagcgatctgtctatttcgttcatccatag |
| | | ttgcctgactccccgtcgtgtagataactacgatacgggagggcttacc |
| | | atctggccccagtgctgcaatgataccgcgagacccacgctcaccggct |
| | | ccagatttatcagcaataaaccagccagccggaagggccgagcgcagaa |
| | | gtggtcctgcaactttatccgcctccatccagtctattaattgttgccg |
| | | ggaagctagagtaagtagttcgccagttaatagtttgcgcaacgttgtt |
| | | gccattgctacaggcatcgtggtgtcacgctcgtcgtttggtatggctt |
| | | cattcagctccggttcccaacgatcaaggcgagttacatgatcccccat |
| | | gttgtgcaaaaaagcggttagctccttcggtcctccgatcgttgtcaga |
| | | agtaagttggccgcagtgttatcactcatggttatggcagcactgcata |
| | | attctcttactgtcatgccatccgtaagatgcttttctgtgactggtga |
| | | gtactcaaccaagtcattctgagaatagtgtatgcggcgaccgagttgc |
| | | tcttgcccggcgtcaatacgggataataccgcgccacatagcagaactt |
| | | taaaagtgctcatcattggaaaacgttcttcggggcgaaaactctcaag |
| | | gatcttaccgctgttgagatccagttcgatgtaacccactcgtgcaccc |
| | | aactgatcttcagcatcttttactttcaccagcgtttctgggtgagcaa |
| | | aaacaggaaggcaaaatgccgcaaaaaagggaataagggcgacacggaa |
| | | atgttgaatactcatactcttcctttttcaatattattgaagcatttat |
| | | cagggttattgtctcatgagcggatacatatttgaatgtatttagaaaa |
| | | ataaacaaataggggttccgcgcacatttccccgaaaagtgccacctaa |
| | | attgtaagcgttaatattttgttaaaattcgcgttaaatttttgttaaa |
| | | tcagctcattttttaaccaataggccgaaatcggcaaaatcccttataa |
| | | atcaaaagaatagaccgagatagggttgagtgttgttccagtttggaac |
| | | aagagtccactattaaagaacgtggactccaacgtcaaagggcgaaaaa |
| | | ccgtctatcagggcgatggcccactacgtgaaccatcaccctaatcaag |
| | | ttttttggggtcgaggtgccgtaaagcactaaatcggaaccctaaaggg |
| | | agcccccgatttagagcttgacggggaaagccggcgaacgtggcgagaa |
| | | aggaagggaagaaagcgaaaggagcgggcgctagggcgctggcaagtgt |
| | | agcggtcacgctgcgcgtaaccaccacacccgccgcgcttaatgcgccg |
| | | ctacagggcgcgtcccattcgccattcaggctgcgcaactgttgggaag |
| | | ggcgatcggtgcgggcctcttcgctattacgccagctggcgaaaggggg |
| | | atgtgctgcaaggcgattaagttgggtaacgccagggttttcccagtca |
| | | cgacgttgtaaaacgacggccagtgagcgcgcgtaatacgactcactat |
| | | agggcgaattgggtacgtaccgggccccccctcgaggtcgacggtatcg |
| | | ataagcttgcgctttttccgagaactccccataagtcaacggctccaat |
| | | caagaatgtatccgacaacggcgagcatagcaacacgtccgtctttgga |
| | | gtagaatcatcatgttgtggatgaatacacagatgaatgacattaaaag |
| | | catgaacatgttagagagtaggaggtagagattgatatggtagcattgc |
| | | gatgtttgtttttggtcagcatatgatgagtggataccaatatgatgaa |
| | | agttgaatctcgcgtttgagctcagcggtacgttattgatcgaaagtag |
| | | cctgatcaaaatccttggagagtacaagaggatcaaagaatccagtggg |
| | | ggcgataactccaagctcgttctcaaagaggcaatggaggtagaaactc |
| | | atcccagttgagaagaagtgaaggcagtggcggtggcgaaagcagaggc |
| | | aacgaggacagacttcctgtgggttgatgcaacgaatatttccagaagg |
| | | agaagtttagagagttgaaccgctacctacaatgacaaagtatcgtatc |
| | | gattttgatgttggttggttatgaattcaaactgtaagttggattgtga |
| | | gaagatcagaagttgaacgaacacatctttccgatcattcacctccaca |
| | | ctgcaacaacacggtacttcttccgcggcaggtctctgtcgccattctc |
| | | ttgtcctgttgttggctgtgagacgaggaaagcaacgacaagtttcaca |
| | | aaagggagttcctttaacgagatatgttttttataaagagtcccaatag |
| | | aaagacaaattgattcctccgtgcaaacgcgcaaataaacaccacgtcc |
| | | attatatccatatctttcagagtatccaacaagtgttgaaggacaggta |
| | | gttgaagtaacgtatcttccccctcgactggatccatcaacaaggcgaa |
| | | caaatccattcaacctctcataaattatctgatttaccaaaccgatATG |
| | | GCACAAGTCATCAACACCAACTCTCTCTCTCTCTTGACCCAGAACAACC |
| | | TCAACAAGTCCCAGTCCGCTCTCGGAACCGCTATCGAGCGTCTCTCTTC |
| | | CGGACTCCGTATCAACTCTGCTAAGGACGATGCTGCAGGACAGGCTATC |
| | | GCTAACCGTTTCACCGCTAACATCAAGGGACTCACTCAGGCTTCCCGTA |
| | | ACGCTAACGACGGAATCTCCATCGCTCAGACCACTGAGGGAGCTCTCAA |
| | | CGAGATCAACAACAACCTCCAGCGTGTGCGTGAGCTCGCTGTTCAGTCT |
| | | GCTAACTCTACCAACTCCCAGTCTGACCTCGACTCCATCCAGGCTGAGA |
| | | TCACCCAGCGTCTCAACGAGATCGACCGTGTGTCCGGACAGACTCAGTT |
| | | CAACGGAGTGAAGGTCCTCGCTCAGGACAACACCCTCACCATCCAGGTT |
| | | GGAGCCAACGACGGAGAGACTATCGATATCGATCTCAAGCAGATCAACT |
| | | CTCAGACCCTCGGACTCGATACCCTCAACGTGCAACAAAAGTACAAGGT |
| | | CTCTGATACCGCTGCAACTGTTACCGGATACGCCGATACTACCATCGCT |
| | | CTCGACAACAGTACTTTCAAGGCCTCTGCTACTGGACTCGGAGGAACTG |
| | | ACCAGAAGATCGATGGAGATCTCAAGTTCGATGATACCACTGGAAAGTA |
| | | CTACGCCAAGGTTACCGTTACCGGAGGAACTGGAAAGGATGGATACTAC |
| | | GAGGTTTCCGTTGATAAGACCAACGGAAAGGTGACTCTCGCTGGAGGAG |
| | | CTACTTCCCCACTCACCGGAGGACTCCCTGCTACCGCAACTGAGGATGT |
| | | GAAGAACGTGCAAGTTGCAAACGCTGATTTGACCGAGGCTAAGGCCGCA |
| | | TTGACCGCAGCAGGAGTTACCGGAACCGCATCTGTTGTTAAGATGTCTT |
| | | ACACTGATAACAACGGAAAGACTATCGATGGAGGACTCGCAGTTAAGGT |
| | | GGGAGATGATTACTACTCTGCAACTCAAAACAAGGATGGATCCATCAGT |
| | | ATCAACACTACCAAGTACACTGCAGATGACGGAACCTCCAAGACTGCAC |
| | | TCAACAAGCTCGGAGGAGCAGACGGAAAGACCGAGGTTGTTTCTATCGG |
| | | AGGAAAGACTTACGCTGCAAGTAAGGCCGAGGGACACAACTTCAAGGCA |
| | | CAGCCTGATCTCGCTGAGGCTGCTGCTACCACCACCGAGAACCCACTCC |
| | | AGAAGATCGATGCTGCTTTGGCACAGGTTGACACCCTCCGTTCTGACCT |
| | | CGGAGCTGTGCAGAACCGTTTCAACTCCGCTATCACCAACCTCGGAAAC |
| | | ACCGTGAACAACCTCACTTCTGCCCGTTCTCGTATCGAGGATTCCGACT |
| | | ACGCTACCGAGGTTTCCAACATGTCTCGTGCTCAGATCCTCCAGCAGGC |
| | | CGGAACCTCCGTTCTCGCTCAGGCTAACCAGGTTCCACAAAACGTCCTC |
| | | TCTCTCCTCCGTATGACTAACGAGTACGTTGTGACTATGTCTTCTCTCA |
| | | CTGAGTTCAACCCAAACAACGCTCGTAAGTCTTACCTCTTCGATAACTA |
| | | CGAGGTTGATCCTAACTACGCCTTCAAGGCCATGGTTTCTTTCGGATTG |
| | | TCTAACATCCCATACGCCGGAGGATTCCTCTCTACTTTGTGGAACATCT |
| | | TCTGGCCAAACACTCCAAACGAGCCTGATATCGAGAACATCTGGGAGCA |
| | | ATTGCGTGATCGTATCCAAGATCTCGTTGATGAGTCTATCATCGATGCC |
| | | ATCAACGGAATCCTCGATTCTAAGATCAAGGAGACTCGTGATAAGATCC |
| | | AAGATATCAACGAGACCATCGAGAACTTCGGATACGCTGCCGCTAAGGA |
| | | TGATTACATCGGACTCGTGACTCACTACTTGATCGGATTGGAGGAGAAC |
| | | TTCAAGCGTGAGCTCGATGGAGATGAGTGGTTGGGATACGCTATCTTGC |
| | | CTCTCTTGGCCACCACTGTCTCTTTGCAAATCACCTACATGGCTTGTGG |
| | | ATTGGATTACAAGGATGAGTTCGGATTCACTGATTCTGATGTCCATAAG |
| | | CTCACCCGTAACATCGATAAGTTGTACGATGATGTTTCTTCTTACATCA |
| | | CTGAGTTGGCCGCTTGGGCCGATAACGATTCTTACAACAACGCCAACCA |
| | | AGATAACGTTTACGATGAGGTTATGGGAGCCCGTTCTTGGTGTACCGTG |
| | | CATGGATTCGAGCATATGTTGATCTGGCAAAAGATCAAGGAGTTGAAGA |
| | | AGGTGGATGTGTTCGTGCACTCTAACCTCATCTCTTACTCTCCAGCTGT |
| | | GGGATTCCCATCTGGAAACTTCAACTACATCGCTACTGGAACCGAGGAT |
| | | GAGATCCCTCAACCACTCAAGCCAAACATGTTCGGAGAGCGTCGTAACC |
| | | GTATCGTGAAGATCGAGTCTTGGAACTCTATCGAGATCCACTACTACAA |
| | | CCGTGTTGGACGTTTGAAGCTCACCTACGAGAACGGAGAGGTGGTCGAG |
| | | TTGGGAAAGGCCCATAAGTACGATGAGCATTACCAATCTATCGAGCTCA |
| | | ACGGAGCCTACATCAAGTACGTTGATGTGATCGCCAACGGACCTGAGGC |
| | | TATCGATCGTATCGTCTTCCACTTCTCTGATGATCGTACCTTCGTGGTT |
| | | GGAGAGAACTCTGGAAAGCCATCTGTGCGTCTCCAACTCGAGGGACACT |
| | | TCATCTGTGGAATGTTGGCTGATCAAGAGGGATCTGATAAGGTTGCCGC |
| | | TTTCTCTGTCGCTTACGAGCTCTTCCACCCAGATGAGTTCGGAACTGAG |
| | | AAGatggtgagcaagggcgaggagctgttcaccggggtggtgcccatcc |
| | | tggtcgagctggacggcgacgtaaacggccacaagttcagcgtgtccgg |
| | | cgagggcgagggcgatgccacctacggcaagctgaccctgaagttcatc |
| | | tgcaccaccggcaagctgcccgtgccctggcccaccctcgtgaccaccc |
| | | tgacctacggcgtgcagtgcttcagccgctaccccgaccacatgaagca |
| | | gcacgacttcttcaagtccgccatgcccgaaggctacgtccaggagcgc |
| | | accatcttcttcaaggacgacggcaactacaagacccgcgccgaggtga |
| | | agttcgagggcgacaccctggtgaaccgcatcgagctgaagggcatcga |
| | | cttcaaggaggacggcaacatcctggggcacaagctggagtacaactac |
| | | aacagccacaacgtctatatcatggccgacaagcagaagaacggcatca |
| | | aggtgaacttcaagatccgccacaacatcgaggacggcagcgtgcagct |
| | | cgccgaccactaccagcagaacacccccatcggcgacggccccgtgctg |
| | | ctgcccgacaaccactacctgagcacccagtccgccctgagcaaagacc |
| | | ccaacgagaagcgcgatcacatggtcctgctggagttcgtgaccgccgc |
| | | cgggatcactctcggcatggacgagctgtacaagtaagcggccgcatac |
| | | tggattggtgaatcaatgagccgtagcacaatggttacattcggctagc |
| | | taagatccaatggcaaggaccaagtgctggaacttgttttgctttagca |
| | | gatcttagcgtgagaggtatttgtcctctgtcaggagtagatagtagat |
| | | gttctttttaaactaaaatgctaactgttccgaattcctcatcgcagct |
| | | aatccgtacatcaaaagacaaaatgctaggtatgtgtactacatctcct |
| | | gttgctagataagacatatgataggaaacacaccatcaatagtcattgt |
| | | agctttacttatactacgcatttgcactttcccctgagtggcagaggcg |
| | | cattgagaaaatcgatctcaacatagtttatgtagcatcccctagatcc |
| | | attactttaagtctccttcgtctttggtgtaggcatgttggacacaacg |
| | | aggtaaaacacaacacaaacaatgtgtccagcaaagtagtagctgctcc |
| | | agttctccc |

1. A composition, comprising a sequence having at least 80% sequence identity with any of SEQ ID NOs: 3, 12, 13, 14, 15, 16, 17, 18, and 19.

2. The composition of claim 1, wherein the sequence has at least 85%, at least 90%, or at least 95% sequence identity with any of SEQ ID NOs: 3, 12, 13, 14, 15, 16, 17, 18, and 19.

3. The composition of claim 1, wherein the sequence comprises any of SEQ ID NOs: 3, 12, 13, 14, 15, 16, 17, 18, and 19.

4. The composition of any one of claim 1, wherein the sequence is part of a fusion protein.

5-9. (canceled)

10. A method of treating an animal, comprising administering a therapeutically effective amount of at least one protein derived from a microbe to the animal.

11. The method of claim 10, wherein the at least one protein derived from a microbe comprises flagellin.

12. The method of claim 10, wherein the flagellin comprises a sequence having at least 80% sequence identity with SEQ ID NO: 4.

13. The method of claim 10, wherein the flagellin comprises a sequence having at least 85%, at least 90%, at least 95% with SEQ ID NO: 4.

14. The method of claim 10, wherein the flagellin comprises SEQ ID NO: 4.

15. The method of claim 10, wherein the flagellin comprises a codon-optimized flagellin.

16. The method of claim 10, wherein the codon-optimized flagellin comprises at least 80% sequence identity with any one or more of SEQ ID NO: 3, 12, 13, 14, 15, 16, 17, 18, and 19.

17. The method of claim 10, wherein the codon-optimized flagellin comprises at least 85%, at least 90%, at least 95% sequence identity with any of one or more of SEQ ID NO: 3, 12, 13, 14, 15, 16, 17, 18, and 19.

18. (canceled)

19. The method of any of claim 10, wherein the flagellin is incorporated into at least one diatom.

20. The method of claim 33, wherein the at least one diatom comprises any one or more of Thalassiosira pseudonana, Thalassiosira weissflogii, Cyclotella cryptica, or Chaetoceros muelleri.

21-32. (canceled)

33. A method of treating an animal, comprising:
adding at least one protein from a microbe to a diatom cell; and
administering the diatom cell to the animal.

34. The method of claim 33, further comprising incorporating the diatom cell into animal feed, and administering the diatom cell and the animal feed to the animal.

35. The method of claim 33, wherein the animal comprises shrimp.

36. The method of claim 33, wherein the animal comprises fish.

37-56. (canceled)


SEQUENCE LISTING (19 sequences)

SEQ ID NO: 1 - DNA, 500 bp, synthetic construct
  source 1..500: mol_type = other DNA; note = Nitrate Reductase Promoter
agagggcaattgattgcggtatatgaaacgtgcttcttgttgctgtaaaaataactcgtcgaacaagtcatccgactagcgcctctggtgatcaaaccacgcacgtacggattggtttgatggttatcctcggattggtatccttagaacatcttcgaaggttctaggagactacccgcaaaacggatggtatttccccccaacatccaaaattggaatgccacgtcatctgcacttccggactctggcagcagcaaaatggcgcgctcgaccacatcacggttctccaccctcttccgacagcaccaacatctctttccggaagaaccaagctggaggatgaagatgacgatgagaagaggtgtggacggggtgaaaagaggctcgggaaggcttttcggcaagcacagagcttttgcacataacaacctctcacaattcaccaacccatccaattgacagcctccatcttttaacatttcaacacaaaggaaccaaca

SEQ ID NO: 2 - DNA, 300 bp, synthetic construct
  source 1..300: mol_type = other DNA; note = Nitrate Reductase Terminator
tgaggatgctcatcgttttgactttgttggatgacgcctcttgtacatactgattagacatgggagtgttggttgaattgtagatagtttacaccggctgttaactgaagagatactattacactattcatcatcattccaattgatacgtcgccgccacgcactgctcatccatagaaccctcagacacacttcacctcaaaggttcgtatctccatcggctccaaagaaaccacactgttgccttgctccagcttacgactatcctccttgaaatcatcatcgttccaccactgaaac

SEQ ID NO: 3 - DNA, 1485 bp, synthetic construct
  source 1..1485: mol_type = other DNA; note = Nucleotide sequence of Flagellin (codon-optimized)
atggcacaagtcatcaacaccaactctctctctctcttgacccagaacaacctcaacaagtcccagtccgctctcggaaccgctatcgagcgtctctcttccggactccgtatcaactctgctaaggacgatgctgcaggacaggctatcgctaaccgtttcaccgctaacatcaagggactcactcaggcttcccgtaacgctaacgacggaatctccatcgctcagaccactgagggagctctcaacgagatcaacaacaacctccagcgtgtgcgtgagctcgctgttcagtctgctaactctaccaactcccagtctgacctcgactccatccaggctgagatcacccagcgtctcaacgagatcgaccgtgtgtccggacagactcagttcaacggagtgaaggtcctcgctcaggacaacaccctcaccatccaggttggagccaacgacggagagactatcgatatcgatctcaagcagatcaactctcagaccctcggactcgataccctcaacgtgcaacaaaagtacaaggtctctgataccgctgcaactgttaccggatacgccgatactaccatcgctctcgacaacagtactttcaaggcctctgctactggactcggaggaactgaccagaagatcgatggagatctcaagttcgatgataccactggaaagtactacgccaaggttaccgttaccggaggaactggaaaggatggatactacgaggtttccgttgataagaccaacggaaaggtgactctcgctggaggagctacttccccactcaccggaggactccctgctaccgcaactgaggatgtgaagaacgtgcaagttgcaaacgctgatttgaccgaggctaaggccgcattgaccgcagcaggagttaccggaaccgcatctgttgttaagatgtcttacactgataacaacggaaagactatcgatggaggactcgcagttaaggtgggagatgattactactctgcaactcaaaacaaggatggatccatcagtatcaacactaccaagtacactgcagatgacggaacctccaagactgcactcaacaagctcggaggagcagacggaaagaccgaggttgtttctatcggaggaaagacttacgctgcaagtaaggccgagggacacaacttcaaggcacagcctgatctcgctgaggctgctgctaccaccaccgagaacccactccagaagatcgatgctgctttggcacaggttgacaccctccgttctgacctcggagctgtgcagaaccgtttcaactccgctatcaccaacctcggaaacaccgtgaacaacctcacttctgcccgttctcgtatcgaggattccgactacgctaccgaggtttccaacatgtctcgtgctcagatcctccagcaggccggaacctccgttctcgctcaggctaaccaggttccacaaaacgtcctctctctcctccgt

SEQ ID NO: 4 - protein, 495 aa, synthetic construct
  source 1..495: note = Amino acid sequence of Flagellin
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGKVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 5 - DNA, 501 bp, synthetic construct
  source 1..501: mol_type = other DNA; note = Gene cloned [Vibrio parahaemolyticus OMP (codon-optimized)]
atgtgttcttccttgccagaggagttgaacgcaagtaccgagcaagtggttactgattacaaggcattcgctgagtctcagggagagttgaccaacgacgttcgtctcggaggaatcatcgcaaaggtcgataacttcaaggacaagactcgtttggagatcgtgaacctcccaatcaacaagtctggaaagccagacatcgaccaagagcctaccggacgtttcgctgtgtacttcgatggatacttggagcctgttgccttctcccagggacgtctcgttactatcgtcggaaagggagccggagaggaggagggaaagatcggagagcacgagtacgtgttcccattggttaagggacaaggataccgtctctggaagatcgaggagcgtgtgcgtatgtacgacagtccaacctacttctacccttgttactctatcaactgccgtatgctccgtaacgatttccctcaagacggaaaggtcatcaagcaggtgaag

SEQ ID NO: 6 - protein, 167 aa, synthetic construct
  source 1..167: note = Gene cloned [Vibrio OMP]
MCSSLPEELNASTEQVVTDYKAFAESQGELTNDVRLGGIIAKVDNFKDKTRLEIVNLPINKSGKPDIDQEPTGRFAVYFDGYLEPVAFSQGRLVTIVGKGAGEEEGKIGEHEYVFPLVKGQGYRLWKIEERVRMYDSPTYFYPCYSINCRMLRNDFPQDGKVIKQVK

SEQ ID NO: 7 - DNA, 720 bp, synthetic construct
  source 1..720: clone_lib = eGFP tag; mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 8 - protein, 239 aa, synthetic construct
  source 1..239: note = eGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 9 - DNA, 375 bp, synthetic construct
  source 1..375: mol_type = other DNA; note = Sh ble (codon-optimized) selectable marker
atggctaagttgacttccgccgttcccgttttgaccgctcgtgatgtggcaggagcagttgagttttggaccgaccgtcttggattcagtcgtgatttcgttgaggatgactttgcaggagtggttagggatgacgtcacgcttttcatctcggcagtgcaagatcaggtcgtaccagacaacaccctcgcttgggtctgggttcgtggactcgatgagttgtacgccgagtggtctgaagtggtttccaccaattttcgtgacgcttctggaccagccatgactgagattggagaacaaccttggggaagggaatttgccttgagggaccccgcaggaaactgtgttcactttgtggctgaggagcaggattga

SEQ ID NO: 10 - protein, 124 aa, synthetic construct
  source 1..124: note = Sh ble
MAKLTSAVPVLTARDVAGAVEFWTDRLGFSRDFVEDDFAGVVRDDVTLFISAVQDQVVPDNTLAWVWVRGLDELYAEWSEVVSTNFRDASGPAMTEIGEQPWGREFALRDPAGNCVHFVAEEQD

SEQ ID NO: 11 - DNA, 4792 bp, synthetic construct
  source 1..4792: mol_type = other DNA; note = FCP promoter and terminator Sh ble
gggggatccactagttctagagcggccggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacgtaccgggccccccctcgaggtcgacggtatcgataagcttgcgctttttccgagaactccccataagtcaacggctccaatcaagaatgtatccgacaacggcgagcatagcaacacgtccgtctttggagtagaatcatcatgttgtggatgaatacacagatgaatgacattaaaagcatgaacatgttagagagtaggaggtagagattgatatggtagcattgcgatgtttgtttttggtcagcatatgatgagtggataccaatatgatgaaagttgaatctcgcgtttgagctcagcggtacgttattgatcgaaagtagcctgatcaaaatccttggagagtacaagaggatcaaagaatccagtgggggcgataactccaagctcgttctcaaagaggcaatggaggtagaaactcatcccagttgagaagaagtgaaggcagtggcggtggcgaaagcagaggcaacgaggacagacttcctgtgggttgatgcaacgaatatttccagaaggagaagtttagagagttgaaccgctacctacaatgacaaagtatcgtatcgattttgatgttggttggttatgaattcaaactgtaagttggattgtgagaagatcagaagttgaacgaacacatctttccgatcattcacctccacactgcaacaacacggtacttcttccgcggcaggtctctgtcgccattctcttgtcctgttgttggctgtgagacgaggaaagcaacgacaagtttcacaaaagggagttcctttaacgagatatgttttttataaagagtcccaatagaaagacaaattgattcctccgtgcaaacgcgcaaataaacaccacgtccattatatccatatctttcagagtatccaacaagtgttgaaggacaggtagttgaagtaacgtatcttccccctcgactggatccatcaacaaggcgaacaaatccattcaacctctcataaattatctgatttaccaaaccatggctaagttgacttccgccgttcccgttttgaccgctcgtgatgtggcaggagcagttgagttttggaccgaccgtcttggattcagtcgtgatttcgttgaggatgactttgcaggagtggttagggatgacgtcacgcttttcatctcggcagtgcaagatcaggtcgtaccagacaacaccctcgcttgggtctgggttcgtggactcgatgagttgtacgccgagtggtctgaagtggtttccaccaattttcgtgacgcttctggaccagccatgactgagattggagaacaaccttggggaagggaatttgccttgagggaccccgcaggaaactgtgttcactttgtggctgaggagcaggattgaatactggattggtgaatcaatgagccgtagcacaatggttacattcggctagctaagatccaatggcaaggaccaagtgctggaacttgttttgctttagcagatcttagcgtgagaggtatttgtcctctgtcaggagtagatagtagatgttctttttaaactaaaatgctaactgttccgaattcctcatcgcagctaatccgtacatcaaaagacaaaatgctaggtatgtgtactacatctcctgttgctagataagacatatgataggaaacacaccatcaatagtcattgtagctttacttatactacgcatttgcactttcccctgagtggcagaggcgcattgagaaaatcgatctcaacatagtttatgtagcatcccctagatccattactttaagtctccttcgtctttggtgtaggcatgttggacacaacgaggtaaaacacaacacaaacaatgtgtccagcaaagtagtagctgctccagttctccc

SEQ ID NO: 12 - DNA, 6670 bp, synthetic construct
  source 1..6670: mol_type = other DNA; note = Flagellin + Gene 1. Vibrio parahaemolyticus Slp/YeaY family lipoprotein
cgggggatccactagttctagagcggccggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggcccccccagagggcaattgattgcggtatatgaaacgtgcttcttgttgctgtaaaaataactcgtcgaacaagtcatccgactagcgcctctggtgatcaaaccacgcacgtacggattggtttgatggttatcctcggattggtatccttagaacatcttcgaaggttctaggagactacccgcaaaacggatggtatttccccccaacatccaaaattggaatgccacgtcatctgcacttccggactctggcagcagcaaaatggcgcgctcgaccacatcacggttctccaccctcttccgacagcaccaacatctctttccggaagaaccaagctggaggatgaagatgacgatgagaagaggtgtggacggggtgaaaagaggctcgggaaggcttttcggcaagcacagagcttttgcacataacaacctctcacaattcaccaacccatccaattgacagcctccatcttttaacatttcaacacaaaggaaccaacagatatcataatcatggcacaagtcatcaacaccaactctctctctctcttgacccagaacaacctcaacaagtcccagtccgctctcggaaccgctatcgagcgtctctcttccggactccgtatcaactctgctaaggacgatgctgcaggacaggctatcgctaaccgtttcaccgctaacatcaagggactcactcaggcttcccgtaacgctaacgacggaatctccatcgctcagaccactgagggagctctcaacgagatcaacaacaacctccagcgtgtgcgtgagctcgctgttcagtctgctaactctaccaactcccagtctgacctcgactccatccaggctgagatcacccagcgtctcaacgagatcgaccgtgtgtccggacagactcagttcaacggagtgaaggtcctcgctcaggacaacaccctcaccatccaggttggagccaacgacggagagactatcgatatcgatctcaagcagatcaactctcagaccctcggactcgataccctcaacgtgcaacaaaagtacaaggtctctgataccgctgcaactgttaccggatacgccgatactaccatcgctctcgacaacagtactttcaaggcctctgctactggactcggaggaactgaccagaagatcgatggagatctcaagttcgatgataccactggaaagtactacgccaaggttaccgttaccggaggaactggaaaggatggatactacgaggtttccgttgataagaccaacggaaaggtgactctcgctggaggagctacttccccactcaccggaggactccctgctaccgcaactgaggatgtgaagaacgtgcaagttgcaaacgctgatttgaccgaggctaaggccgcattgaccgcagcaggagttaccggaaccgcatctgttgttaagatgtcttacactgataacaacggaaagactatcgatggaggactcgcagttaaggtgggagatgattactactctgcaactcaaaacaaggatggatccatcagtatcaacactaccaagtacactgcagatgacggaacctccaagactgcactcaacaagctcggaggagcagacggaaagaccgaggttgtttctatcggaggaaagacttacgctgcaagtaaggccgagggacacaacttcaaggcacagcctgatctcgctgaggctgctgctaccaccaccgagaacccactccagaagatcgatgctgctttggcacaggttgacaccctccgttctgacctcggagctgtgcagaaccgtttcaactccgctatcaccaacctcggaaacaccgtgaacaacctcacttctgcccgttctcgtatcgaggattccgactacgctaccgaggtttccaacatgtctcgtgctcagatcctccagcaggccggaacctccgttctcgctcaggctaaccaggttccacaaaacgtcctctctctcctccgtatgtgttcttccttgccagaggagttgaacgcaagtaccgagcaagtggttactgattacaaggcattcgctgagtctcagggagagttgaccaacgacgttcgtctcggaggaatcatcgcaaaggtcgataacttcaaggacaagactcgtttggagatcgtgaacctcccaatcaacaagtctggaaagccagacatcgaccaagagcctaccggacgtttcgctgtgtacttcgatggatacttggagcctgttgccttctcccagggacgtctcgttactatcgtcggaaagggagccggagaggaggagggaaagatcggagagcacgagtacgtgttcccattggttaagggacaaggataccgtctctggaagatcgaggagcgtgtgcgtatgtacgacagtccaacctacttctacccttgttactctatcaactgccgtatgctccgtaacgatttccctcaagacggaaaggtcatcaagcaggtgaagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgctgaggatgctcatcgttttgactttgttggatgacgcctcttgtacatactgattagacatgggagtgttggttgaattgtagatagtttacaccggctgttaactgaagagatactattacactattcatcatcattccaattgatacgtcgccgccacgcactgctcatccatagaaccctcagacacacttcacctcaaaggttcgtatctccatcggctccaaagaaaccacactgttgccttgctccagcttacgactatcctccttgaaatcatcatcgttccaccactgaaactgtctctgttcccactcatctcttctctgattaccagagagtgtcgtttccgtgatggagactactttgcgagaaggaaacaagacgccaagatccacctcaaccggaagggagtattgatgactttctcccactccgtactgatgcccaagcctgacaagataggtgtcgtcttgctctagtgatgcaaacgttaccagcatgatattatcaggcaatgcatcc

SEQ ID NO: 13 - DNA, 7294 bp, synthetic construct
  source 1..7294: mol_type = other DNA; note = Flagellin + Gene 2. Somatotropin)
cgggggatccactagttctagagcggccggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggcagttgcctaccgaaaagctcacggagatgaagaatggacgccctttcattttgaagcggaggatatggaagaagtgacggggagtaacggagctcattatgctgctcatacgggaaatgttgagactttgatgcatatcatccagaatgagagaattgatatggtgcatgaaacggatgggaatggatggctgcctattcatgaggctgcacgaagtggtcatgtggagatcgtgcagattctgattgagcatggggcggatgtggatagtaggacggaatggggggagggacattcggtgttgaacattgcgacgcagtaccacgaggaggatagttcaatcattcagtacttgttgagtttgggggcgatgaatatcattccttggcctgaggaggaggagctttagatgcgacaacatgagatgttttaggagtgtgtggggtaggaaggagaagtatctagtattgcaagggtcgatgtttggaagtcagacggatcaattacgatctatactggagttgcagtggacaagaacatgtgggacctcgataaaggatatggtgcttgggcgggtgtcatcagatacactgcctagagggcaattgattgcggtatatgaaacgtgcttcttgttgctgtaaaaataactcgtcgaacaagtcatccgactagcgcctctggtgatcaaaccacgcacgtacggattggtttgatggttatcctcggattggtatccttagaacatcttcgaaggttctaggagactacccgcaaaacggatggtatttccccccaacatccaaaattggaatgccacgtcatctgcacttccggactctggcagcagcaaaatggcgcgctcgaccacatcacggttctccaccctcttccgacagcaccaacatctctttccggaagaaccaagctggaggatgaagatgacgatgagaagaggtgtggacggggtgaaaagaggctcgggaaggcttttcggcaagcacagagcttttgcacataacaacctctcacaattcaccaacccatccaattgacagcctccatcttttaacatttcaacacaaaggaaccaacagatatcataatcatggcacaagtcatcaacaccaactctctctctctcttgacccagaacaacctcaacaagtcccagtccgctctcggaaccgctatcgagcgtctctcttccggactccgtatcaactctgctaaggacgatgctgcaggacaggctatcgctaaccgtttcaccgctaacatcaagggactcactcaggcttcccgtaacgctaacgacggaatctccatcgctcagaccactgagggagctctcaacgagatcaacaacaacctccagcgtgtgcgtgagctcgctgttcagtctgctaactctaccaactcccagtctgacctcgactccatccaggctgagatcacccagcgtctcaacgagatcgaccgtgtgtccggacagactcagttcaacggagtgaaggtcctcgctcaggacaacaccctcaccatccaggttggagccaacgacggagagactatcgatatcgatctcaagcagatcaactctcagaccctcggactcgataccctcaacgtgcaacaaaagtacaaggtctctgataccgctgcaactgttaccggatacgccgatactaccatcgctctcgacaacagtactttcaaggcctctgctactggactcggaggaactgaccagaagatcgatggagatctcaagttcgatgataccactggaaagtactacgccaaggttaccgttaccggaggaactggaaaggatggatactacgaggtttccgttgataagaccaacggaaaggtgactctcgctggaggagctacttccccactcaccggaggactccctgctaccgcaactgaggatgtgaagaacgtgcaagttgcaaacgctgatttgaccgaggctaaggccgcattgaccgcagcaggagttaccggaaccgcatctgttgttaagatgtcttacactgataacaacggaaagactatcgatggaggactcgcagttaaggtgggagatgattactactctgcaactcaaaacaaggatggatccatcagtatcaacactaccaagtacactgcagatgacggaacctccaagactgcactcaacaagctcggaggagcagacggaaagaccgaggttgtttctatcggaggaaagacttacgctgcaagtaaggccgagggacacaacttcaaggcacagcctgatctcgctgaggctgctgctaccaccaccgagaacccactccagaagatcgatgctgctttggcacaggttgacaccctccgttctgacctcggagctgtgcagaaccgtttcaactccgctatcaccaacctcggaaacaccgtgaacaacctcacttctgcccgttctcgtatcgaggattccgactacgctaccgaggtttccaacatgtctcgtgctcagatcctccagcaggccggaacctccgttctcgctcaggctaaccaggttccacaaaacgtcctctctctcctccgtatgcaaccaatcaccgagaaccagcgtttgttctccatcgctgtcggacgtgtgcaatacttgcacctcgtcgccaagaagctcttctccgatttcgagaacagtttgcaattggaggaccagcgtttgctcaacaagatcgcatctaaggagttctgccactctgataacttcttgtcccctatcgacaagcatgagactcaaggaagttctgtccagaagttgctctctgtgtcctaccgtttgatcgagagttgggagttcttctctcgtttcctcgtggcttctttcgccgttcgtacccaggtcacttctaagctctctgagttgaagatgggattgctcaagttgatcgaggcaaaccaagatggagctggaggattctccgagtccagtgttttgcagctcaccccatacggaaacagtgagttgttcgcctgtttcaagaaggacatgcataaggttgagacttacttgactgtcgccaagtgtcgtctcttccctgaggcaaactgcactctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgctgaggatgctcatcgttttgactttgttggatgacgcctcttgtacatactgattagacatgggagtgttggttgaattgtagatagtttacaccggctgttaactgaagagatactattacactattcatcatcattccaattgatacgtcgccgccacgcactgctcatccatagaaccctcagacacacttcacctcaaaggttcgtatctccatcggctccaaagaaaccacactgttgccttgctccagcttacgactatcctccttgaaatcatcatcgttccaccactgaaactgtctctgttcccactcatctcttctctgattaccagagagtgtcgtttccgtgatggagactactttgcgagaaggaaacaagacgccaagatccacctcaaccggaagggagtattgatgactttctcccactccgtactgatgcccaagcctgacaagataggtgtcgtcttgctctagtgatgcaaacgttaccagcatgatattatcaggcaatgcatcc

SEQ ID NO: 14 - DNA, 6502 bp, synthetic construct
  source 1..6502: mol_type = other DNA; note = Flagellin + Gene 3. Vibrio parahaemolyticus pirAVP)
cgggggatccactagttctagagcggccggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggcccccccagagggcaattgattgcggtatatgaaacgtgcttcttgttgctgtaaaaataactcgtcgaacaagtcatccgactagcgcctctggtgatcaaaccacgcacgtacggattggtttgatggttatcctcggattggtatccttagaacatcttcgaaggttctaggagactacccgcaaaacggatggtatttccccccaacatccaaaattggaatgccacgtcatctgcacttccggactctggcagcagcaaaatggcgcgctcgaccacatcacggttctccaccctcttccgacagcaccaacatctctttccggaagaaccaagctggaggatgaagatgacgatgagaagaggtgtggacggggtgaaaagaggctcgggaaggcttttcggcaagcacagagcttttgcacataacaacctctcacaattcaccaacccatccaattgacagcctccatcttttaacatttcaacacaaaggaaccaacagatatcataatcatggcacaagtcatcaacaccaactctctctctctcttgacccagaacaacctcaacaagtcccagtccgctctcggaaccgctatcgagcgtctctcttccggactccgtatcaactctgctaaggacgatgctgcaggacaggctatcgctaaccgtttcaccgctaacatcaagggactcactcaggcttcccgtaacgctaacgacggaatctccatcgctcagaccactgagggagctctcaacgagatcaacaacaacctccagcgtgtgcgtgagctcgctgttcagtctgctaactctaccaactcccagtctgacctcgactccatccaggctgagatcacccagcgtctcaacgagatcgaccgtgtgtccggacagactcagttcaacggagtgaaggtcctcgctcaggacaacaccctcaccatccaggttggagccaacgacggagagactatcgatatcgatctcaagcagatcaactctcagaccctcggactcgataccctcaacgtgcaacaaaagtacaaggtctctgataccgctgcaactgttaccggatacgccgatactaccatcgctctcgacaacagtactttcaaggcctctgctactggactcggaggaactgaccagaagatcgatggagatctcaagttcgatgataccactggaaagtactacgccaaggttaccgttaccggaggaactggaaaggatggatactacgaggtttccgttgataagaccaacggaaaggtgactctcgctggaggagctacttccccactcaccggaggactccctgctaccgcaactgaggatgtgaagaacgtgcaagttgcaaacgctgatttgaccgaggctaaggccgcattgaccgcagcaggagttaccggaaccgcatctgttgttaagatgtcttacactgataacaacggaaagactatcgatggaggactcgcagttaaggtgggagatgattactactctgcaactcaaaacaaggatggatccatcagtatcaacactaccaagtacactgcagatgacggaacctccaagactgcactcaacaagctcggaggagcagacggaaagaccgaggttgtttctatcggaggaaagacttacgctgcaagtaaggccgagggacacaacttcaaggcacagcctgatctcgctgaggctgctgctaccaccaccgagaacccactccagaagatcgatgctgctttggcacaggttgacaccctccgttctgacctcggagctgtgcagaaccgtttcaactccgctatcaccaacctcggaaacaccgtgaacaacctcacttctgcccgttctcgtatcgaggattccgactacgctaccgaggtttccaacatgtctcgtgctcagatcctccagcaggccggaacctccgttctcgctcaggctaaccaggttccacaaaacgtcctctctctcctccgtatgtctaacaacatcaagcatgagactgattactctcacgattggaccgtcgagccaaacggaggagtgaccgaggtggattctaagcataccccaatcatccctgaggtgggacgttctgtcgatatcgagaacactggacgtggagagttgaccatccaataccaatggggagccccattcatggctggaggatggaaggttgccaagtctcatgtcgtgcaacgtgatgagacctaccatttgcaacgtcctgataacgctttctaccaccaacgtatcgttgtgatcaacaacggagcctctcgtggattctgtaccatctactaccatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgctgaggatgctcatcgttttgactttgttggatgacgcctcttgtacatactgattagacatgggagtgttggttgaattgtagatagtttacaccggctgttaactgaagagatactattacactattcatcatcattccaattgatacgtcgccgccacgcactgctcatccatagaaccctcagacacacttcacctcaaaggttcgtatctccatcggctccaaagaaaccacactgttgccttgctccagcttacgactatcctccttgaaatcatcatcgttccaccactgaaactgtctctgttcccactcatctcttctctgattaccagagagtgtcgtttccgtgatggagactactttgcgagaaggaaacaagacgccaagatccacctcaaccggaagggagtattgatgactttctcccactccgtactgatgcccaagcctgacaagataggtgtcgtcttgctctagtgatgcaaacgttaccagcatgatattatcaggcaatgcatcc

SEQ ID NO: 15 - DNA, 7483 bp, synthetic construct
  source 1..7483: mol_type = other DNA; note = Flagellin + Gene 4. Vibrio parahaemolyticus pirBVP)
cgggggatccactagttctagagcggccggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggcccccccagagggcaattgattgcggtatatgaaacgtgcttcttgttgctgtaaaaataactcgtcgaacaagtcatccgactagcgcctctggtgatcaaaccacgcacgtacggattggtttgatggttatcctcggattggtatccttagaacatcttcgaaggttctaggagactacccgcaaaacggatggtatttccccccaacatccaaaattggaatgccacgtcatctgcacttccggactctggcagcagcaaaatggcgcgctcgaccacatcacggttctccaccctcttccgacagcaccaacatctctttccggaagaaccaagctggaggatgaagatgacgatgagaagaggtgtggacggggtgaaaagaggctcgggaaggcttttcggcaagcacagagcttttgcacataacaacctctcacaattcaccaacccatccaattgacagcctccatcttttaacatttcaacacaaaggaaccaacagatatcataatcatggcacaagtcatcaacaccaactctctctctctcttgacccagaacaacctcaacaagtcccagtccgctctcggaaccgctatcgagcgtctctcttccggactccgtatcaactctgctaaggacgatgctgcaggacaggctatcgctaaccgtttcaccgctaacatcaagggactcactcaggcttcccgtaacgctaacgacggaatctccatcgctcagaccactgagggagctctcaacgagatcaacaacaacctccagcgtgtgcgtgagctcgctgttcagtctgctaactctaccaactcccagtctgacctcgactccatccaggctgagatcacccagcgtctcaacgagatcgaccgtgtgtccggacagactcagttcaacggagtgaaggtcctcgctcaggacaacaccctcaccatccaggttggagccaacgacggagagactatcgatatcgatctcaagcagatcaactctcagaccctcggactcgataccctcaacgtgcaacaaaagtacaaggtctctgataccgctgcaactgttaccggatacgccgatactaccatcgctctcgacaacagtactttcaaggcctctgctactggactcggaggaactgaccagaagatcgatggagatctcaagttcgatgataccactggaaagtactacgccaaggttaccgttaccggaggaactggaaaggatggatactacgaggtttccgttgataagaccaacggaaaggtgactctcgctggaggagctacttccccactcaccggaggactccctgctaccgcaactgaggatgtgaagaacgtgcaagttgcaaacgctgatttgaccgaggctaaggccgcattgaccgcagcaggagttaccggaaccgcatctgttgttaagatgtcttacactgataacaacggaaagactatcgatggaggactcgcagttaaggtgggagatgattactactctgcaactcaaaacaaggatggatccatcagtatcaacactaccaagtacactgcagatgacggaacctccaagactgcactcaacaagctcggaggagcagacggaaagaccgaggttgtttctatcggaggaaagacttacgctgcaagtaaggccgagggacacaacttcaaggcacagcctgatctcgctgaggctgctgctaccaccaccgagaacccactccagaagatcgatgctgctttggcacaggttgacaccctccgttctgacctcggagctgtgcagaaccgtttcaactccgctatcaccaacctcggaaacaccgtgaacaacctcacttctgcccgttctcgtatcgaggattccgactacgctaccgaggtttccaacatgtctcgtgctcagatcctccagcaggccggaacctccgttctcgctcaggctaaccaggttccacaaaacgtcctctctctcctccgtatgactaacgagtacgttgtgactatgtcttctctcactgagttcaacccaaacaacgctcgtaagtcttacctcttcgataactacgaggttgatcctaactacgccttcaaggccatggtttctttcggattgtctaacatcccatacgccggaggattcctctctactttgtggaacatcttctggccaaacactccaaacgagcctgatatcgagaacatctgggagcaattgcgtgatcgtatccaagatctcgttgatgagtctatcatcgatgccatcaacggaatcctcgattctaagatcaaggagactcgtgataagatccaagatatcaacgagaccatcgagaacttcggatacgctgccgctaaggatgattacatcggactcgtgactcactacttgatcggattggaggagaacttcaagcgtgagctcgatggagatgagtggttgggatacgctatcttgcctctcttggccaccactgtctctttgcaaatcacctacatggcttgtggattggattacaaggatgagttcggattcactgattctgatgtccataagctcacccgtaacatcgataagttgtacgatgatgtttcttcttacatcactgagttggccgcttgggccgataacgattcttacaacaacgccaaccaagataacgtttacgatgaggttatgggagcccgttcttggtgtaccgtgcatggattcgagcatatgttgatctggcaaaagatcaaggagttgaagaaggtggatgtgttcgtgcactctaacctcatctcttactctccagctgtgggattcccatctggaaacttcaactacatcgctactggaaccgaggatgagatccctcaaccactcaagccaaacatgttcggagagcgtcgtaaccgtatcgtgaagatcgagtcttggaactctatcgagatccactactacaaccgtgttggacgtttgaagctcacctacgagaacggagaggtggtcgagttgggaaaggcccataagtacgatgagcattaccaatctatcgagctcaacggagcctacatcaagtacgttgatgtgatcgccaacggacctgaggctatcgatcgtatcgtcttccacttctctgatgatcgtaccttcgtggttggagagaactctggaaagccatctgtgcgtctccaactcgagggacacttcatctgtggaatgttggctgatcaagagggatctgataaggttgccgctttctctgtcgcttacgagctcttccacccagatgagttcggaactgagaagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgctgaggatgctcatcgttttgactttgttggatgacgcctcttgtacatactgattagacatgggagtgttggttgaattgtagatagtttacaccggctgttaactgaagagatactattacactattcatcatcattccaattgatacgtcgccgccacgcactgctcatccatagaaccctcagacacacttcacctcaaaggttcgtatctccatcggctccaaagaaaccacactgttgccttgctccagcttacgactatcctccttgaaatcatcatcgttccaccactgaaactgtctctgttcccactcatctcttctctgattaccagagagtgtcgtttccgtgatggagactactttgcgagaaggaaacaagacgccaagatccacctcaaccggaagggagtattgatgactttctcccactccgtactgatgcccaagcctgacaagataggtgtcgtcttgctctagtgatgcaaacgttaccagcatgatattatcaggcaatgcatcc

SEQ ID NO: 16 - DNA, 7134 bp, synthetic construct
  source 1..7134: mol_type = other DNA; note = Flagellin + Gene 1. (Vibrio parahaemolyticus Slp-YeaY family lipoprotein)
gggggatccactagttctagagcggccggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacgtaccgggccccccctcgaggtcgacggtatcgataagcttgcgctttttccgagaactccccataagtcaacggctccaatcaagaatgtatccgacaacggcgagcatagcaacacgtccgtctttggagtagaatcatcatgttgtggatgaatacacagatgaatgacattaaaagcatgaacatgttagagagtaggaggtagagattgatatggtagcattgcgatgtttgtttttggtcagcatatgatgagtggataccaatatgatgaaagttgaatctcgcgtttgagctcagcggtacgttattgatcgaaagtagcctgatcaaaatccttggagagtacaagaggatcaaagaatccagtgggggcgataactccaagctcgttctcaaagaggcaatggaggtagaaactcatcccagttgagaagaagtgaaggcagtggcggtggcgaaagcagaggcaacgaggacagacttcctgtgggttgatgcaacgaatatttccagaaggagaagtttagagagttgaaccgctacctacaatgacaaagtatcgtatcgattttgatgttggttggttatgaattcaaactgtaagttggattgtgagaagatcagaagttgaacgaacacatctttccgatcattcacctccacactgcaacaacacggtacttcttccgcggcaggtctctgtcgccattctcttgtcctgttgttggctgtgagacgaggaaagcaacgacaagtttcacaaaagggagttcctttaacgagatatgttttttataaagagtcccaatagaaagacaaattgattcctccgtgcaaacgcgcaaataaacaccacgtccattatatccatatctttcagagtatccaacaagtgttgaaggacaggtagttgaagtaacgtatcttccccctcgactggatccatcaacaaggcgaacaaatccattcaacctctcataaattatctgatttaccaaaccgatatggcacaagtcatcaacaccaactctctctctctcttgacccagaacaacctcaacaagtcccagtccgctctcggaaccgctatcgagcgtctctcttccggactccgtatcaactctgctaaggacgatgctgcaggacaggctatcgctaaccgtttcaccgctaacatcaagggactcactcaggcttcccgtaacgctaacgacggaatctccatcgctcagaccactgagggagctctcaacgagatcaacaacaacctccagcgtgtgcgtgagctcgctgttcagtctgctaactctaccaactcccagtctgacctcgactccatccaggctgagatcacccagcgtctcaacgagatcgaccgtgtgtccggacagactcagttcaacggagtgaaggtcctcgctcaggacaacaccctcaccatccaggttggagccaacgacggagagactatcgatatcgatctcaagcagatcaactctcagaccctcggactcgataccctcaacgtgcaacaaaagtacaaggtctctgataccgctgcaactgttaccggatacgccgatactaccatcgctctcgacaacagtactttcaaggcctctgctactggactcggaggaactgaccagaagatcgatggagatctcaagttcgatgataccactggaaagtactacgccaaggttaccgttaccggaggaactggaaaggatggatactacgaggtttccgttgataagaccaacggaaaggtgactctcgctggaggagctacttccccactcaccggaggactccctgctaccgcaactgaggatgtgaagaacgtgcaagttgcaaacgctgatttgaccgaggctaaggccgcattgaccgcagcaggagttaccggaaccgcatctgttgttaagatgtcttacactgataacaacggaaagactatcgatggaggactcgcagttaaggtgggagatgattactactctgcaactcaaaacaaggatggatccatcagtatcaacactaccaagtacactgcagatgacggaacctccaagactgcactcaacaagctcggaggagcagacggaaagaccgaggttgtttctatcggaggaaagacttacgctgcaagtaaggccgagggacacaacttcaaggcacagcctgatctcgctgaggctgctgctaccaccaccgagaacccactccagaagatcgatgctgctttggcacaggttgacaccctccgttctgacctcggagctgtgcagaaccgtttcaactccgctatcaccaacctcggaaacaccgtgaacaacctcacttctgcccgttctcgtatcgaggattccgactacgctaccgaggtttccaacatgtctcgtgctcagatcctccagcaggccggaacctccgttctcgctcaggctaaccaggttccacaaaacgtcctctctctcctccgtatgtgttcttccttgccagaggagttgaacgcaagtaccgagcaagtggttactgattacaaggcattcgctgagtctcagggagagttgaccaacgacgttcgtctcggaggaatcatcgcaaaggtcgataacttcaaggacaagactcgtttggagatcgtgaacctcccaatcaacaagtctggaaagccagacatcgaccaagagcctaccggacgtttcgctgtgtacttcgatggatacttggagcctgttgccttctcccagggacgtctcgttactatcgtcggaaagggagccggagaggaggagggaaagatcggagagcacgagtacgtgttcccattggttaagggacaaggataccgtctctggaagatcgaggagcgtgtgcgtatgtacgacagtccaacctacttctacccttgttactctatcaactgccgtatgctccgtaacgatttccctcaagacggaaaggtcatcaagcaggtgaagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcatactggattggtgaatcaatgagccgtagcacaatggttacattcggctagctaagatccaatggcaaggaccaagtgctggaacttgttttgctttagcagatcttagcgtgagaggtatttgtcctctgtcaggagtagatagtagatgttctttttaaactaaaatgctaactgttccgaattcctcatcgcagctaatccgtacatcaaaagacaaaatgctaggtatgtgtactacatctcctgttgctagataagacatatgataggaaacacaccatcaatagtcattgtagctttacttatactacgcatttgcactttcccctgagtggcagaggcgcattgagaaaatcgatctcaacatagtttatgtagcatcccctagatccattactttaagtctccttcgtctttggtgtaggcatgttggacacaacgaggtaaaacacaacacaaacaatgtgtccagcaaagtagtagctgctccagttctccc

SEQ ID NO: 17 - DNA, 7155 bp, synthetic construct
  source 1..7155: mol_type = other DNA; note = Flagellin + Gene 2. Somatotropin)
gggggatccactagttctagagcggccggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacgtaccgggccccccctcgaggtcgacggtatcgataagcttgcgctttttccgagaactccccataagtcaacggctccaatcaagaatgtatccgacaacggcgagcatagcaacacgtccgtctttggagtagaatcatcatgttgtggatgaatacacagatgaatgacattaaaagcatgaacatgttagagagtaggaggtagagattgatatggtagcattgcgatgtttgtttttggtcagcatatgatgagtggataccaatatgatgaaagttgaatctcgcgtttgagctcagcggtacgttattgatcgaaagtagcctgatcaaaatccttggagagtacaagaggatcaaagaatccagtgggggcgataactccaagctcgttctcaaagaggcaatggaggtagaaactcatcccagttgagaagaagtgaaggcagtggcggtggcgaaagcagaggcaacgaggacagacttcctgtgggttgatgcaacgaatatttccagaaggagaagtttagagagttgaaccgctacctacaatgacaaagtatcgtatcgattttgatgttggttggttatgaattcaaactgtaagttggattgtgagaagatcagaagttgaacgaacacatctttccgatcattcacctccacactgcaacaacacggtacttcttccgcggcaggtctctgtcgccattctcttgtcctgttgttggctgtgagacgaggaaagcaacgacaagtttcacaaaagggagttcctttaacgagatatgttttttataaagagtcccaatagaaagacaaattgattcctccgtgcaaacgcgcaaataaacaccacgtccattatatccatatctttcagagtatccaacaagtgttgaaggacaggtagttgaagtaacgtatcttccccctcgactggatccatcaacaaggcgaacaaatccattcaacctctcataaattatctgatttaccaaaccgatatggcacaagtcatcaacaccaactctctctctctcttgacccagaacaacctcaacaagtcccagtccgctctcggaaccgctatcgagcgtctctcttccggactccgtatcaactctgctaaggacgatgctgcaggacaggctatcgctaaccgtttcaccgctaacatcaagggactcactcaggcttcccgtaacgctaacgacggaatctccatcgctcagaccactgagggagctctcaacgagatcaacaacaacctccagcgtgtgcgtgagctcgctgttcagtctgctaactctaccaactcccagtctgacctcgactccatccaggctgagatcacccagcgtctcaacgagatcgaccgtgtgtccggacagactcagttcaacggagtgaaggtcctcgctcaggacaacaccctcaccatccaggttggagccaacgacggagagactatcgatatcgatctcaagcagatcaactctcagaccctcggactcgataccctcaacgtgcaacaaaagtacaaggtctctgataccgctgcaactgttaccggatacgccgatactaccatcgctctcgacaacagtactttcaaggcctctgctactggactcggaggaactgaccagaagatcgatggagatctcaagttcgatgataccactggaaagtactacgccaaggttaccgttaccggaggaactggaaaggatggatactacgaggtttccgttgataagaccaacggaaaggtgactctcgctggaggagctacttccccactcaccggaggactccctgctaccgcaactgaggatgtgaagaacgtgcaagttgcaaacgctgatttgaccgaggctaaggccgcattgaccgcagcaggagttaccggaaccgcatctgttgttaagatgtcttacactgataacaacggaaagactatcgatggaggactcgcagttaaggtgggagatgattactactctgcaactcaaaacaaggatggatccatcagtatcaacactaccaagtacactgcagatgacggaacctccaagactgcactcaacaagctcggaggagcagacggaaagaccgaggttgtttctatcggaggaaagacttacgctgcaagtaaggccgagggacacaacttcaaggcacagcctgatctcgctgaggctgctgctaccaccaccgagaacccactccagaagatcgatgctgctttggcacaggttgacaccctccgttctgacctcggagctgtgcagaaccgtttcaactccgctatcaccaacctcggaaacaccgtgaacaacctcacttctgcccgttctcgtatcgaggattccgactacgctaccgaggtttccaacatgtctcgtgctcagatcctccagcaggccggaacctccgttctcgctcaggctaaccaggttccacaaaacgtcctctctctcctccgtatgcaaccaatcaccgagaaccagcgtttgttctccatcgctgtcggacgtgtgcaatacttgcacctcgtcgccaagaagctcttctccgatttcgagaacagtttgcaattggaggaccagcgtttgctcaacaagatcgcatctaaggagttctgccactctgataacttcttgtcccctatcgacaagcatgagactcaaggaagttctgtccagaagttgctctctgtgtcctaccgtttgatcgagagttgggagttcttctctcgtttcctcgtggcttctttcgccgttcgtacccaggtcacttctaagctctctgagttgaagatgggattgctcaagttgatcgaggcaaaccaagatggagctggaggattctccgagtccagtgttttgcagctcaccccatacggaaacagtgagttgttcgcctgtttcaagaaggacatgcataaggttgagacttacttgactgtcgccaagtgtcgtctcttccctgaggcaaactgcactctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcatactggattggtgaatcaatgagccgtagcacaatggttacattcggctagctaagatccaatggcaaggaccaagtgctggaacttgttttgctttagcagatcttagcgtgagaggtatttgtcctctgtcaggagtagatagtagatgttctttttaaactaaaatgctaactgttccgaattcctcatcgcagctaatccgtacatcaaaagacaaaatgctaggtatgtgtactacatctcctgttgctagataagacatatgataggaaacacaccatcaatagtcattgtagctttacttatactacgcatttgcactttcccctgagtggcagaggcgcattgagaaaatcgatctcaacatagtttatgtagcatcccctagatccattactttaagtctccttcgtctttggtgtaggcatgttggacacaacgaggtaaaacacaacacaaacaatgtgtccagcaaagtagtagctgctccagttctccc

SEQ ID NO: 18 - DNA, 6966 bp, synthetic construct
  source 1..6966: mol_type = other DNA; note = Flagellin + Gene 3. Vibrio parahaemolyticus pirAVP)
gggggatccactagttctagagcggccggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacgtaccgggccccccctcgaggtcgacggtatcgataagcttgcgctttttccgagaactccccataagtcaacggctccaatcaagaatgtatccgacaacggcgagcatagcaacacgtccgtctttggagtagaatcatcatgttgtggatgaatacacagatgaatgacattaaaagcatgaacatgttagagagtaggaggtagagattgatatggtagcattgcgatgtttgtttttggtcagcatatgatgagtggataccaatatgatgaaagttgaatctcgcgtttgagctcagcggtacgttattgatcgaaagtagcctgatcaaaatccttggagagtacaagaggatcaaagaatccagtgggggcgataactccaagctcgttctcaaagaggcaatggaggtagaaactcatcccagttgagaagaagtgaaggcagtggcggtggcgaaagcagaggcaacgaggacagacttcctgtgggttgatgcaacgaatatttccagaaggagaagtttagagagttgaaccgctacctacaatgacaaagtatcgtatcgattttgatgttggttggttatgaattcaaactgtaagttggattgtgagaagatcagaagttgaacgaacacatctttccgatcattcacctccacactgcaacaacacggtacttcttccgcggcaggtctctgtcgccattctcttgtcctgttgttggctgtgagacgaggaaagcaacgacaagtttcacaaaagggagttcctttaacgagatatgttttttataaagagtcccaatagaaagacaaattgattcctccgtgcaaacgcgcaaataaacaccacgtccattatatccatatctttcagagtatccaacaagtgttgaaggacaggtagttgaagtaacgtatcttccccctcgactggatccatcaacaaggcgaacaaatccattcaacctctcataaattatctgatttaccaaaccgatatggcacaagtcatcaacaccaactctctctctctcttgacccagaacaacctcaacaagtcccagtccgctctcggaaccgctatcgagcgtctctcttccggactccgtatcaactctgctaaggacgatgctgcaggacaggctatcgctaaccgtttcaccgctaacatcaagggactcactcaggcttcccgtaacgctaacgacggaatctccatcgctcagaccactgagggagctctcaacgagatcaacaacaacctccagcgtgtgcgtgagctcgctgttcagtctgctaactctaccaactcccagtctgacctcgactccatccaggctgagatcacccagcgtctcaacgagatcgaccgtgtgtccggacagactcagttcaacggagtgaaggtcctcgctcaggacaacaccctcaccatccaggttggagccaacgacggagagactatcgatatcgatctcaagcagatcaactctcagaccctcggactcgataccctcaacgtgcaacaaaagtacaaggtctctgataccgctgcaactgttaccggatacgccgatactaccatcgctctcgacaacagtactttcaaggcctctgctactggactcggaggaactgaccagaagatcgatggagatctcaagttcgatgataccactggaaagtactacgccaaggttaccgttaccggaggaactggaaaggatggatactacgaggtttccgttgataagaccaacggaaaggtgactctcgctggaggagctacttccccactcaccggaggactccctgctaccgcaactgaggatgtgaagaacgtgcaagttgcaaacgctgatttgaccgaggctaaggccgcattgaccgcagcaggagttaccggaaccgcatctgttgttaagatgtcttacactgataacaacggaaagactatcgatggaggactcgcagttaaggtgggagatgattactactctgcaactcaaaacaaggatggatccatcagtatcaacactaccaagtacactgcagatgacggaacctccaagactgcactcaacaagctcggaggagcagacggaaagaccgaggttgtttctatcggaggaaagacttacgctgcaagtaaggccgagggacacaacttcaaggcacagcctgatctcgctgaggctgctgctaccaccaccgagaacccactccagaagatcgatgctgctttggcacaggttgacaccctccgttctgacctcggagctgtgcagaaccgtttcaactccgctatcaccaacctcggaaacaccgtgaacaacctcacttctgcccgttctcgtatcgaggattccgactacgctaccgaggtttccaacatgtctcgtgctcagatcctccagcaggccggaacctccgttctcgctcaggctaaccaggttccacaaaacgtcctctctctcctccgtatgtctaacaacatcaagcatgagactgattactctcacgattggaccgtcgagccaaacggaggagtgaccgaggtggattctaagcataccccaatcatccctgaggtgggacgttctgtcgatatcgagaacactggacgtggagagttgaccatccaataccaatggggagccccattcatggctggaggatggaaggttgccaagtctcatgtcgtgcaacgtgatgagacctaccatttgcaacgtcctgataacgctttctaccaccaacgtatcgttgtgatcaacaacggagcctctcgtggattctgtaccatctactaccatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcatactggattggtgaatcaatgagccgtagcacaatggttacattcggctagctaagatccaatggcaaggaccaagtgctggaacttgttttgctttagcagatcttagcgtgagaggtatttgtcctctgtcaggagtagatagtagatgttctttttaaactaaaatgctaactgttccgaattcctcatcgcagctaatccgtacatcaaaagacaaaatgctaggtatgtgtactacatctcctgttgctagataagacatatgataggaaacacaccatcaatagtcattgtagctttacttatactacgcatttgcactttcccctgagtggcagaggcgcattgagaaaatcgatctcaacatagtttatgtagcatcccctagatccattactttaagtctccttcgtctttggtgtaggcatgttggacacaacgaggtaaaacacaacacaaacaatgtgtccagcaaagtagtagctgctccagttctccc

SEQ ID NO: 19 - DNA, 7947 bp, synthetic construct
  source 1..7947: mol_type = other DNA; note = Flagellin + Gene 4. Vibrio parahaemolyticus pirBVP)
gggggatccactagttctagagcggccggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacgtaccgggccccccctcgaggtcgacggtatcgataagcttgcgctttttccgagaactccccataagtcaacggctccaatcaagaatgtatccgacaacggcgagcatagcaacacgtccgtctttggagtagaatcatcatgttgtggatgaatacacagatgaatgacattaaaagcatgaacatgttagagagtaggaggtagagattgatatggtagcattgcgatgtttgtttttggtcagcatatgatgagtggataccaatatgatgaaagttgaatctcgcgtttgagctcagcggtacgttattgatcgaaagtagcctgatcaaaatccttggagagtacaagaggatcaaagaatccagtgggggcgataactccaagctcgttctcaaagaggcaatggaggtagaaactcatcccagttgagaagaagtgaaggcagtggcggtggcgaaagcagaggcaacgaggacagacttcctgtgggttgatgcaacgaatatttccagaaggagaagtttagagagttgaaccgctacctacaatgacaaagtatcgtatcgattttgatgttggttggttatgaattcaaactgtaagttggattgtgagaagatcagaagttgaacgaacacatctttccgatcattcacctccacactgcaacaacacggtacttcttccgcggcaggtctctgtcgccattctcttgtcctgttgttggctgtgagacgaggaaagcaacgacaagtttcacaaaagggagttcctttaacgagatatgttttttataaagagtcccaatagaaagacaaattgattcctccgtgcaaacgcgcaaataaacaccacgtccattatatccatatctttcagagtatccaacaagtgttgaaggacaggtagttgaagtaacgtatcttccccctcgactggatccatcaacaaggcgaacaaatccattcaacctctcataaattatctgatttaccaaaccgatatggcacaagtcatcaacaccaactctctctctctcttgacccagaacaacctcaacaagtcccagtccgctctcggaaccgctatcgagcgtctctcttccggactccgtatcaactctgctaaggacgatgctgcaggacaggctatcgctaaccgtttcaccgctaacatcaagggactcactcaggcttcccgtaacgctaacgacggaatctccatcgctcagaccactgagggagctctcaacgagatcaacaacaacctccagcgtgtgcgtgagctcgctgttcagtctgctaactctaccaactcccagtctgacctcgactccatccaggctgagatcacccagcgtctcaacgagatcgaccgtgtgtccggacagactcagttcaacggagtgaaggtcctcgctcaggacaacaccctcaccatccaggttggagccaacgacggagagactatcgatatcgatctcaagcagatcaactctcagaccctcggactcgataccctcaacgtgcaacaaaagtacaaggtctctgataccgctgcaactgttaccggatacgccgatactaccatcgctctcgacaacagtactttcaaggcctctgctactggactcggaggaactgaccagaagatcgatggagatctcaagttcgatgataccactggaaagtactacgccaaggttaccgttaccggaggaactggaaaggatggatactacgaggtttccgttgataagaccaacggaaaggtgactctcgctggaggagctacttccccactcaccggaggactccctgctaccgcaactgaggatgtgaagaacgtgcaagttgcaaacgctgatttgaccgaggctaaggccgcattgaccgcagcaggagttaccggaaccgcatctgttgttaagatgtcttacactgataacaacggaaagactatcgatggaggactcgcagttaaggtgggagatgattactactctgcaactcaaaacaaggatggatccatcagtatcaacactaccaagtacactgcagatgacggaacctccaagactgcactcaacaagctcggaggagcagacggaaagaccgaggttgtttctatcggaggaaagacttacgctgcaagtaaggccgagggacacaacttcaaggcacagcctgatctcgctgaggctgctgctaccaccaccgagaacccactccagaagatcgatgctgctttggcacaggttgacaccctccgttctgacctcggagctgtgcagaaccgtttcaactccgctatcaccaacctcggaaacaccgtgaacaacctcacttctgcccgttctcgtatcgaggattccgactacgctaccgaggtttccaacatgtctcgtgctcagatcctccagcaggccggaacctccgttctcgctcaggctaaccaggttccacaaaacgtcctctctctcctccgtatgactaacgagtacgttgtgactatgtcttctctcactgagttcaacccaaacaacgctcgtaagtcttacctcttcgataactacgaggttgatcctaactacgccttcaaggccatggtttctttcggattgtctaacatcccatacgccggaggattcctctctactttgtggaacatcttctggccaaacactccaaacgagcctgatatcgagaacatctgggagcaattgcgtgatcgtatccaagatctcgttgatgagtctatcatcgatgccatcaacggaatcctcgattctaagatcaaggagactcgtgataagatccaagatatcaacgagaccatcgagaacttcggatacgctgccgctaaggatgattacatcggactcgtgactcactacttgatcggattggaggagaacttcaagcgtgagctcgatggagatgagtggttgggatacgctatcttgcctctcttggccaccactgtctctttgcaaatcacctacatggcttgtggattggattacaaggatgagttcggattcactgattctgatgtccataagctcacccgtaacatcgataagttgtacgatgatgtttcttcttacatcactgagttggccgcttgggccgataacgattcttacaacaacgccaaccaagataacgtttacgatgaggttatgggagcccgttcttggtgtaccgtgcatggattcgagcatatgttgatctggcaaaagatcaaggagttgaagaaggtggatgtgttcgtgcactctaacctcatctcttactctccagctgtgggattcccatctggaaacttcaactacatcgctactggaaccgaggatgagatccctcaaccactcaagccaaacatgttcggagagcgtcgtaaccgtatcgtgaagatcgagtcttggaactctatcgagatccactactacaaccgtgttggacgtttgaagctcacctacgagaacggagaggtggtcgagttgggaaaggcccataagtacgatgagcattaccaatctatcgagctcaacggagcctacatcaagtacgttgatgtgatcgccaacggacctgaggctatcgatcgtatcgtcttccacttctctgatgatcgtaccttcgtggttggagagaactctggaaagccatctgtgcgtctccaactcgagggacacttcatctgtggaatgttggctgatcaagagggatctgataaggttgccgctttctctgtcgcttacgagctcttccacccagatgagttcggaactgagaagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcatactggattggtgaatcaatgagccgtagcacaatggttacattcggctagctaagatccaatggcaaggaccaagtgctggaacttgttttgctttagcagatcttagcgtgagaggtatttgtcctctgtcaggagtagatagtagatgttctttttaaactaaaatgctaactgttccgaattcctcatcgcagctaatccgtacatcaaaagacaaaatgctaggtatgtgtactacatctcctgttgctagataagacatatgataggaaacacaccatcaatagtcattgtagctttacttatactacgcatttgcactttcccctgagtggcagaggcgcattgagaaaatcgatctcaacatagtttatgtagcatcccctagatccattactttaagtctccttcgtctttggtgtaggcatgttggacacaacgaggtaaaacacaacacaaacaatgtgtccagcaaagtagtagctgctccagttctccc